(19) United States
(12) Patent Application Publication
(10) Pub. No.: US 2024/0316200 A1
(43) Pub. Date: Sep. 26, 2024

(54) BICISTRONIC CONSTRUCTS FOR ALLOGENEIC GENE THERAPY

(71) Applicant: Lung Biotechnology PBC, Silver Spring, MD (US)

(72) Inventors: Colin EXLINE, Silver Spring, MD (US); Jintang DU, Silver Spring, MD (US); Sean STEVENS, Silver Spring, MD (US)

(21) Appl. No.: 18/611,477

(22) Filed: Mar. 20, 2024

Related U.S. Application Data
(60) Provisional application No. 63/491,492, filed on Mar. 21, 2023.

(51) Int. Cl. A61K 39/00; A61K 48/00; A61P 35/00; C12N 9/22; C12N 15/11; C12N 15/90

(52) U.S. Cl. CPC A61K 39/464471; A61K 39/4611; A61K 39/4613; A61K 39/4614; A61K 39/4615; A61K 39/4631; A61K 48/0033; A61P 35/00; C12N 9/22; C12N 15/11; C12N 15/907; A61K 2039/575; C12N 2840/203

(57) ABSTRACT

The present disclosure relates to bicistronic polypeptide constructs for use in allogeneic gene therapy, for example, CAR-T cell therapy. The bicistronic constructs comprise first polynucleotide encoding a therapeutic molecule (e.g., a CAR-T or an antibody) and a second polynucleotide encoding an Immune Surveillance Masking Molecule (ISMM). The ISMM comprises a human leukocyte antigen-E genetically fused to a non-functional version, e.g., a fragment, of the protein knock-out by the insertion of the bicistronic construct, e.g., beta-2 microglobulin or B2M. Also provided are vectors comprising the bicistronic constructs, cells (e.g., CAR-T cells), and methods of use. Also provided are kits and articles of manufacture. The present disclosure also provides four novel insertion sites that can be used to insert an expression construct in the B2M gene.

### CROSS-REFERENCE TO RELATED APPLICATIONS AND INCORPORATION BY REFERENCE

This application claims the priority benefit of U.S. Provisional Application No. 63/491,492, filed on Mar. 21, 2023, which is herein incorporated by reference in its entirety.

### REFERENCE TO SEQUENCE LISTING SUBMITTED ELECTRONICALLY

The content of the electronically submitted ST.26 sequence listing in XML format (Name 5064_0030002_SequenceListing_ST26.xml; Size: 158,640 bytes; and Date of Creation: Mar. 18, 2023) filed with the application is incorporated herein by reference in its entirety.

### FIELD

The present disclosure provides bicistronic constructs for allogeneic gene therapy.

### BACKGROUND

Gene therapy, in particular, adaptive cellular immunotherapy using immune cells expressing chimeric antigenic receptors (CARs) has shown promise, particularly for the treatment of malignancies. Adoptive cellular therapy refers to the isolation of immune cells, followed by ex vivo manipulation and subsequent delivery into patients as a therapeutic intervention. CARs combine the specificity of an antibody with signaling domains of effector cells and costimulatory molecules. When constitutively expressed on the surface of an immune cell through non-viral or viral transduction, CARs enable an effector cell to recognize targets in an antigen-specific manner. CARs designed to target a specific tumor-associated-antigen (TAA) can then be used for anticancer therapy.

The majority of clinically evaluated CAR products are derived from autologous immune cells, i.e., cells harvested from the patient that will undergo therapy. The autologous (patient-derived) CAR T-cell paradigm has several important advantages, including infusion of CAR-engineered cell products without immunologic mismatch between donor and recipient. This strategy, however, has significant clinical and economic constraints, for example, the availability of a facility to perform successful leukapheresis and obtain the cells from a patient (e.g., a patient with relapsed/refractory malignant disease), shipment of the cells to and from processing centers, quality of the cells (which may have been negatively affected by previous aggressive cancer-directed therapies), and time required to manufacture and test the cells prior to clinical use. Time delays can be significant, particularly in patients with aggressive relapsed/resistant cancers, who are at risk of clinical deterioration, which could preclude proceeding with CAR cell therapy. In addition, generation of a cell product is not guaranteed and for those patients for whom a product can be successfully generated, a proportion of products have limited short- or long-term efficacy. This is likely in part due to poor autologous immune cell fitness in cancer patients, particularly following aggressive cancer-directed therapies. Lastly, autologous cell therapy is performed for individual patients and is associated with significant costs, limiting broader applications of this therapy.

Using allogeneic cells, i.e., cells obtained from a healthy donor who is not the subject who will ultimately be receiving the gene therapy, has the potential to overcome many of these limitations. For example, the use of allogeneic cells allows the generation of CAR-engineered cell products to be cost effective, readily available, and provide a higher quality product. Healthy donor cells confer a uniform starting material, allowing for more predictable manufacturing and performance of the generated cell product. Allogeneic therapies have the potential to provide an “off the shelf” immunotherapeutic solution, such that a single manufacturing run would allow dosing for several patients and/or multiple dosing for individual patients. In addition, by increasing production scale and creating a bank of manufactured CAR immune cells from healthy donors, the cost-per-patient would decrease while access to product would increase. However, allogeneic CAR cell products can potentially induce graft-versus-host-disease or risk immune-mediated rejection by the host, limiting the therapeutic effect.

### BRIEF SUMMARY

The present disclosure provides a bicistronic polynucleotide encoding a (i) therapeutic agent and (ii) an immune surveillance masking molecule (ISMM), wherein the ISMM comprises a beta-2-microglobulin (B2M) non-functional polypeptide and a human leukocyte antigen (HLA). In some aspects, the therapeutic agent is a chimeric antigen receptor (CAR) comprising an antigen-binding domain that specifically binds to an epitope on a tumor antigen on a target cell. In some aspects, the antigen-binding domain comprises an antibody or an antigen-binding portion thereof. In some aspects, the tumor antigen is disialoganglioside GD2. In some aspects, the antibody is dinutuximab or an antigen-binding portion thereof. See www.accessdata.fda.gov/drugsatfda_docs/label/2015/125516s000lbl.pdf, which is herein incorporated by reference in its entirety.

In some aspects, the antibody is a single-chain variable fragment (scFv) comprising the variable region of the heavy chain (VH) and the variable region of the light chain (VL) of dinutuximab. In some aspects, the dinutuximab scFv comprises the protein sequence set forth in SEQ ID NO: 22. In some aspects, the antigen-binding domain cross-competes with dinutuximab. In some aspects, the antigen-binding domain binds to the same epitope as dinutuximab. In some aspects, the antigen-binding domain comprises VH CDR3 of dinutuximab. In some aspects, the antigen-binding domain further comprises a VH CDR1 and a VH CDR2. In some aspects, the VH CDR1 comprises the VH CDR1 of dinutuximab and/or the VH CDR2 comprises the VH CDR2 of dinutuximab. In some aspects, the antigen-binding domain further comprises a VL CDR1, VL CDR2, and/or VL CDR3. In some aspects, VL CDR1 comprises the VL CDR1 of dinutuximab, the VL CDR2 comprises the VL CDR2 of dinutuximab, and/or the VL CDR3 comprises the VL CDR3 of dinutuximab. In some aspects, the antigen-binding domain comprises (i) VH CDR1 of SEQ ID NO: 59; VH CDR2 of SEQ ID NO: 63; and VH CDR3 of SEQ ID NO: 67; and/or VL CDR1 of SEQ ID NO: 71; VL CDR2 of SEQ ID NO: 75; and VL CDR3 of SEQ ID NO: 79; or, (ii) VH CDR1 of SEQ ID NO: 60; VH CDR2 of SEQ ID NO: 64; and VH CDR3 of SEQ ID NO: 68; and/or VL CDR1 of SEQ ID NO: 72; VL CDR2 of SEQ ID NO: 76; and VL CDR3 of SEQ ID NO: 80; or, (iii) VH CDR1 of SEQ ID NO: 61; VH CDR2 of SEQ ID NO: 65; and VH CDR3 of SEQ ID NO: 69; and/or VL CDR1 of SEQ ID NO: 73; VL CDR2 of SEQ ID NO: 77; and VL CDR3 of SEQ ID NO: 81; or, (iv) VH CDR1 of SEQ ID NO: 62; VH CDR2 of SEQ ID NO: 66; and VH CDR3 of SEQ ID NO: 70; and/or VL CDR1 of SEQ ID NO: 74; VL CDR2 of SEQ ID NO: 78; and VL CDR3 of SEQ ID NO: 82; or, (v) VH CDR1 of SEQ ID NO: 53; VH CDR2 of SEQ ID NO: 54; and VH CDR3 of SEQ ID NO: 55; and/or VL CDR1 of SEQ ID NO: 56; VL CDR2 of SEQ ID NO: 57; and VL CDR3 of SEQ ID NO: 58.

In some aspects, the antigen-binding domain comprises a VH and a VL, and wherein the VH comprises the protein sequence set forth in SEQ ID NO: 44 or the VL comprises the protein sequence set forth in SEQ ID NO: 46. In some aspects, the antigen-binding domain comprises a VH comprising the protein sequence set forth in SEQ ID NO:44 and a VL comprising the protein sequence set forth in SEQ ID NO: 46. In some aspects, the VH and VL are connected via a linker. In some aspects, the VH and VL are connected in a VH-linker-VL or VL-linker-VH conformation. In some aspects, the linker is a Gly₄-Ser linker. In some aspects, the Gly₄-Ser linker comprises the sequence set forth in SEQ ID NO: 84. In some aspects, the CAR construct is designed as a standard CAR, a split CAR, an off-switch CAR, an on-switch CAR, a first-generation CAR, a second-generation CAR, a third-generation CAR, or a fourth-generation CAR.

In some aspects, the antigen-binding domain is an lg NAR, a Fab, a Fab′, a F(ab)′2, a F(ab)′3, an Fv, a single chain variable fragment (scFv), a bis-scFv, a (scFv)2, a minibody, a diabody, a triabody, a tetrabody, an intrabody, a disulfide stabilized Fv protein (dsFv), a unibody, a nanobody, an affibody, a DARPin, a monobody, an adnectin, an alphabody, or a designed binder. In some aspects, the CAR construct further comprises a transmembrane domain, an intracellular domain, and a spacer located between the antigen-binding domain and the transmembrane domain. In some aspects, the intracellular domain of the CAR construct is a signaling domain derived from CD3zeta, FcR gamma, FcR beta, CD3 gamma, CD3 delta, CD3 epsilon, CD5, CD22, CD79a, CD79b, CD66d, or CD28. In some aspects, the intracellular domain of the CAR construct is derived from CD28. In some aspects, the transmembrane domain of the CAR construct is derived from CD28. In some aspects, the transmembrane domain is linked to the intracellular domain by a linker. In some aspects, the intracellular domain and transmembrane domain of the CAR construct are derived from the same molecule. In some aspects, the transmembrane and intracellular domain are derived from CD28. In some aspects, the spacer of the CAR construct is a CD8alpha hinge. In some aspects, the CAR construct further comprises a co-stimulatory domain or a combination thereof. In some aspects, the co-stimulatory domain is derived from 2B4, HVEM, ICOS, LAG3, DAP10, DAP12, CD27, CD28, 4-1BB (CD137), OX40 (CD134), CD30, CD40, ICOS (CD278), glucocorticoid-induced tumor necrosis factor receptor (GITR), lymphocyte function-associated antigen-1 (LFA-1), CD2, CD7, LIGHT, NKG2C, B7-H3, CD3zeta, and combinations thereof. In some aspects, the co-stimulatory domain comprises a 4-1BB activation domain. In some aspects, the co-stimulatory domain comprises a CD3zeta activation domain. In some aspects, the co-stimulatory domain comprises a 4-1BB activation domain and a CD3zeta activation domain. In some aspects, the CAR construct comprises the nucleic acid sequence set forth in SEQ ID NO: 19. In some aspects, the CAR construct encodes the protein set forth in SEQ ID NO: 20.

In some aspects, the therapeutic agent comprises an antibody or antigen binding portion thereof, an enzyme, a receptor, a cytokine, a clotting factor, or a hormone. In some aspects, the B2M non-functional polypeptide is a B2M non-functional fragment. In some aspects, the B2M non-functional polypeptide is a B2M non-functional variant. In some aspects, the HLA is HLA-E or HLA-G. In some aspects, the B2M polypeptide and the HLA are connected by a linker. In some aspects, the linker is a Gly4-Ser linker. In some aspects, the Gly4-Ser linker comprises the sequence set forth in SEQ ID NO: 84. In some aspects of the bicistronic polynucleotide of the present disclosure, (i) the nucleic acid sequence encoding the therapeutic agent and the nucleic acid sequence encoding the ISMM are connect by a 2A (e.g., P2A) element; or, (ii) the nucleic acid sequence encoding the therapeutic agent and the nucleic acid sequence encoding the ISMM are connect by an Internal Ribosome Entry Site (IRES).

In some aspects, the nucleic acid sequence encoding the therapeutic agent comprises the sequence set forth in SEQ ID NO: 5. In some aspects, the nucleic acid sequence encoding the ISMM comprises the sequence set forth in SEQ ID NO: 6. In some aspects, the nucleic acid sequence encoding the therapeutic agent comprises the sequence set forth in SEQ ID NO: 5, the nucleic acid sequence encoding the ISMM comprises the sequence set forth in SEQ ID NO: 6. In some aspects, the bicistronic polynucleotide is selected from the group consisting of Bicistronic Construct 1, Bicistronic Construct 2, Bicistronic Construct 3, Bicistronic Construct 4, Bicistronic Construct 5, Bicistronic Construct 6, Bicistronic Construct 7 or Bicistronic Construct 8. In some aspects, the bicistronic polynucleotide further comprises a 5′ sequence complementary to a B2M gene sequence upstream from an insertion site and a 3′ sequence complementary to a B2M gene sequence downstream from the insertion site. In some aspects, the 5′ sequence and 3′ sequence have the same length. In some aspects, the 5′ sequence and 3′ sequence are at least about 100 nucleotides, at least about 200 nucleotides, at least about 300 nucleotides, at least about 400 nucleotides, at least about 500 nucleotides, at least about 600 nucleotides, at least about 700 nucleotides, at least about 800 nucleotides, at least about 900 nucleotides, at least about 1000 nucleotides in length. In some aspects, the bicistronic polynucleotide is selected from the group consisting of Full Donor 1, Full Donor 2, Full Donor 3, Full Donor 4, Full Donor 5, Full Donor 6, Full Donor 7, or Full Donor 8.

In some aspects, the bicistronic polynucleotide is inserted in the B2M gene, and insertion in the B2M gene inactivates the gene. In some aspects, the insertion in the B2M gene is mediated by a nuclease. In some aspects, insertion in the B2M gene is mediated by a CRISPR/Cas nuclease. In some aspects, the nuclease is CRISPR/Cas9.

In some aspects, the insertion site in the B2M gene is at an intron location. In some aspects, the insertion site in the B2M gene is at an intron-exon junction location. In some aspects, the insertion site in the B2M gene is at an exon location. In some aspects, the exon location is at exon 1. In some aspects, the insertion site is Site 1 (ACTCTCTCTTTCTGGCCTGG; SEQ ID NO: 1). In some aspects, the exon location is at exon 2. In some aspects, the insertion site is Site 2 (AGTCACATGGTTCACACGGC; SEQ ID NO:2) or Site 3 (CACAGCCCAAGATAGTTAAG; SEQ ID NO:3). In some aspects, the exon location is at exon 3. In some aspects, the insertion site is Site 4 (GAGACATGTAAGCAGCATCA; SEQ ID NO: 4). In some aspects, the polynucleotide is a DNA molecule, or a RNA molecule. In some aspects, the CAR is an inducible CAR.

The present disclosure also provides a vector comprising a bicistronic polynucleotide disclosed herein which is operably linked to a regulatory element. In some aspects, the vector is a viral vector, a mammalian vector, or bacterial vector. In some aspects, the vector is a retroviral vector. In some aspects, the viral vector is selected from the group consisting of an adenoviral vector, a lentivirus, a Sendai virus vector, a baculoviral vector, an Epstein Barr viral vector, a papovaviral vector, a vaccinia viral vector, a herpes simplex viral vector, a hybrid vector, and an adeno associated virus (AAV) vector.

The present disclosure also provides a composition comprising a bicistronic polynucleotide disclosed herein or a vector comprising a bicistronic polynucleotide disclosed herein. Also provided is a kit comprising (i) a bicistronic polynucleotide disclosed herein, (ii) a vector comprising a bicistronic polynucleotide disclosed herein, or (iii) a composition comprising a bicistronic polynucleotide disclosed herein or a vector comprising a bicistronic polynucleotide disclosed herein.

Also provided is a cell genetically modified to express a therapeutic agent and an ISMM, comprising (i) a bicistronic polynucleotide disclosed herein, (ii) a vector comprising a bicistronic polynucleotide disclosed herein, or (iii) a composition comprising a bicistronic polynucleotide disclosed herein or a vector comprising a bicistronic polynucleotide disclosed herein. In some aspects, the cell is a T cell, a natural killer (NK) cell, a natural killer T (NKT) cell, an ILC cell, a macrophage, or an antigen-presenting cell. In some aspects, the cell is allogeneic. The some aspects, the genetically modified cell is part of kit or article of manufacture.

The present disclosure also provides a composition comprising (i) a bicistronic polynucleotide disclosed herein, (ii) a vector comprising a bicistronic polynucleotide disclosed herein, (iii) a composition comprising a bicistronic polynucleotide disclosed herein or a vector comprising a bicistronic polynucleotide disclosed herein, or a cell comprising (i), (ii), or (iii). In some aspects, such composition is used for treating a subject in need of therapy. In some aspects, the therapy is a CAR therapy. In some aspects, the composition is part of a kit or article of manufacture.

The present disclosure also provides a pharmaceutical composition for treating cancer in a subject in need thereof wherein the pharmaceutical composition comprises a cell genetically modified to express a therapeutic agent and an ISMM, comprising (i) a bicistronic polynucleotide disclosed herein, (ii) a vector comprising a bicistronic polynucleotide disclosed herein, or (iii) a composition comprising a bicistronic polynucleotide disclosed herein or a vector comprising a bicistronic polynucleotide disclosed herein. In some aspects, the pharmaceutical composition is part of a kit or article of manufacture.

The present disclosure also provides a pharmaceutical composition for treating cancer in a subject in need thereof wherein the pharmaceutical composition comprises
(i) a bicistronic polynucleotide disclosed herein;
(ii) a vector comprising a bicistronic polynucleotide disclosed herein;
(iii) a composition comprising a bicistronic polynucleotide disclosed herein or a vector comprising a bicistronic polynucleotide disclosed herein; or,
(iv) a cell comprising any of (i), (ii) or (iii).

Also provided is the use of
(i) a bicistronic polynucleotide disclosed herein;
(ii) a vector comprising the bicistronic polynucleotide of (i);
(iii) a composition comprising (i) or (ii);
(iv) a kit comprising (i), (ii) or (iii);
(v) a cell comprising any of (i), (ii), or (iii);
(vi) a composition comprising (v);
(vii) a pharmaceutical composition comprising (i), (ii), (iii), (v), or (vi); or,
(viii) a kit comprising (v), (vi) or (vii)
as a medicament.

Also provided is the use of
(i) a bicistronic polynucleotide disclosed herein;
(ii) a vector comprising the bicistronic polynucleotide of (i);
(iii) a composition comprising (i) or (ii);
(iv) a kit comprising (i), (ii) or (iii);
(v) a cell comprising any of (i), (ii), or (iii);
(vi) a composition comprising (v);
(vii) a pharmaceutical composition comprising (i), (ii), (iii), (v), or (vi); or,
(viii) a kit comprising (v), (vi) or (vii)
as a medicament for treating cancer or an inflammatory disease or condition in a subject in need thereof.

Also provided is the use of
(i) a bicistronic polynucleotide disclosed herein;
(ii) a vector comprising the bicistronic polynucleotide of (i);
(iii) a composition comprising (i) or (ii);
(iv) a kit comprising (i), (ii) or (iii);
(v) a cell comprising any of (i), (ii), or (iii);
(vi) a composition comprising (v);
(vii) a pharmaceutical composition comprising (i), (ii), (iii), (v), or (vi); or,
(viii) a kit comprising (v), (vi) or (vii)
for the manufacture of a medicament for treating cancer or an inflammatory disease or condition in a subject in need thereof.

The present disclosure also provides a method of stimulating a T cell-mediated immune response to a target cell population or tissue in a subject, comprising administering an effective amount of the cell comprising a bicistronic polynucleotide disclosed herein to the subject. Also provided is a method of providing an anti-tumor immunity in a subject in need thereof, the method comprising administering to the subject an effective amount of a cell comprising a bicistronic polynucleotide disclosed herein. The present disclosure provides a method of treating cancer in a subject in need thereof comprising administering to the subject an effective amount of a cell comprising a bicistronic polynucleotide disclosed herein.

The present disclosure provides a method of preparing a population of cells for a therapy comprising transducing a population of cells isolated from a subject with a bicistronic polynucleotide, vector, or composition disclosed herein. In some aspects, the transduction comprises culturing the cell under suitable condition. In some aspects, the therapy is allogeneic cell therapy.

The present disclosure provides a method of generating a persisting population of genetically engineered cells in a subject diagnosed with cancer or an inflammatory disease, the method comprising administering to the subject a cell genetically engineered to express a bicistronic polynucleotide disclosed herein. Also provided in a method of expanding a population of genetically engineered cells in a subject diagnosed with cancer or an inflammatory disease, the method comprising administering to the subject a cell genetically engineered to express a bicistronic polynucleotide disclosed herein. In some aspects, the cell is a T cell. In some aspects, the T cell is an allogeneic T cell. In some aspects, the subject is a human subject.

The present disclosure also provides a method to generate an allogeneic cell for gene therapy comprising inserting a bicistronic construct comprising a nucleic acid encoding a therapeutic agent and a nucleic acid encoding an ISMM in the B2M gene, wherein insertion of the bicistronic construct inactivates the B2M gene. In some aspects, the nucleic acid encoding the ISMM comprises a nucleic acid encoding a HLA selected from HLA-E or HLA-G, or a functional variant thereof. In some aspects, the gene therapy is CAR-T therapy. In some aspects, the insertion site in the B2M gene is selected from Site 1 (ACTCTCTCTTTCTGGCCTGG; SEQ ID NO: 1); Site 2 (AGTCACATGGTTCACACGGC; SEQ ID NO:2); Site 3 (CACAGCCCAAGATAGTTAAG; SEQ ID NO:3); or Site 4 (GAGACATGTAAGCAGCATCA; SEQ ID NO: 4). In some aspects, the insertion site comprises a sequence located about 10 nucleotides, about 20 nucleotides, about 30 nucleotides, about 40 nucleotides, about 50 nucleotides, about 60 nucleotides, about 70 nucleotides, about 80 nucleotides, about 90 nucleotides, or about 100 nucleotides upstream or downstream with respect to the 5′ end or 3′ end of Site 1, Site 2, Site 3 or Site 3. In some aspects, the insertion site is an insertion site that overlaps with Site 1, Site 2, Site 3, or Site 4. In some aspect, the insertion site is in a corresponding location on the antiparallel strand.

The present disclosures provides an allogeneic CAR-T cell comprising specific a bicistronic construct selected from bicistronic constructs BC1, BC2, BC3, BC4, BC5, BC6, BC7 or BC8, wherein the nucleic acid sequence encoding HLA-E has been replaced with a nucleic acid sequence encoding HLA-G. In some aspects, the present disclosure provides an allogeneic CAR-T cell comprising a specific bicistronic construct selected from bicistronic construct BC1, BC2, BC3, BC4, BC5, BC6, BC7 or BC8, wherein the nucleic acid sequence encoding a fragment of B2M has been replaced with a nucleic acid sequence encoding a fragment of TRAC, and wherein the bicistronic construct is inserted in the TRAC gene. In some aspects, the present disclosure provides an allogeneic CAR-T cell comprising a specific bicistronic construct selected from bicistronic construct BC1, BC2, BC3, BC4, BC5, BC6, BC7 or BC8, wherein the nucleic acid sequence encoding a fragment of B2M has been replaced with a nucleic acid sequence encoding a fragment of CD52, and wherein the bicistronic construct is inserted in the CD52 gene.

The present disclosure provides a kit comprising a gRNA for CRISPR/Cas9 mediated insertion in the B2M wherein the gRNA is selected from Site 1 gRNA (ACTCTCTCTTTCTGGCCTGG; SEQ ID NO: 1); Site 2 gRNA (AGTCACATGGTTCACACGGC; SEQ ID NO:2); Site 3 gRNA (CACAGCCCAAGATAGTTAAG; SEQ ID (GAGACATGTAAGCAGCATCA; SEQ ID NO: 4). NO:3); and Site 4 gRNA

### BRIEF DESCRIPTION OF THE DRAWINGS/FIGURES

FIG. 1 shows the sequences of the four B2M insertion sites disclosed herein, and a schematic representation indicating the location of each insertion site on the B2M gene.

FIGS. 2A-2E show density plots of flow cytometry assays performed to measure the cell surface expression levels of B2M and HLA-A/B/C. FIG. 2A: Control cells (Mock) in which the endogenous B2M locus remained intact. FIG. 2B: Disruption of the endogenous B2M locus with CRISPR reagent targeting Site 1 (SEQ ID NO: 1). FIG. 2C: Disruption of the endogenous B2M locus with CRISPR reagent targeting Site 2 (SEQ ID NO: 2). FIG. 2D: Disruption of the endogenous B2M locus with CRISPR reagent targeting Site 3 (SEQ ID NO: 3). FIG. 2E: Disruption of the endogenous B2M locus with CRISPR reagent targeting Site 4 (SEQ ID NO: 4).

FIGS. 3A and 3B show schematic representations of the CAR and HLA-E elements used to design bicistronic constructs. FIG. 3A is a schematic representation of the anti-GD2scFv+CD8aH+CD28TM/IC+4-1BBAD+CD3zetaAD chimeric antigen receptor (anti-GD2 CAR, SEQ ID NO: 5) component of the bicistronic constructs. From N-terminus to C-terminus, the CAR component (labeled “CAR”) comprises the following operably linked elements following a signal peptide: (1) an anti-GD2 scFv derived from dinutuximab; (2) a CD8alpha hinge; (3) a CD28 transmembrane domain (TM) and CD28 intracellular domain, which are jointly labeled as CD28; (4) a 4-1BB activation domain; and, (5) a CD3zeta activation domain. FIG. 3A is a schematic representation of the B2M+HLA-E immune surveillance masking molecule (ISMM) (SEQ ID NO: 6) component of the bicistronic constructs. The ISMM component (labeled “HLA-E”) comprises from N-terminus to C-terminus a non-functional fragment of the beta-2 microglobulin gene (B2M), a 4-repeat Gly₄-Ser linker (i.e., (Gly+Ser)₄), and mature human leukocyte antigen-E (HLA-E).

FIG. 3C is a schematic representation showing generally the strategy used to insert bicistronic constructs comprising ISMM and CAR elements separated by either P2A element or an IRES into specific locations of the B2M gene of donor cells using CRISPR/Cas and homologous recombination. The result would be a donor cell (allogeneic cell) that could be used for CAR T-cell therapy having an inactivated B2M gene, which would reduce allogeneic response, but would have HLA-E expression that would protect the cell against allogeneic natural killer (NK) cell-mediated lysis.

FIGS. 4A and 4B show schematic representations of bicistronic constructs for insertion in B2M Site 1. FIG. 4A corresponds to Bicistronic Construct 1 (BC1) which has a 2A (P2A) element between the ISMM and CAR components of the construct. FIG. 4B corresponds to Bicistronic Construct 2 (BC2) which has an IRES element between the ISMM and CAR components of the construct.

FIGS. 5A and 5B show schematic representations of bicistronic constructs for insertion in B2M Sites 2 or 3. FIG. 5A corresponds to Bicistronic Construct 3 (BC3) when inserted in Site 2 or Bicistronic Construct 5 (BC5) when inserted in Site 3, and has a 2A (P2A) element between the ISMM and CAR components of the construct. FIG. 5B corresponds to Bicistronic Construct 4 (BC4) when inserted in Site 2 or Bicistronic Construct 5 (BC6) when inserted in Site 3, and has an IRES element between the ISMM and CAR components of the construct.

FIGS. 6A and 6B show schematic representations of bicistronic constructs for insertion in B2M Site 4. FIG. 6A corresponds to Bicistronic Construct 7 (BC7) which has a 2A (P2A) element between the ISMM and CAR components of the construct. FIG. 6B corresponds to Bicistronic Construct 8 (BC8) which has an IRES element between the ISMM and CAR components of the construct.

FIGS. 7A and 7B show schematic representations of the full donors generated from BC1 and BC2, named respectively, Full Donor 1 (FD1) and Full Donor 2 (FD2) by adding 1,000 bp of the B2M gene flanking CRISPR Site 1 (500 bp on each side) to create homology arms to drive site specific recombination in frame.

FIGS. 8A and 8B show schematic representations of the full donors generated from BC3, BC4, BC5 and BC6, named respectively, Full Donor 3 (FD3), Full Donor 4 (FD4), Full Donor 5 (FD5) and Full Donor 6 (FD6) by adding 1,000 bp of the B2M gene flanking CRISPR Site 2 or Site 3 (500 bp on each side) to create homology arms to drive site specific recombination in frame.

FIGS. 9A and 9B show schematic representations of the full donors generated from BC7 and BC8, named respectively, Full Donor 7 (FD7) and Full Donor 8 (FD8) by adding 1,000 bp of the B2M gene flanking CRISPR Site 4 (500 bp on each side) to create homology arms to drive site specific recombination in frame.

FIGS. 10A-10D show density plots of flow cytometry assays performed to measure cell surface expression levels of HLA-A/B/C and HLA-E. FIG. 10A: Untreated control cells (Mock). FIG. 10B: Insertion of FD2 in Site 1 of the B2M gene. FIG. 10C: Insertion of FD6 in Site 3 of the B2M gene. FIG. 10D: Insertion of FD8 in Site 4 of the B2M gene.

FIGS. 11A-11D show density plots of flow cytometry assays performed to measure cell surface expression levels of HLA-A/B/C and HLA-E. FIG. 11A: Untreated control cells (Mock). FIG. 11B: Insertion of FD1 in Site 1 of the B2M gene. FIG. 11C: Insertion of FD5 in Site 3 of the B2M gene. FIG. 11D: Insertion of FD6 in Site 3 of the B2M gene.

FIG. 12 shows a plot representing the percentage change (%) of CHP-134 neuroblastoma cells present in co-culture with transformed T-cell at different ratios. (1:1, 2:1, 4:1, or 10:1), up to 24 hours following the beginning of co-culture. All the transformed T-cells used in the experiment contained bicistronic constructs targeted to B2M insertion Site 1 having the CAR portion and ISMM portion of the construct connected by a 2A element (P2A), i.e., Bicistronic Construct 1 (see FIG. 4A).

FIG. 13 shows a plot representing the percentage change (%) of CHP-134 neuroblastoma cells present in co-culture with transformed T-cell at different ratios. (1:1, 2:1, 4:1, or 10:1), up to 24 hours following the beginning of co-culture. All the transformed T-cells used in the experiment contained bicistronic constructs targeted to B2M insertion Site 3 having the CAR portion and ISMM portion of the construct connected by a 2A element (P2A), Bicistronic Construct 5 (see FIG. 5A).

FIG. 14 shows the plot representing the percentage change (%) of CHP-134 neuroblastoma present in co-culture with transformed T-cell at different ratios. (1:1, 2:1, 4:1, or 10:1) up to 24 hours following the beginning of co-culture. All the transformed T-cells used in the experiment contained bicistronic constructs targeted to B2M insertion Site 3 having the CAR portion and ISMM portion of the construct connected by IRES, Bicistronic Construct 6 (see FIG. 5B).

### DETAILED DESCRIPTION

The present disclosure provides a bicistronic polynucleotide encoding a (i) therapeutic agent and (ii) an immune surveillance masking molecule (ISMM), wherein the ISMM comprises a non-functional beta-2-microglobulin (B2M) polypeptide and a human leukocyte antigen (HLA). As used herein, the terms “immune surveillance masking molecule” and “ISMM” refer to a polynucleotide construct and its polypeptide product, e.g., a construct encoding a B2M non-functional polypeptide (e.g., a non-functional fragment or non-functional variant thereof), and an HLA polypeptide or a functional fragment or functional variant thereof, wherein the expression of the construct decreases immunogenicity.

Beta-2-microglobulin (abbreviated “B2M”) is a serum protein found in association with the major histocompatibility complex (MHC) class I heavy chain on the surface of nearly all nucleated cells. The inactivation of the B2M gene can prevent alloantigen presentation by infused T-cells. Due to the lack of functional B2M, recipient T-cell recognition of allogeneic CAR T cells via interaction of HLA/MHC is hindered. Nevertheless, the absence of functional B2M expression in engineered T-cells may trigger an immune response, since they may still be recognized as foreign cells. For that reason, the bicistronic constructs of the present disclosure, in addition to inactivating B2M via their insertion in the B2M gene, include a polynucleotide sequence encoding HLA-E (or, alternatively, HLA-G).

HLA class I histocompatibility antigen, alpha chain E, also known as MHC class I antigen E (abbreviated “HLA-E”), is a protein that in humans is encoded by the HLA-E gene. The human HLA-E is a non-classical MHC class I molecule that is characterized by a limited polymorphism and a lower cell surface expression than its classical paralogues, and is common to all humans.

The expression of the construct encoding the partial but inactive B2M fused in frame with the HLA-E molecule allows the immune system to sense that the genetically engineered cells, although not expressing B2M, are human and not a danger. As the CAR-T cell expressing the HLA-E molecule are not seen as foreign despite the lack of expression of functional B2M, they do not trigger an immune response, and particular NK cell fratricide via “missing cell”-induced lysis.

The insertion of the bicistronic polynucleotides of the present disclosure in specific insertion sites in the B2M gene accomplishes two important tasks. First, the insertion of the bicistronic polynucleotide inactivates the B2M gene, therefore generating a cell that can be used for allogeneic therapy, e.g., for CAR-T gene therapy, or gene replacement therapy. For example, inserting in the B2M gene a CAR against a certain type of cancer, or inserting a functional copy of a gene to compensate for the existence of a defective gene (e.g., a blood coagulation factor, an enzyme, or a hormone). Second, the generation of the allogeneic cells for gene therapy is simplified because the inactivation of B2M and the insertion of the therapeutic gene take place in the same operation. Furthermore, the insertion of the bicistronic polynucleotide in the existing B2M gene results in an expression process that is controlled by the endogenous B2M promoter, obviating the need to provide an exogenous promoter. A consequence of the use of the B2M promoter is that regulatory signals that control the expression and homeostasis of B2M are equally able to regulate the expression of the engineered bicistronic constructs of the present disclosure, which are not expressed at levels excessively high or excessively low, as it could be the case if the expression of the bicistronic constructs was under the control of an exogenous promoter.

Remarkably, the use of a 2A element (e.g., P2A) to separate the therapeutic agent (e.g., CAR portion of the construct) and ISMM portions of the bicistronic polynucleotides disclosed herein has been seen to have a large positive impact on expression with respect to the use of IRES.

The cell engineering approach to generate allogeneic cells presented here can be extended beyond B2M. This approach can be used, for example, to insert analogous bicistronic polypeptides in alternative or additional genes that can be knocked-out to generate allogeneic cells, for example, a bicistronic construct of the present disclosure may be inserted in the T Cell Receptor Alpha Constant gene (TRAC), the Programmed cell death protein 1 gene (PDCD1, also known as PD-1 or CD279) gene, the CD52 gene, the SAG gene (S-arrestin), or any combination thereof. Thus, in some aspects, the methods disclosed herein may be used to insert one or more bicistronic polynucleotides at one or more locations in the B2M, TRAC, PDCD1, SAG, or CD52 genes, or a combination thereof. This approach can be used also, for example, to insert analogous bicistronic polypeptides in additional genes that are generally knocked-out to increase the potency of allogeneic cells, for example, CD5. Thus, in some aspects, the methods disclosed herein may be used to insert one or more bicistronic polynucleotides at one or more locations in the (15 gene.

Similarly, the cell engineering approach to generate allogeneic cells expressing a CAR disclosed can be extended to other therapeutic proteins, for example, antibodies, enzymes, clotting factors, etc. Likewise, the cell engineering methods disclosed herein can be applied to generate allogeneic cells comprising polycistronic polynucleotides comprising one or more ISMM (e.g., an ISMM comprising HLA-E and a second ISMM comprising HLA-G) and one or more therapeutic proteins (e.g., the heavy chain and the light chain of an antibody, or multiple subunits of a therapeutic protein).

Before the present disclosure is described in detail, it is to be understood that this disclosure is not limited to the particular compositions or process steps described, and such compositions or process steps can vary. As will be apparent to those of skill in the art upon reading this disclosure, each of the individual aspects described and illustrated herein has discrete components and features which can be readily separated from or combined with the features of any of the other several aspects without departing from the scope or spirit of the present disclosure. Any recited method can be carried out in the order of events recited or in any other order that is logically possible.

Headings provided herein are not limitations of the various aspects of the disclosure, which can be defined by reference to the specification as a whole. It is also to be understood that the terminology used herein is for describing particular aspects only, and is not intended to be limiting, since the scope of the present disclosure will be limited only by the appended claims.

Accordingly, the terms defined immediately below are more fully defined by reference to the specification in its entirety.

### I. Definitions

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein. It is further noted that the claims can be drafted to exclude any optional element. As such, this statement is intended to serve as antecedent basis for use of such exclusive terminology as “solely,” “only” and the like in connection with the recitation of claim elements, or use of a negative limitation.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values that are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

Nucleotides are referred to by their commonly accepted single-letter codes. Unless otherwise indicated, nucleotide sequences are written left to right in 5′ to 3′ orientation. Nucleotides are referred to herein by their commonly known one-letter symbols recommended by the IUPAC-IUB Biochemical Nomenclature Commission. Accordingly, ‘a’ represents adenine, ‘c’ represents cytosine, ‘g’ represents guanine, ‘t’ represents thymine, and ‘u’ represents uracil.

About: The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

Administration: The terms “administration,” “administering,” and grammatical variants thereof refer to introducing a composition comprising a bicistronic polynucleotide disclosed herein, e.g., a polynucleotide, a vector, or a cell, into a subject via a pharmaceutically acceptable route. The introduction of a composition into a subject is by any suitable route, including orally, pulmonarily, intranasally, parenterally (intravenously, intra-arterially, intramuscularly, intraperitoneally, or subcutaneously), rectally, intralymphatically, intrathecally, intratumorally, periocularly or topically. Administration includes self-administration and the administration by another. A suitable route of administration allows the composition or the agent to perform its intended function. For example, if a suitable route is intravenous, the composition is administered by introducing the composition or agent into a vein of the subject. In some aspects, a cell is administered. In some aspects, the cell can be implanted.

Antibody: As use herein, the term “antibody” (Ab) shall include, without limitation, a glycoprotein immunoglobulin that binds specifically to an antigen and comprises at least two heavy (H) chains and two light (L) chains interconnected by disulfide bonds, or an antigen-binding portion thereof. Each H chain comprises a heavy chain variable region (abbreviated herein as V_{H}) and a heavy chain constant region. The heavy chain constant region comprises three constant domains, C_{H1}, C_{H2} and C_{H3}. Each light chain comprises a light chain variable region (abbreviated herein as V_{L}) and a light chain constant region. The light chain constant region comprises one constant domain, C_{L}. The V_{H} and V_{L} regions can be further subdivided into regions of hypervariability, termed complementarity determining regions (CDRs), interspersed with regions that are more conserved, termed framework regions (FRs). Each V_{H} and V_{L} comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system. Therefore, e.g., the term “anti-GD2 antibody” includes a full antibody having two heavy chains and two light chains that specifically binds to GD2 and antigen-binding portions of the full antibody. Non-limiting examples of the antigen-binding portions are shown elsewhere herein. In some aspects of the present disclosure, the anti-GD2 antibody is dinutuximab (UNITUXIN®) or an antigen-binding portion thereof.

A wide variety of recombinant antibody formats have been developed in the recent past, e.g. trivalent or tetravalent bispecific antibodies. Examples include the fusion of an IgG antibody format and single chain domains (for different formats see e.g. Coloma, M. J., et al, Nature Biotech 15 (1997), 159-163; WO 2001/077342; Morrison, S. L., Nature Biotech 25 (2007), 1233-1234; Holliger. P. et Al., Nature Biotech. 23 (2005), 1 126-1 136; Fischer, N., and Leger, O., Pathobiology 74 (2007), 3-14; Shen, J., et. al, J. Immunol. Methods 318 (2007), 65-74; Wu, C, et al., Nature Biotech. 25 (2007), 1290-1297). Bispecific antibodies include trivalent or tetravalent bispecific antibodies produced according to the methods disclosed in WO2009/080251; WO2009/080252; WO 2009/080253; WO2009/080254; WO2010/112193; WO2010/115589; WO2010/136172; WO2010/145792; WO2010/145793 and WO2011/117330, all of which are herein incorporated by reference in their entireties. A person of ordinary skill in the art would understand that higher order valencies can also be used.

Antigen: The term “antigen” refers to a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA.

Antigen binding portion: An “antigen-binding portion” of an antibody (also called an “antigen-binding fragment”) refers to one or more fragments of an antibody that retain the ability to bind specifically to the antigen bound by the whole antibody. It has been shown that the antigen-binding function of an antibody can be performed by fragments of a full-length antibody. Examples of binding fragments encompassed within the term “antigen-binding portion” of an antibody, e.g., an anti-GD2 antibody, include (i) a Fab fragment (fragment from papain cleavage) or a similar monovalent fragment consisting of the V_{L}, V_{H}, LC and CHI domains; (ii) a F(ab′)2 fragment (fragment from pepsin cleavage) or a similar bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546), which consists of a V_{H} domain; (vi) an isolated complementarity determining region (CDR) and (vii) a combination of two or more isolated CDRs which can optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

Approximately: As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

CAR: The term “Chimeric Antigen Receptor” or alternatively a “CAR” refers to a set of polypeptides, typically two in the simplest form, which when in an immune effector cell, provides the cell with specificity for a target cell, typically a cancer cell, and with intracellular signal generation. In some aspects, a CAR comprises at least an extracellular antigen-binding domain, a transmembrane domain and a cytoplasmic signaling domain (also referred to as “an intracellular signaling domain”) comprising a functional signaling domain derived from a stimulatory molecule and/or costimulatory molecule as defined below. In some aspects, the set of polypeptides are in the same polypeptide chain, e.g., comprise a chimeric fusion protein. In some aspects, the set of polypeptides are not contiguous with each other, e.g., are in different polypeptide chains. In some aspects, the set of polypeptides include a dimerization switch that, upon the presence of a dimerization molecule, can couple the polypeptides to one another, e.g., can couple an antigen-binding domain to an intracellular signaling domain. In some aspects, the stimulatory molecule of the CAR is the zeta chain associated with the T cell receptor complex (CD3 zeta). In some aspects, the cytoplasmic signaling domain comprises a primary signaling domain (e.g., a primary signaling domain of CD3 zeta). In some aspects, the cytoplasmic signaling domain further comprises one or more functional signaling domains derived from at least one costimulatory molecule defined below. In some aspects, the costimulatory molecule is chosen from the costimulatory molecules described herein, e.g., 4-1BB, CD27, and/or CD28.

In some aspects, the CAR comprises a chimeric fusion protein comprising an antigen-binding domain, a transmembrane domain and an intracellular signaling domain comprising a functional signaling domain derived from a stimulatory molecule, wherein the antigen-binding domain and the transmembrane domain are linked by a CAR spacer. In some aspects, the CAR comprises a chimeric fusion protein comprising an antigen-binding domain linked to a transmembrane domain via a CAR spacer and an intracellular signaling domain comprising a functional signaling domain derived from a costimulatory molecule and a functional signaling domain derived from a stimulatory molecule. In some aspects, the CAR comprises a chimeric fusion protein comprising an antigen-binding domain linked to a transmembrane domain via a CAR spacer and an intracellular signaling domain comprising two functional signaling domains derived from one or more costimulatory molecule(s) and a functional signaling domain derived from a stimulatory molecule. In some aspects, the CAR comprises a chimeric fusion protein comprising an antigen-binding domain linked to a transmembrane domain via a CAR spacer and an intracellular signaling domain comprising at least two functional signaling domains derived from one or more costimulatory molecule(s) and a functional signaling domain derived from a stimulatory molecule. In some aspects, the CAR comprises an optional leader sequence at the amino-terminus (N-terminus) of the CAR. In some aspects, the CAR further comprises a leader sequence at the N-terminus of the antigen-binding domain, wherein the leader sequence is optionally cleaved from the antigen-binding domain (e.g., a scFv) during cellular processing and localization of the CAR to the cellular membrane.

CDR: The term “complementarity determining region” or “CDR,” as used herein, refers to the sequences of amino acids within antibody variable regions that confer antigen specificity and binding affinity. For example, in general, there are three CDRs in each heavy chain variable region (e.g., HCDR1, HCDR2, and HCDR3) and three CDRs in each light chain variable region (LCDR1, LCDR2, and LCDR3). The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273, 927-948 (“Chothia” numbering scheme), or a combination thereof. Under the Kabat numbering scheme, in some embodiments, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under the Chothia numbering scheme, in some embodiments, the CDR amino acids in the VH are numbered 26-32 (HCDR1), 52-56 (HCDR2), and 95-102 (HCDR3); and the CDR amino acid residues in the VL are numbered 26-32 (LCDR1), 50-52 (LCDR2), and 91-96 (LCDR3). In a combined Kabat and Chothia numbering scheme, in some embodiments, the CDRs correspond to the amino acid residues that are part of a Kabat CDR, a Chothia CDR, or both. For instance, in some embodiments, the CDRs correspond to amino acid residues 26-35 (HCDR1), 50-65 (HCDR2), and 95-102 (HCDR3) in a VH, e.g., a mammalian VH, e.g., a human VH; and amino acid residues 24-34 (LCDR1), 50-56 (LCDR2), and 89-97 (LCDR3) in a VL, e.g., a mammalian VL, e.g., a human VL.

Complement: The term “complement” as used herein indicates a sequence that is complementary to a reference sequence. It is well known that complementarity is the base principle of DNA replication and transcription as it is a property shared between two DNA or RNA sequences, such that when they are aligned antiparallel to each other, the nucleotide bases at each position in the sequences will be complementary, much like looking in the mirror and seeing the reverse of things. Therefore, for example, the complement of a sequence of 5′ “ATGC” 3′ can be written as 3′ “TACG” 5′ or 5′ “GCAT” 3′. The terms “reverse complement”, “reverse complementary”, and “reverse complementarity” as used herein are interchangeable with the terms “complement”, “complementary”, and “complementarity.” In some aspects, the term “complementary” refers to 100% match or complementarity (i.e., fully complementary) to a contiguous nucleic acid sequence. In some aspects, the term “complementary” refers to at least about 80%, at least about 85%, at least about 90%, at least about 91%, at least about 92%, at least about 93%, at least about 94%, at least about 95%, at least about 96%, at least about 97%, at least about 98%, or at least about 99% match or complementarity to a contiguous nucleic acid sequence.

Complementary: The terms “complementary” and “complementarity” refer to two or more polynucleotides (i.e., each comprising a nucleobase sequence) that are related with one another by Watson-Crick base-pairing rules. For example, the nucleobase sequence “T-G-A (5′→3′),” is complementary to the nucleobase sequence “A-C-T (3′→5′).” Complementarity may be “partial,” in which less than all of the nucleobases of a given polynucleotide sequence are matched to the other polynucleotide sequence according to base pairing rules. For example, in some aspects, complementarity between a given polynucleotide sequence and the other polynucleotide sequence may be about 70%, about 75%, about 80%, about 85%, about 90% or about 95%. On the other hand, there may be “complete” or “perfect” (100%) complementarity between a given polynucleotide sequence and the other polynucleotide sequence to continue the example. The degree of complementarity between polynucleotide sequences has significant effects on the efficiency and strength of hybridization between the sequences.

Conserved: As used herein, the term “conserved” refers to nucleotides of a polynucleotide sequence which are those that occur unaltered in the same position of two or more sequences being compared. Nucleotides that are relatively conserved are those that are conserved amongst more related sequences than nucleotides appearing elsewhere in the sequences.

Corresponding to: The terms “corresponding to” and “corresponds to,” when referencing two separate nucleic acid or nucleotide sequences can be used to clarify regions of the sequences that correspond or are similar to each other based on homology and/or functionality, although the nucleotides of the specific sequences can be numbered differently. In addition, it is recognized that different numbering systems can be employed when characterizing a nucleic acid or nucleotide sequence. Further, it is recognized that the nucleic acid or nucleotide sequences of different variants of a nucleic acid can vary. As used herein, however, the regions of the variants that share nucleic acid or nucleotide sequence homology and/or functionality are deemed to “correspond” to one another.

Derived from: The terms “derived from” or “derivative” as used herein, refer to a component that is isolated from or made using a specified molecule, or information (e.g., a nucleic acid sequence) from the specified molecule. For example, a polynucleotide sequence that is derived from another polynucleotide sequence can include a polynucleotide sequence that is identical or substantially similar to the polynucleotide sequence it derives from. In the case of polynucleotides, the derived species can be obtained by, for example, naturally occurring mutagenesis, artificial directed mutagenesis, or artificial random mutagenesis. The mutagenesis used to derive polynucleotides can be intentionally directed or intentionally random, or a mixture of each. The mutagenesis of a polynucleotide to create a different polynucleotide derived from the first polynucleotide can be a random event (e.g., caused by polymerase infidelity) and the identification of the derived polynucleotide can be made by appropriate screening methods known in the art. In some aspects, a polynucleotide sequence that is derived from a first polynucleotide sequence has a sequence identity of at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identity to the first polynucleotide sequence, respectively, wherein the derived polynucleotide sequence retains the biological activity of the original polynucleotide.

Downstream Upstream: The term “downstream” refers to a nucleotide sequence that is located 3′ to a reference nucleotide sequence. In certain aspects, downstream nucleotide sequences relate to sequences that follow the starting point of transcription. For example, the translation initiation codon of a gene is located downstream of the start site of transcription. The term “upstream” refers to a nucleotide sequence that is located 5′ to a reference nucleotide sequence.

Encoding: The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a nucleotide sequence “encoding” an amino acid sequence,” e.g., a polynucleotide “encoding” a CAR of the present disclosure, includes all nucleotide sequences that are degenerate versions of each other and that encode the same amino acid sequence.

Epitope: As used herein, the term “epitope” refers to the moieties of an antigen that specifically interact with an antibody molecule. Such moieties, referred to herein as epitopic determinants, typically comprise, or are part of, elements such as amino acid side chains or sugar side chains. An epitopic determinate can be defined, e.g., by methods known in the art, e.g., by crystallography or by hydrogen-deuterium exchange. At least one or some of the moieties on the antibody molecule that specifically interact with an epitopic determinant are typically located in a CDR(s). Typically an epitope has specific three-dimensional structural characteristics. Typically an epitope has specific charge characteristics. Some epitopes are linear epitopes while others are conformational epitopes.

Expression: The term “expression” as used herein refers to a process by which a polynucleotide produces a gene product, for example, a RNA or a polypeptide. It includes, without limitation, transcription of the polynucleotide into messenger RNA (mRNA) and the translation of an mRNA into a polypeptide. Expression produces a “gene product.” As used herein, a gene product can be either a nucleic acid, e.g., a messenger RNA produced by transcription of a gene, or a polypeptide, which is translated from a transcript. In some aspects, the terms “expression” or “expresses” are used to refer to transcription and translation occurring within a cell. The level of expression of a product gene in a host cell can be determined based on either the amount of corresponding mRNA that is present in the cell or the amount of the protein encoded by the product gene that is produced by the cell, or both.

Fragment: As used herein, the term “fragment,” e.g., a B2M fragment, refers to a polynucleotide or polypeptide sequence that is shorter than the naturally occurring gene or protein. For example, in a polynucleotide fragment part of the polynucleotide sequence has been deleted in comparison to the naturally occurring polynucleotide. Likewise, in a polypeptide fragment part of the polypeptide sequence has been deleted in comparison to the naturally occurring polypeptide.

Functional fragment/non-functional fragment: As used herein, the term “functional fragment” refers to a polynucleotide fragment or polypeptide encoded by such polynucleotide fragment, e.g., a fragment of the B2M gene or a fragment of the B2M protein that retains at least partially the functionality of the intact gene or protein. Accordingly, in some aspects, a functional fragment of a B2M protein disclosed herein, retains the ability to associate with the major histocompatibility complex (MHC) class I heavy chain. Conversely, a “non-functional fragment” would lack one or more of the functional characteristics of the parent molecule.

Whether a fragment of a B2M promoter disclosed herein is a functional fragment or a non-functional fragment can be assessed by any art known methods without undue experimentation. In some aspects, the functional fragment retains, e.g., at least about 20%, at least about 30%, at least about 40%, at least about 50%, at least about 60%, at least about 70%, at least about 80%, at least about 90% or at least about 100% of the ability of an intact B2M protein to associate with the major histocompatibility complex (MHC) class I heavy chain. In some aspects, the non-functional fragment retains, e.g., less than about 20%, less than about 10%, less than about 5%, or fully lacks the ability of an intact B2M protein to associate with the major histocompatibility complex (MHC) class I heavy chain.

Functional variant non-functional variant: As used herein, the term “functional variant” refers to a polynucleotide variant or polypeptide (i.e., a mutant molecule having one or more substitutions, deletions, or insertion) encoded by such polynucleotide variant, e.g., a variant of the B2M gene or a variant of the B2M protein that retains at least partially the functionality of the intact gene or protein. Accordingly, in some aspects, a functional variant of a B2M protein disclosed herein, retains the ability to associate with the major histocompatibility complex (MHC) class I heavy chain. Conversely, a “non-functional variant” would lack one or more of the functional characteristics of the parent molecule. Whether a variant of a B2M promoter disclosed herein is a functional variant or a non-functional variant can be assessed by any art known methods without undue experimentation. In some aspects, the functional variant retains, e.g., at least about 20%, at least about 30%, at least about 40%, at least about 50%, at least about 60%, at least about 70%, at least about 80%, at least about 90% or at least about 100% of the ability of an intact B2M protein to associate with the major histocompatibility complex (MHC) class I heavy chain. In some aspects, the non-functional variant retains, e.g., less than about 20%, less than about 10%, less than about 5%, or fully lacks the ability of an intact B2M protein to associate with the major histocompatibility complex (MHC) class I heavy chain.

Gene: The terms “gene,” “coding sequence,” “encoding nucleic acid,” and grammatical variants thereof are used interchangeably in the present disclosure and refer to nucleic acids (RNA or DNA molecule) that comprise a nucleotide sequence which encodes a gene of interest, which is generally a protein, e.g., a therapeutic protein such as an antibody or a CAR. The coding sequence can further include initiation and termination signals operably linked to regulatory elements including a promoter and polyadenylation signal capable of directing expression in the cells of an individual or mammal to which the nucleic acid is administered. The coding sequence may be codon optimized.

Identical: In some aspects, two or more sequences are said to be “completely conserved” or “identical” if they are 100% identical to one another. In some aspects, two or more sequences are said to be “highly conserved” if they are at least 70% identical, at least 80% identical, at least 90% identical, or at least 95% identical to one another. In some aspects, two or more sequences are said to be “highly conserved” if they are about 70% identical, about 80% identical, about 90% identical, about 95% identical, about 98% identical, or about 99% identical to one another. In some aspects, two or more sequences are said to be “conserved” if they are at least 30% identical, at least 40% identical, at least 50% identical, at least 60% identical, at least 70% identical, at least 80% identical, at least 90% identical, or at least 95% identical to one another. In some aspects, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of a polynucleotide or polypeptide or may apply to a portion, region or feature thereof.

Identity: As used herein, the term “identity” refers to the overall monomer conservation between polymeric molecules, e.g., between polypeptide molecules or polynucleotide molecules (e.g. DNA molecules and/or RNA molecules). The term “identical” without any additional qualifiers, e.g., protein A is identical to protein B, implies the sequences are 100% identical (100% sequence identity). Describing two sequences as, e.g., “70% identical,” is equivalent to describing them as having, e.g., “70% sequence identity.”

Calculation of the percent identity of two polypeptide or polynucleotide sequences, for example, can be performed by aligning the two sequences for optimal comparison purposes (e.g., gaps can be introduced in one or both of a first and a second polypeptide or polynucleotide sequences for optimal alignment and non-identical sequences can be disregarded for comparison purposes). In certain aspects, the length of a sequence aligned for comparison purposes is at least about 30%, at least about 40%, at least about 50%, at least about 60%, at least about 70%, at least about 80%, at least about 90%, at least about 95%, or about 100% of the length of the reference sequence. The amino acids at corresponding amino acid positions, or bases in the case of polynucleotides, are then compared.

When a position in the first sequence is occupied by the same amino acid as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm.

Suitable software programs are available from various sources, and for alignment of both protein and nucleotide sequences. One suitable program to determine percent sequence identity is bl2seq, part of the BLAST suite of program available from the U.S. government's National Center for Biotechnology Information BLAST web site (blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at ebi.ac.uk/Tools/psa. In one particular aspects, sequence identity corresponds to the percentage of sequence identity of a global pairwise alignment determined using a program implementing the Needleman-Wunsch algorithm, e.g., Needle, available at ebi.ac.uk/Tools/psa/emboss_needle/.

Sequence alignments can be conducted using methods known in the art such as MAFFT, Clustal (ClustalW, Clustal X or Clustal Omega), MUSCLE, etc.

Different regions within a single polynucleotide or polypeptide target sequence that aligns with a polynucleotide or polypeptide reference sequence can each have their own percent sequence identity. It is noted that the percent of sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In certain aspects, the percentage identity (% ID) or of a first amino acid sequence (or nucleic acid sequence) to a second amino acid sequence (or nucleic acid sequence) is calculated as % ID=100×(Y/Z), where Y is the number of amino acid residues (or nucleobases) scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at tcoffee.org, and alternatively available, e.g., from the EBI. It will also be appreciated that the final alignment used to calculate percent sequence identity can be curated either automatically or manually.

Intracellular signaling domain: An “intracellular signaling domain,” as the term is used herein, refers to an intracellular portion of a molecule. The intracellular signaling domain can generate a signal that promotes an immune effector function of the CAR containing cell, e.g., a CAR T cell. Examples of immune effector function, e.g., in a CAR T cell, include cytolytic activity and helper activity, including the secretion of cytokines. In some aspects, the intracellular signal domain is the portion of the protein that transduces the effector function signal and directs the cell to perform a specialized function. While the entire intracellular signaling domain can be employed, in many cases it is not necessary to use the entire chain. To the extent that a truncated portion of the intracellular signaling domain is used, such truncated portion can be used in place of the intact chain as long as it transduces the effector function signal. The term intracellular signaling domain is thus meant to include any truncated portion of the intracellular signaling domain sufficient to transduce the effector function signal.

In an embodiment, the intracellular signaling domain can comprise a primary intracellular signaling domain. Exemplary primary intracellular signaling domains include those derived from the molecules responsible for primary stimulation, or antigen dependent simulation. In an embodiment, the intracellular signaling domain can comprise a costimulatory intracellular domain. Exemplary costimulatory intracellular signaling domains include those derived from molecules responsible for costimulatory signals, or antigen independent stimulation. For example, in the case of a CAR T, a primary intracellular signaling domain can comprise a cytoplasmic sequence of a T cell receptor, and a costimulatory intracellular signaling domain can comprise cytoplasmic sequence from co-receptor or costimulatory molecule.

A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3 zeta, FcR gamma, common FcR gamma (FCER1G), Fc gamma RIIa, FcR beta (Fc Epsilon Rib), CD3 gamma, CD3 delta, CD3 epsilon, CD22, CD79a, CD79b, CD278 (ICOS), FcεRI, CD66d, CD32, DAP10 and DAP12.

Mismatch: The terms “mismatch” or “mismatches” refer to one or more nucleobases (whether contiguous or separate) in an oligomer nucleobase sequence that are not matched to a target pre-mRNA according to base pairing rules. While perfect complementarity is often desired, some aspects can include one or more but preferably 6, 5, 4, 3, 2, or 1 mismatches with respect to the target pre-mRNA. Variations at any location within the oligomer are included. In certain aspects, antisense oligomers of the disclosure include variations in nucleobase sequence near the termini, variations in the interior, and if present are typically within about 6, 5, 4, 3, 2, or 1 subunits of the 5′ and/or 3′ terminus. In certain aspects, one, two, or three nucleobases can be removed and still provide on-target binding.

Nucleic acid: The terms “Nucleic acid,” “nucleic acid molecule,” “nucleotide sequence,” “polynucleotide,” and grammatical variants thereof are used interchangeably and refer to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Single stranded nucleic acid sequences refer to single-stranded DNA (ssDNA) or single-stranded RNA (ssRNA). Double stranded DNA-DNA, DNA-RNA, and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, supercoiled DNA and chromosomes. In discussing the structure of particular double-stranded DNA molecules, sequences can be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation. DNA includes, but is not limited to, cDNA, genomic DNA, plasmid DNA, synthetic DNA, and semi-synthetic DNA. A “nucleic acid composition” of the disclosure comprises one or more nucleic acids as described herein.

Nucleic acid sequence: The terms “nucleic acid sequence” and “nucleotide sequence” are used interchangeably and refer to a contiguous nucleic acid sequence. The sequence can be either single stranded or double stranded DNA or RNA, e.g., a gRNA.

Operably linked: “Operably linked” refers to a juxtaposition wherein the components so described are in a relationship permitting them to function in their intended manner. For instance, a promoter is operably linked to a coding sequence if the promoter affects its transcription or expression. For example, the different components in a CAR are operably linked. Likewise, the different components in a bicistronic polynucleotide or in a full donor construct disclosed herein are operably linked.

Pharmaceutically-acceptable: The terms “pharmaceutically-acceptable carrier,” “pharmaceutically-acceptable excipient,” and grammatical variations thereof, encompass any of the agents approved by a regulatory agency of the U.S. Federal government or listed in the U.S. Pharmacopeia for use in animals, including humans, as well as any carrier or diluent that does not cause the production of undesirable physiological effects to a degree that prohibits administration of the composition to a subject and does not abrogate the biological activity and properties of the administered compound. The term includes excipients and carriers that are useful in preparing a pharmaceutical composition and are generally safe, non-toxic, and desirable.

Pharmaceutical composition: As used herein, the term “pharmaceutical composition” refers to one or more of compounds mixed or intermingled with a therapeutic composition (e.g., a cell), or suspended in one or more other chemical components, such as pharmaceutically acceptable carriers and excipients. One purpose of a pharmaceutical composition is to facilitate administration of preparations of a medicament to a subject.

Polynucleotide: The term “polynucleotide” as used herein refers to polymers of nucleotides of any length, including ribonucleotides, deoxyribonucleotides, analogs thereof, or mixtures thereof. This term refers to the primary structure of the molecule. Thus, the term includes triple-, double- and single-stranded deoxyribonucleic acid (“DNA”), as well as triple-, double- and single-stranded ribonucleic acid (“RNA”).

More particularly, the term “polynucleotide” includes polydeoxyribonucleotides (containing 2-deoxy-D-ribose), polyribonucleotides (containing D-ribose), including tRNA, rRNA, hRNA, siRNA and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA. In some aspects of the present disclosure a polynucleotide can be, e.g., an RNA, e.g., mRNA, or DNA.

Polypeptide: The terms “polypeptide” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art. The term “polypeptide,” as used herein, refers to proteins, polypeptides, and peptides of any size, structure, or function. Polypeptides include gene products, naturally occurring polypeptides, synthetic polypeptides, homologs, orthologs, paralogs, fragments and other equivalents, variants, and analogs of the foregoing. A polypeptide can be a single polypeptide or can be a multi-molecular complex such as a dimer, trimer or tetramer. They can also comprise single chain or multichain polypeptides. Most commonly, disulfide linkages are found in multichain polypeptides. The term polypeptide can also apply to amino acid polymers in which one or more amino acid residues are an artificial chemical analogue of a corresponding naturally occurring amino acid. In some aspects, a “peptide” can be less than or equal to 50 amino acids long, e.g., about 5, about 10, about 15, about 20, about 25, about 30, about 35, about 40, about 45, or about 50 amino acids long.

Prevent: The terms “prevent,” “preventing,” and variants thereof as used herein, refer partially or completely delaying onset of an disease, disorder and/or condition; partially or completely delaying onset of one or more symptoms, features, or clinical manifestations of a particular disease, disorder, and/or condition; partially or completely delaying onset of one or more symptoms, features, or manifestations of a particular disease, disorder, and/or condition; partially or completely delaying progression from a particular disease, disorder and/or condition; and/or decreasing the risk of developing pathology associated with the disease, disorder, and/or condition. In some aspects, preventing an outcome is achieved through prophylactic treatment. As used herein, “prophylactic” refers to a therapeutic or course of action used to prevent the onset of a disease or condition, or to prevent or delay a symptom associated with a disease or condition. As used herein, a “prophylaxis” refers to a measure taken to maintain health and prevent or delay the onset of a disease or condition, or to prevent or delay symptoms associated with a disease or condition.

scFv: The term “scFv” refers to a fusion protein comprising at least one antibody portion comprising a variable region of a light chain and at least one antibody portion comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art. It is understood that percentage of similarity is contingent on the comparison scale used, i.e., whether the amino acids are compared, e.g., according to their evolutionary proximity, charge, volume, flexibility, polarity, hydrophobicity, aromaticity, isoelectric point, antigenicity, or combinations thereof.

Subject: The terms “subject,” “patient,” “individual,” and “host,” and variants thereof are used interchangeably herein and refer to any mammalian subject, including without limitation, humans, domestic animals (e.g., dogs, cats and the like), farm animals (e.g., cows, sheep, pigs, horses and the like), and laboratory animals (e.g., monkey, rats, mice, rabbits, guinea pigs and the like) for whom diagnosis, treatment, or therapy is desired, particularly humans. The methods described herein are applicable to both human therapy and veterinary applications. As used herein, the phrase “subject in need thereof” includes subjects, such as mammalian subjects, that would benefit from administration of a therapeutic agent, e.g., CAR-T cell comprising a bicistronic polynucleotide of the present disclosure.

Subsequence: As used herein, the term “subsequence” refers to a subset of contiguous nucleotides or amino acids in a sequence (either the physical sequence or its symbolic representation).

Therapeutically effective amount: As used herein the term “therapeutically effective amount” is the amount of a cell, e.g., an allogeneic T-cell or a pharmaceutical composition comprising such cell that is sufficient to a produce a desired therapeutic effect, pharmacologic and/or physiologic effect on a subject in need thereof. A therapeutically effective amount can be a “prophylactically effective amount” as prophylaxis can be considered therapy.

Treating: The terms “treat,” “treatment,” or “treating,” as used herein refers to, e.g., the reduction in severity of a disease or condition; the reduction in the duration of a disease course; the amelioration or elimination of one or more symptoms associated with a disease or condition; the provision of beneficial effects to a subject with a disease or condition, without necessarily curing the disease or condition. The term also include prophylaxis or prevention of a disease or condition or its symptoms thereof. In one aspect, the term “treating” or “treatment” means inducing an immune response in a subject against an antigen.

Modulate: As used herein, the terms “modulate,” “modify,” and grammatical variants thereof, generally refer when applied to a specific concentration, level, expression, function or behavior, to the ability to alter, by increasing or decreasing, e.g., directly or indirectly promoting/stimulating/up-regulating or interfering with/inhibiting/down-regulating the specific concentration, level, expression, function or behavior, such as, e.g., to act as an antagonist or agonist. In some instances a modulator can increase and/or decrease a certain concentration, level, activity or function relative to a control, or relative to the average level of activity that would generally be expected or relative to a control level of activity.

Site: The terms “target site” and “insertion site” refer to region of the chromosomal DNA of a cell comprising a recognition sequence for a nuclease, e.g., CRISPR. As used herein, the term “recognition sequence” refers to a DNA sequence that is bound and cleaved by an endonuclease. In the case of a CRISPR, the recognition sequence is the sequence, typically 16-24 base pairs, to which the guide RNA binds to direct Cas9 cleavage.

Vector: The terms “vector,” “expression vector,” “plasmid,” and grammatical variants thereof are used interchangeably in the present disclosure and refer to polynucleotide exogenous to the genome of a host cell, which is inserted into a particular location in the genome of a host cell (e.g., a T cell). In general, the plasmid comprises a plurality of elements such a recombination sites (e.g., homologous recombination sites and/or site-specific recombination sites), markers (e.g., detection markers and/or selection markers), one or more expression cassettes, or any combination thereof. In some aspects, the plasmid can be a linear plasmid. In other aspects, the plasmid can be a circular plasmid, e.g., an intact circular plasmid.

### II. Bicistronic Polynucleotides

The present disclosure provides a bicistronic polynucleotide encoding a (i) therapeutic agent (e.g., a CAR); and, (ii) an immune surveillance masking molecule (ISMM), wherein the ISMM comprises a non-functional beta-2-microglobulin (B2M), e.g., a non-functional fragment or non-functional variant of B2M, and a human leukocyte antigen (HLA), e.g., HLA-E, HLA-G, or a functional fragment or variant thereof. In some aspects, the ISMM comprises a polynucleotide encoding a B2M polypeptide non-functional fragment and an HLA-E polypeptide or a functional fragment or variant thereof.

As used herein, the terms “beta-2 microglobulin gene,” “B2M gene,” “B2M” and the like, are used interchangeably and refer to the human gene identified by NCBI Gene ID NO. 567 (Accession No. NG_012920.1), as well as naturally occurring variants of the human beta-2 microglobulin gene which encode a functional B2M polypeptide, and functional fragments and variants thereof.

As used herein, a “bicistronic” polynucleotide refers to a polynucleotide that upon transcription produces a single messenger RNA (mRNA) which comprises two coding sequences (i.e., cistrons) and encodes more than one product, e.g., two proteins. As used herein, a “polycistronic” polynucleotide refers to a polynucleotide that upon transcription produces a single messenger RNA (mRNA) which comprises more than two coding sequences (i.e., cistrons) and encodes more than two products, e.g., proteins. A bicistronic or polycistronic mRNA can comprise any element known in the art to allow for the translation of two or more genes from the same mRNA molecule including, but not limited to, an IRES element, a T2A element, a P2A element, an E2A element, and an F2A element.

In some aspects, the therapeutic agent can comprise an antibody or an antigen binding portion thereof, an enzyme, a receptor, a receptor ligand, a protein antibiotic, a fusion protein, a structural protein, a regulatory protein, a vaccine, a growth factor, a hormone, or a cytokine. In some aspects, the therapeutic agent can comprise one or more heterologous moieties, e.g., moieties to extend the plasma half-life of the biologic (e.g., non-structured polypeptides such as XTEN), moieties to facilitate transport across membranes or the brain blood barrier, moieties to increase or decrease the clearance rate, or moieties to direct the therapeutic agent to a particular cell or tissue type (i.e., a targeting moiety).

In some aspects, the therapeutic agent is an antibody or antigen binding portion thereof. In some aspects, the antibody or an antigen-binding fragment thereof specifically binds to an epitope on a tumor antigen. In some aspects, the tumor antigen comprises ROR1, HER2, AFP, TRAC, TCRB, BCMA, CLL-1, CS1, CD38, CD19, TSHR, CD123, CD22, CD30, CD70, CD171, CD33, EGFRVIII, GD2, GD3, Tn Ag, PSMA, ROR2, GPC1, GPC2, FLT3, FAP, TAG72, CD44v6, CEA, EPCAM, B7H3, KIT, IL-13Ra2, mesothelin, IL-11Ra, PSCA, PRSS21, VEGFR2, LewisY, CD24, PDGFR-beta, SSEA-4, CD20, folate receptor alpha, ERBB2 (Her2/neu), MUC1, MUC16, EGFR, NCAM, prostase, PAP, ELF2M, Ephrin B2, IGF-I receptor, CAIX, LMP2, gplOO, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-la, MAGE-Al, legumain, HPV E6, E7, MAGE AI, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MARTI, Ras mutant, hTERT, sarcoma translocation breakpoints, ML-IAP, ERG (TMPRSS2 ETS fusion gene), NA17, PAX3, Androgen receptor, Cyclin BI, MYCN, RhoC, TRP-2, CYPIB1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, CD2, CD3ε, CD4, CD5, CD7, the extracellular portion of the APRIL protein, and any combinations thereof.

In some aspects, the therapeutic agent is a CAR comprising an antigen-binding domain that specifically binds to an epitope on a tumor antigen on a target cell, e.g., an antigen disclosed above. In some aspects, the antigen-binding domain comprises an antibody or an antigen-binding portion thereof. In some aspects, the tumor antigen is disialoganglioside GD2.

GD2 is a disialogangioside overexpressed on many tumors. An antibody therapy targeting disialoganglioside GD2 (dinutuximab, UNITUXIN®) has been approved for pediatric neuroblastoma. The antibody, however, must be administered with several other expensive components. Dinutuximab is given in combination with granulocyte-macrophage colony stimulating factor (GM-CSF), interleukin-2 (IL-2) and 13-cis-retinoic acid (RA). Morphine is administered prior to, during, and for two hours after infusion of dinutuximab to manage the severe pain that this drug causes. An antihistamine and an anti-inflammatory are also given before, during, and after to manage the infusion reaction. These problems can be avoided by creating, as disclosed herein, anti-GD2 CAR T cells utilizing the dinutuximab antibody GD2-binding domain in a fashion that allows any donor T cells to become available for use in any patient, i.e., anti-GD2 therapy would be possible using off-the shelf allogeneic CAR T cells.

Accordingly, in some aspects, a polycistronic polypeptide of the present disclosure comprises a polynucleotide sequence encoding a CAR derived from dinutuximab. In some aspects, the antibody is a single-chain variable fragment (scFv) comprising the variable region of the heavy chain (VH) and the variable region of the light chain (VL) of an antibody, e.g., dinutuximab, UNITUXIN®.

In some aspects, the therapeutic agent is a CAR comprising a dinutuximab scFv comprising the protein sequence set forth in SEQ ID NO: 22. In some aspects, the antigen-binding domain cross-competes with dinutuximab. In some aspects, the antigen-binding domain binds to the same epitope as dinutuximab. In some aspects, the antigen-binding domain comprises VH CDR3 of dinutuximab. In some aspects, the antigen-binding domain further comprises a VH CDR1 and a VH CDR2. In some aspects, the VH CDR1 comprises the VH CDR1 of dinutiximab and/or the VH CDR2 comprises the VH CDR2 of dinutuximab. In some aspects, the antigen-binding domain further comprises a VL CDR1, VL CDR2, and/or VL CDR3. In some aspects, the VL CDR1 comprises the VL CDR1 of dinutuximab, the VL CDR2 comprises the VL CDR2 of dinutuximab, and/or the VL CDR3 comprises the VL CDR3 of dinutuximab.

In some aspects, the antigen-binding domain comprises
(i) VH CDR1 of SEQ ID NO: 59; VH CDR2 of SEQ ID NO: 63; and VH CDR3 of SEQ ID NO: 67; and/or VL CDR1 of SEQ ID NO: 71; VL CDR2 of SEQ ID NO: 75; and VL CDR3 of SEQ ID NO: 79; or,
(ii) VH CDR1 of SEQ ID NO: 60; VH CDR2 of SEQ ID NO: 64; and VH CDR3 of SEQ ID NO: 68; and/or VL CDR1 of SEQ ID NO: 72; VL CDR2 of SEQ ID NO: 76; and VL CDR3 of SEQ ID NO: 80; or,
(iii) VH CDR1 of SEQ ID NO: 61; VH CDR2 of SEQ ID NO: 65; and VH CDR3 of SEQ ID NO: 69; and/or VL CDR1 of SEQ ID NO: 73; VL CDR2 of SEQ ID NO: 77; and VL CDR3 of SEQ ID NO: 81; or,
(iv) VH CDR1 of SEQ ID NO: 62; VH CDR2 of SEQ ID NO: 66; and VH CDR3 of SEQ ID NO: 70; and/or VL CDR1 of SEQ ID NO: 74; VL CDR2 of SEQ ID NO: 78; and VL CDR3 of SEQ ID NO: 82; or,
(v) VH CDR1 of SEQ ID NO: 53; VH CDR2 of SEQ ID NO: 54; and VH CDR3 of SEQ ID NO: 55; and/or VL CDR1 of SEQ ID NO: 56; VL CDR2 of SEQ ID NO: 57; and VL CDR3 of SEQ ID NO: 58.

In some aspects, the antigen-binding domain comprises a VH and a VL, wherein the VH comprises the protein sequence set forth in SEQ ID NO: 44 and the VL comprises the protein sequence set forth in SEQ ID NO: 46. In some aspects, the antigen-binding domain comprises a VH comprising the protein sequence set forth in SEQ ID NO:44 and a VL comprising the protein sequence set forth in SEQ ID NO: 46. In some aspects, the VH and VL are connected via a linker. In some aspects, the VH and VL are connected in a VH-linker-VL or VL-linker-VH conformation. In some aspects, the linker in a Gly₄-Ser linker. In some aspects, the Gly₄-Ser linker comprises the sequence set forth in SEQ ID NO: 84.

In some aspects, the CAR construct is designed as a standard CAR, a split CAR, an off-switch CAR, an on-switch CAR, a first-generation CAR, a second-generation CAR, a third-generation CAR, or a fourth-generation CAR.

In some aspects, the antigen-binding domain is an lg NAR, a Fab, a Fab′, a F(ab)′2, a F(ab)′3, an Fv, a single chain variable fragment (scFv), a bis-scFv, a (scFv)2, a minibody, a diabody, a triabody, a tetrabody, an intrabody, a disulfide stabilized Fv protein (dsFv), a unibody, a nanobody, an affibody, a DARPin, a monobody, an adnectin, an alphabody, or a designed binder.

In some aspects, the CAR construct further comprises a transmembrane domain, an intracellular domain, and a spacer located between the antigen-binding domain and the transmembrane domain.

In some aspects, the intracellular domain of the CAR construct is a signaling domain derived from CD3zeta, FcR gamma, FcR beta, CD3 gamma, CD3 delta, CD3 epsilon, CD5, CD22, CD79a, CD79b, CD66d, or CD28. In some aspects, the intracellular domain of the CAR construct is derived from CD28. In some aspects, the transmembrane domain of the CAR construct is derived from CD28. In some aspects, the transmembrane domain is linked to the intracellular domain by a linker. In some aspects, the intracellular domain and transmembrane domain of the CAR construct are derived from the same molecule, e.g., CD28; thus, in some aspects, the transmembrane and intracellular domain are derived from CD28.

In some aspects, the transmembrane domain of the CAR is a transmembrane domain from a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 and CD154. In some aspects, the transmembrane domain can include at least the transmembrane region(s) of, e.g., KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2R beta, IL2R gamma, IL7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, NKG2C, or CD19.

In some aspects, the spacer of the CAR construct is a CD8alpha hinge. In some aspects, the spacer is derived from a hinge region of a human immunoglobulin. In some aspects, the human immunoglobulin hinge region is from IgA1, IgA2, IgD, IgE, IgG1, IgG2, IgG3, or IgM.

In some aspects, the CAR construct further comprises a co-stimulatory domain or a combination thereof. In some aspects, the co-stimulatory domain is derived from 2B4, HVEM, ICOS, LAG3, DAP10, DAP12, CD27, CD28, 4-1BB (CD137), OX40 (CD134), CD30, CD40, ICOS (CD278), glucocorticoid-induced tumor necrosis factor receptor (GITR), lymphocyte function-associated antigen-1 (LFA-1), CD2, CD7, LIGHT, NKG2C, B7-H3, CD3zeta, and combinations thereof. In some aspects, the co-stimulatory domain comprises a 4-1BB activation domain. In some aspects, the co-stimulatory domain comprises a CD3zeta activation domain. In some aspects, the co-stimulatory domain comprises a 4-1BB activation domain and a CD3zeta activation domain. In some aspects, the costimulatory domain comprises a functional signaling domain of a protein selected from the group consisting of OX40, CD2, CD27, CD28, CDS, ICAM-1, LFA-1 (CD11a/CD18), ICOS (CD278), and 4-1BB (CD137). In some aspects, the costimulatory domain comprises a functional signaling domain of a protein selected from the group consisting of MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

In some aspects, the CAR of the present disclosure further comprises a leader sequence. In some aspects, the CARs of the present disclosure are bispecific CARs. Accordingly, in some aspects, the polynucleotide encoding a CAR of the present disclosure encodes at least a polypeptide of a bispecific CAR (e.g., a CAR targeting a first antigen and second antigen).

In some aspects, the CAR construct comprises the nucleic acid sequence set forth in SEQ ID NO: 19. In some aspects, the CAR construct encodes the protein set forth in SEQ ID NO: 20.

In some aspects, the B2M non-functional polypeptide is a B2M fragment, for example, a non-functional fragment. In some aspects, the B2M non-functional polypeptide is a B2M variant, for example, a non-functional variant. In some aspects, the human leukocyte antigen (HLA) is HLA-E or HLA-G.

As used herein, “HLA-E” refers to a polynucleotide encoding the α heavy chain of HLA class I histocompatibility antigen, alpha E, also known as MHC class I antigen E. HLA-E is a heterodimer consisting of an α heavy chain and a light chain (β-2 microglobulin). The α heavy chain is approximately 45 kDa and anchored in the membrane. The HLA-E gene contains 8 exons. Exon one encodes the signal peptide, exons 2 and 3 encode the α1 and α2 domains, which both bind the peptide, exon 4 encodes the α3 domain, exon 5 encodes the transmembrane domain, and exons 6 and 7 encode the cytoplasmic tail. See Uniprot entry P13747, Entrez entry 3133, and RefSeq (mRNA) and (Protein) entries NM_005516 and NP_005507, which are herein incorporated by reference in their entireties.

As used herein, “HLA-G” refers to a polynucleotide encoding the α heavy chain of HLA-G histocompatibility antigen, class I, G, also known as human leukocyte antigen G. HLA-G is a heterodimer consisting of an α heavy chain and a light chain (beta-2 microglobulin). The α heavy chain is anchored in the membrane. HLA-G is coded for by 88 alleles. The heavy chain is approximately 45 kDa and its gene contains 8 exons. Exon one encodes the leader peptide, exons 2 and 3 encode the alpha1 and alpha2 domain, which both bind the peptide, exon 4 encodes the alpha3 domain, exon 5 encodes the transmembrane region, and exon 6 encodes the cytoplasmic tail. Exon 7 and 8 are not translated due to a stop codon present in exon 6. HLA-G can be expressed under at least seven isoforms through alternative splicing, called HLA-G1, HLA-G2, HLA-G3, HLA-G4, HLA-G5, HLA-G6, and HLA-G7. The protein can be both membrane-bound and soluble. HLA-G1 through G4 are membrane bound, accordingly, in some aspects, the bicistronic polynucleotides of the present disclosure comprise a nucleic acid. encoding HLA-G1, HLA-G2, HLA-G3, HLA-G4, HLA-G5, HLA-G6, a functional variant thereof, or a functional fragment thereof. See Uniprot entry P17693, Entrez entry 3135, and RefSeq (mRNA) and (Protein) entries NM_002127, NM_001363567, NM_001384280, NM_001384290, NP_002118, and NP 001350496, which are herein incorporated by reference in their entireties.

In some aspects, the HLA-E or HLA-G in a bicistronic polynucleotide can encode functional variants or functional fragments of their corresponding wild type forms. In some aspects, the HLA-E in a bicistronic polynucleotide can encode functional variants or functional fragments of the HLA-E portion of SEQ ID NO: 6.

In some aspects, the beta-2-microglobulin (B2M) polypeptide and the human leukocyte antigen (HLA) are connected by a linker, e.g., a flexible linker. In some aspects, the linker is a Gly₄-Ser linker (GSSS; SEQ ID NO: 83). In some aspects, the Gly₄-Ser linker comprises the sequence set forth in SEQ ID NO: 84 (GSSSGSSSGSSSGSSS).

In some aspects of the bicistronic polynucleotides of the present disclosure, (i) the nucleic acid sequence encoding the therapeutic agent and the nucleic acid sequence encoding the immune surveillance masking molecule (ISMM) are connect by a 2A element (e.g., a P2A element) sequence; or, (ii) the nucleic acid sequence encoding the therapeutic agent and the nucleic acid sequence encoding the immune surveillance masking molecule (ISMM) are connected by an Internal Ribosome Entry Site (IRES element). Thus, in some aspects, the cistrons are connected via an IRES element. IRES elements are RNA regions that recruit the 40S ribosomal subunit through cap-independent mechanisms. IRES elements often adopt complex RNA structures, which serve as the anchoring site for the ribosome guided by RNA-RNA and/or RNA-protein interactions.

In other aspects, the cistrons are connected via a 2A element. 2A self-cleaving peptides, or 2A peptides, are a class of 18-22 aa-long peptides, which can induce ribosomal skipping during translation of a protein in a cell. 2A elements are good candidates to replace IRES because of its small size and high cleavage efficiency between genes upstream and downstream of the 2A peptide. In some aspects, the 2A element is a P2A element (porcine teschovirus-1 2A). In other aspects, the 2A element is a F2A element (foot-and-mouth disease virus), E2A element (equine rhinitis A virus), or T2A element (thosea asigna virus 2A). In some aspects, the 2A element portion of a bicistronic polynucleotide comprises 2 or more 2A elements in tandem, wherein the 2A elements are selected from P2A, E2A, F2A, E2A, and T2A. In some aspects, the 2A element is P2A-T2A. In some aspects, the 2A element is 2A-T2A-E2A.

In some aspects, the nucleic acid sequence encoding the therapeutic agent comprises the sequence set forth in SEQ ID NO: 5. In some aspects, the nucleic acid sequence encoding the ISMM comprises the sequence set forth in SEQ ID NO: 6. In some aspects, the nucleic acid sequence encoding the therapeutic agent comprises the sequence set forth in SEQ ID NO: 5, the nucleic acid sequence encoding the ISMM comprises the sequence set forth in SEQ ID NO: 6. In some aspects, the bicistronic polynucleotide is selected from the group consisting of Bicistronic Construct 1 (BC1; SEQ ID NO: 7), Bicistronic Construct 2 (BC2; SEQ ID NO: 8), Bicistronic Construct 3 (BC3; SEQ ID NO: 9), Bicistronic Construct 4 (BC4; SEQ ID NO: 10), Bicistronic Construct 5 (BC5; SEQ ID NO: 9), Bicistronic Construct 6 (BC6; SEQ ID NO: 10), Bicistronic Construct 7 (BC7; SEQ ID NO: 11) or Bicistronic Construct 8 (BC8; SEQ ID NO: 12).

In some aspects, the bicistronic polynucleotide further comprises a 5′ sequence complementary to a B2M gene sequence upstream from an insertion site and a 3′ sequence complementary to a B2M gene sequence downstream from the insertion site. In some aspects, the 5′ sequence and 3′ sequence have the same length. In some aspects, the 5′ sequence and 3′ sequence are at least about 100 nucleotides, at least about 200 nucleotides, at least about 300 nucleotides, at least about 400 nucleotides, at least about 500 nucleotides, at least about 600 nucleotides, at least about 700 nucleotides, at least about 800 nucleotides, at least about 900 nucleotides, or at least about 1000 nucleotides in length.

In some aspects, the bicistronic polynucleotide is selected from the group consisting of Full Donor 1 (FD1; SEQ ID NO: 13), Full Donor 2 (FD2; SEQ ID NO: 14), Full Donor 3 (FD3; SEQ ID NO: 15), Full Donor 4 (FD4; SEQ ID NO: 16), Full Donor 5 (FD5; SEQ ID NO: 15), Full Donor 6 (FD6; SEQ ID NO: 16), Full Donor 7 (FD7; SEQ ID NO: 17), or Full Donor 8 (FD8; SEQ ID NO: 18).

In some aspects, the bicistronic polynucleotide is inserted in a site within B2M gene, wherein insertion of the bicistronic polynucleotide in the B2M gene inactivates (partially or completely) the B2M gene. In some aspects, the insertion of the bicistronic polynucleotide in the B2M gene is mediated by a nuclease. In some aspects, insertion of the bicistronic polynucleotide in the B2M gene is mediated by a CRISPR/Cas nuclease. In some aspects, the nuclease is CRISPR/Cas9. CRISPR and other genome edition alternatives that can be used in the methods of the present disclosure, e.g., zinc-finger nucleases (ZFNs), transcription activator-like effector nucleases (TALENs), and meganucleases (MNs) are discussed in more detail below.

In some aspects, the insertion site in the B2M gene is at an intron location. In some aspects, the insertion site in the B2M gene is at an intron-exon junction location. In some aspects, the insertion site in the B2M gene is at an exon location. In some aspects, the exon location is at exon 1. In some aspects, the insertion site is Site 1 (ACTCTCTCTTTCTGGCCTGG; SEQ ID NO: 1). In some aspects, the exon location is at exon 2. In some aspects, the insertion site is Site 2 (AGTCACATGGTTCACACGGC; SEQ ID NO:2) or Site 3 (CACAGCCCAAGATAGTTAAG; SEQ ID NO:3). In some aspects, the exon location is at exon 3. In some aspects, the insertion site is Site 4 (GAGACATGTAAGCAGCATCA; SEQ ID NO: 4).

### III. Vectors

The present disclosure also provides a vector comprising a bicistronic polynucleotide disclosed herein operably linked to a regulatory element, e.g., a promoter. In some aspects the promoter is the native promoter, e.g., the native B2M gene promoter.

In some aspects, the vector is a transfer vector. The term “transfer vector” refers to a composition of matter which comprises an isolated nucleic acid (e.g., a bicistronic polynucleotide of the present disclosure) and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

In some aspects, the vector is an expression vector. The term “expression vector” refers to a vector comprising a recombinant polynucleotide (e.g., a bicistronic polypeptide of the present disclosure) comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

In some aspects, the vector is a viral vector, a mammalian vector, or bacterial vector. In some aspects, the vector is a retroviral vector. In some aspects, the viral vector is selected from the group consisting of an adenoviral vector, a lentivirus, a Sendai virus vector, a baculoviral vector, an Epstein Barr viral vector, a papovaviral vector, a vaccinia viral vector, a herpes simplex viral vector, a hybrid vector, and an adeno associated virus (AAV) vector.

In some aspects, the adenoviral vector is a third generation adenoviral vector. ADEASY™ is by far the most popular method for creating adenoviral vector constructs. The system consists of two types of plasmids: shuttle (or transfer) vectors and adenoviral vectors. The transgene of interest is cloned into the shuttle vector, verified, and linearized with the restriction enzyme PmeI. This construct is then transformed into ADEASIER-1 cells, which are BJ5183 E. coli cells containing PADEASY™ PADEASY™ is a ˜33Kb adenoviral plasmid containing the adenoviral genes necessary for virus production. The shuttle vector and the adenoviral plasmid have matching left and right homology arms which facilitate homologous recombination of the transgene into the adenoviral plasmid. One can also co-transform standard BJ5183 with supercoiled PADEASY™ and the shuttle vector, but this method results in a higher background of non-recombinant adenoviral plasmids. Recombinant adenoviral plasmids are then verified for size and proper restriction digest patterns to determine that the transgene has been inserted into the adenoviral plasmid, and that other patterns of recombination have not occurred. Once verified, the recombinant plasmid is linearized with PacI to create a linear dsDNA construct flanked by ITRs. 293 or 911 cells are transfected with the linearized construct, and virus can be harvested about 7-10 days later. In addition to this method, other methods for creating adenoviral vector constructs known in the art at the time the present application was filed can be used to practice the methods disclosed herein.

In other aspects, the viral vector is a retroviral vector, e.g., a lentiviral vector (e.g., a third or fourth generation lentiviral vector). The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art.

Lentiviral vectors are usually created in a transient transfection system in which a cell line is transfected with three separate plasmid expression systems. These include the transfer vector plasmid (portions of the HIV provirus), the packaging plasmid or construct, and a plasmid with the heterologous envelop gene (env) of a different virus. The three plasmid components of the vector are put into a packaging cell which is then inserted into the HIV shell. The virus portions of the vector contain insert sequences so that the virus cannot replicate inside the cell system. Current third generation lentiviral vectors encode only three of the nine HIV-1 proteins (Gag, Pol, Rev), which are expressed from separate plasmids to avoid recombination-mediated generation of a replication-competent virus. In fourth generation lentiviral vectors, the retroviral genome has been further reduced (see, e.g., TAKARA® LENTI-X™ fourth-generation packaging systems).

In some aspects, non-viral methods can be used to deliver a nucleic acid comprising a bicistronic polynucleotide of the present disclosure into a cell or tissue of a subject. In some aspects, the non-viral method includes the use of a transposon. In some aspects, use of a non-viral method of delivery permits reprogramming of cells, e.g., T or NK cells, and direct infusion of the cells into the subject. In some aspects, a nucleic acid sequence comprising a bicistronic polynucleotide of the present disclosure can be inserted into the genome of a target cell (e.g., a T cell) or a host cell (e.g., a cell for recombinant expression of the CAR polypeptide) by using CRISPR/Cas systems and genome edition alternatives such as zinc-finger nucleases (ZFNs), transcription activator-like effector nucleases (TALENs), and meganucleases (MNs).

The present disclosure also provides a composition comprising a bicistronic polynucleotide disclosed herein, or a vector comprising a bicistronic polynucleotide disclosed herein. Also provided is a kit comprising (i) a bicistronic polynucleotide disclosed herein, (ii) a vector comprising a bicistronic polynucleotide disclosed herein, or (iii) a composition comprising a bicistronic polynucleotide disclosed herein or a vector comprising a bicistronic polynucleotide disclosed herein.

### IV. Cells

The present disclosure also provides a cell genetically modified to express a therapeutic agent (e.g., a CAR) and an ISMM, comprising (i) a bicistronic polynucleotide disclosed herein, (ii) a vector comprising a bicistronic polynucleotide disclosed herein, or (iii) a composition comprising a bicistronic polynucleotide disclosed herein or a vector comprising a bicistronic polynucleotide disclosed herein. In some aspects, the cell is a T cell, a natural killer (NK) cell, a natural killer T (NKT) cell, an ILC cell, a macrophage, or an antigen-presenting cell. In some aspects, the cell is allogeneic. The some aspects, the genetically modified cell is part of kit or article of manufacture.

In some aspects, the genetically modified cell disclosed herein has been transfected with (i) a bicistronic polynucleotide disclosed herein, (ii) a vector comprising a bicistronic polynucleotide disclosed herein, or (iii) a composition comprising a bicistronic polynucleotide disclosed herein or a vector comprising a bicistronic polynucleotide disclosed herein.

The term “transfected” (or equivalent terms “transformed” and “transduced”) refers to a process by which exogenous nucleic acid, e.g., a bicistronic polynucleotide or vector of the present disclosure, is transferred or introduced into the genome of the host cell, e.g., a T cell. A “transfected” cell is one that has been transfected, transformed or transduced with exogenous nucleic acid, e.g., a bicistronic polynucleotide or vector of the present disclosure. The terms cell or transfected cell include the primary subject cell and its progeny.

In some aspects, the cell (e.g., T cell) is transfected with a vector of the present disclosure, e.g., an AAV vector or a lentiviral vector comprising a bicistronic construct of the present disclosure. In some such aspects, the cell may stably express the therapeutic agent encoded by the polycistronic polynucleotide of the present disclosure (e.g., a CAR). In some aspects, the cell is an immune effector cell.

As used herein, term “immune effector cell” refers to a cell that is involved in an immune response, e.g., in the promotion of an immune effector response. “Immune effector function” or “immune effector response,” refer to function or response, e.g., of an immune effector cell, that enhances or promotes an immune attack of a target cell. For example, an immune effector function or response refers to a property of a T or NK cell that promotes killing or the inhibition of growth or proliferation, of a target cell. In the case of a T cell, primary stimulation and co-stimulation are examples of immune effector function or response.

The term “effector function” refers to a specialized function of a cell. Effector function of a T cell, for example, may be cytolytic activity or helper activity including the secretion of cytokines. The intracellular signaling domain of a CAR can generate a signal that promotes an immune effector function of the CAR containing cell, e.g., a CAR-T cell. Examples of immune effector function, e.g., in a CAR-T cell, include cytolytic activity and helper activity, including the secretion of cytokines.

In some specific aspects, the present disclosures provides allogeneic CAR-T cells comprising a specific bicistronic construct selected from bicistronic constructs BC1, BC2, BC3, BC4, BC5, BC6, BC7, or BC8, wherein the nucleic acid sequence encoding HLA-E has been replaced with a nucleic acid sequence encoding HLA-G.

In some aspects, the present disclosure provides an allogeneic CAR-T cell comprising a specific bicistronic construct selected from the group consisting of BC1, BC2, BC3, BC4, BC5, BC6, BC7 and BC8, wherein the nucleic acid sequence encoding a non-functional B2M fragment or variant has been replaced with a nucleic acid sequence encoding a non-functional TRAC fragment or variant, and wherein the bicistronic construct is inserted in the TRAC gene.

In some aspects, the present disclosure provides an allogeneic CAR-T cell comprising a specific bicistronic construct selected from bicistronic construct BC1, BC2, BC3, BC4, BC5, BC6, BC7 or BC8, wherein the nucleic acid sequence encoding a non-functional B2M fragment or variant has been replaced with a nucleic acid sequence encoding a non-functional CD52 fragment or variant, and wherein the bicistronic construct is inserted in the CD52 gene.

T cells can be obtained from various sources, including but not limited to peripheral blood mononuclear cells, bone marrow, lymph node tissue, cord blood, thymus tissue, tissue from a site of infection, ascites, pleural effusion, spleen tissue, and tumors.

### V. Pharmaceutical Compositions

The present disclosure also provides a pharmaceutical composition comprising a cell genetically modified to express a therapeutic agent and an ISMM, comprising (i) a bicistronic polynucleotide disclosed herein, (ii) a vector comprising a bicistronic polynucleotide disclosed herein, or (iii) a composition comprising a bicistronic polynucleotide disclosed herein or a vector comprising a bicistronic polynucleotide disclosed herein, which are suitable for administration to a subject.

The present disclosure also provides a pharmaceutical composition for treating cancer in a subject in need thereof wherein the pharmaceutical composition comprises a cell genetically modified to express a therapeutic agent and an ISMM, comprising (i) a bicistronic polynucleotide disclosed herein, (ii) a vector comprising a bicistronic polynucleotide disclosed herein, or (iii) a composition comprising a bicistronic polynucleotide disclosed herein or a vector comprising a bicistronic polynucleotide disclosed herein. In some aspects, the pharmaceutical composition is part of a kit or article of manufacture.

The present disclosure also provides a pharmaceutical composition for treating cancer in a subject in need thereof wherein the pharmaceutical composition comprises
(i) a bicistronic polynucleotide disclosed herein;
(ii) a vector comprising a bicistronic polynucleotide disclosed herein;
(iii) a composition comprising a bicistronic polynucleotide disclosed herein or a vector comprising a bicistronic polynucleotide disclosed herein; or,
(iv) a cell comprising (i), (ii) or (iii).

In some aspects, the pharmaceutical composition is part of a kit or article of manufacture. The pharmaceutical compositions generally comprise a bicistronic polynucleotide, vector, or cell of the present disclosure encoding a therapeutic agent (e.g., a CAR) and a pharmaceutically acceptable excipient or carrier in a form suitable for administration to a subject. Pharmaceutically acceptable excipients or carriers are determined in part by the particular composition being administered, as well as by the particular method used to administer the composition. There is a wide variety of suitable formulations of pharmaceutical compositions comprising therapeutic agent disclosed herein, e.g., a CAR (see, e.g., Remington's Pharmaceutical Sciences, Mack Publishing Co., Easton, Pa. 18th ed. (1990)). The pharmaceutical compositions are generally formulated sterile and in full compliance with all Good Manufacturing Practice (GMP) regulations of the U.S. Food and Drug Administration. In certain aspects, the pharmaceutical composition is co-administered with of one or more additional therapeutic agents, in a pharmaceutically acceptable carrier.

Acceptable carriers, excipients, or stabilizers are nontoxic to recipients (e.g., animals or humans) at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Examples of carriers or diluents include, but are not limited to, water, saline, Ringer's solutions, dextrose solution, and 5% human serum albumin. The use of such media and compounds for pharmaceutically active substances is well known in the art. Except insofar as any conventional media or compound is incompatible with the compositions of the present disclosure (e.g., polynucleotides, vectors, or cells), use thereof in the compositions is contemplated.

### VI. Methods

The present disclosure also provides a method of stimulating a T cell-mediated immune response to a target cell population or tissue in a subject, comprising administering an effective amount of the cell comprising a bicistronic polynucleotide disclosed herein to the subject. Also provided is a method of providing an anti-tumor immunity in a subject in need thereof, the method comprising administering to the subject an effective amount of a cell comprising a bicistronic polynucleotide disclosed herein. The present disclosure provides also methods of treating cancer in a subject in need thereof comprising administering to the subject an effective amount of a cell comprising a bicistronic polynucleotide disclosed herein. The present disclosure provides a method of preparing a population of cells for a therapy comprising transducing a population of cells isolated from a subject with a bicistronic polynucleotide, vector, or composition disclosed herein. In some aspects, the transduction comprises culturing the cell under suitable conditions. In some aspects, the therapy is allogeneic cell therapy.

In some aspects of these methods, the bicistronic polynucleotide is selected from the group consisting of Bicistronic Construct 1, Bicistronic Construct 2, Bicistronic Construct 3, Bicistronic Construct 4, Bicistronic Construct 5, Bicistronic Construct 6, Bicistronic Construct 7 and Bicistronic Construct 8. In some aspects, the bicistronic polynucleotide is selected from the group consisting of Full Donor 1, Full Donor 2, Full Donor 3, Full Donor 4, Full Donor 5, Full Donor 6, Full Donor 7, or Full Donor 8. In some aspects, the bicistronic polynucleotide is Bicistronic Construct 1. In some aspects, the bicistronic polynucleotide is Bicistronic Construct 2. In some aspects, the bicistronic polynucleotide is Bicistronic Construct 3. In some aspects, the bicistronic polynucleotide is Bicistronic Construct 4. In some aspects, the bicistronic polynucleotide is Bicistronic Construct 5. In some aspects, the bicistronic polynucleotide is Bicistronic Construct 6. In some aspects, the bicistronic polynucleotide is Bicistronic Construct 7. In some aspects, the bicistronic polynucleotide is Bicistronic Construct 8. In some aspects, the bicistronic polynucleotide is Full Donor 1. In some aspects, the bicistronic polynucleotide is Full Donor 2. In some aspects, the bicistronic polynucleotide is Full Donor 3. In some aspects, the bicistronic polynucleotide is Full Donor 4. In some aspects, the bicistronic polynucleotide is Full Donor 5. In some aspects, the bicistronic polynucleotide is Full Donor 6. In some aspects, the bicistronic polynucleotide is Full Donor 7. In some aspects, the bicistronic polynucleotide is Full Donor 8.

The present disclosure provides a method of generating a persisting population of genetically engineered cells (e.g., T cells) in a subject diagnosed, e.g., with cancer or an inflammatory disease, the method comprising administering to the subject a cell genetically engineered to express a bicistronic polynucleotide disclosed herein, e.g., a bicistronic construct encoding a CAR or any other therapeutic protein (e.g., an antibody). Also provided is a method of expanding a population of genetically engineered cells (e.g., T cells) in a subject diagnosed with cancer or an inflammatory disease, the method comprising administering to the subject a cell genetically engineered to express a bicistronic polynucleotide disclosed herein. In some aspects, the cell is a T cell. In some aspects, the T cell is an allogeneic T cell. In some aspects, the subject is a human subject.

The present disclosure also provides a method to generate an allogeneic cell for gene therapy comprising inserting a bicistronic construct comprising a nucleic acid encoding a therapeutic agent and a nucleic acid encoding an immune surveillance masking molecule (ISMM) in the beta-2-microglobulin (B2M) gene, wherein insertion of the bicistronic construct inactivates the B2M gene. In some aspects, the bicistronic construct gene can be inserted in other genes whose inactivation results in a diminished immune response when the genetically modified cell is administered to a subject who is not the cell donor. In some aspects, the nucleic acid encoding the ISMM comprises a nucleic acid encoding a human leukocyte antigen (HLA) selected from HLA-E or HLA-G, or a functional variant thereof. In some aspects, the gene therapy is CAR-T therapy.

In some aspects, the insertion site in the B2M gene is selected from Site 1 (ACTCTCTCTTTCTGGCCTGG; SEQ ID NO: 1); Site 2 (AGTCACATGGTTCACACGGC; SEQ ID NO:2); Site 3 (CACAGCCCAAGATAGTTAAG; SEQ ID NO:3); or Site 4 (GAGACATGTAAGCAGCATCA; SEQ ID NO: 4).

In some aspects, the insertion site comprises a sequence located about 10 nucleotides, about 20 nucleotides, about 30 nucleotides, about 40 nucleotides, about 50 nucleotides, about 60 nucleotides, about 70 nucleotides, about 80 nucleotides, about 90 nucleotides, about 100 nucleotides, about 110 nucleotides, about 120 nucleotides, about 130 nucleotides, about 140 nucleotides, about 150 nucleotides, about 160 nucleotides, about 170 nucleotides, about 180 nucleotides, about 190 nucleotides, about 200 nucleotides, about 225 nucleotides, about 250 nucleotides, about 275 nucleotides, about 300 nucleotides, about 325 nucleotides, about 350 nucleotides, about 375 nucleotides, about 400 nucleotides, about 425 nucleotides, about 450 nucleotides, about 475 nucleotides, or about 500 nucleotides upstream with respect to the 5′ end of Site 1, or the corresponding position in the complementary strand of the B2M gene.

In some aspects, the insertion site comprises a sequence located about 10 nucleotides, about 20 nucleotides, about 30 nucleotides, about 40 nucleotides, about 50 nucleotides, about 60 nucleotides, about 70 nucleotides, about 80 nucleotides, about 90 nucleotides, about 100 nucleotides, about 110 nucleotides, about 120 nucleotides, about 130 nucleotides, about 140 nucleotides, about 150 nucleotides, about 160 nucleotides, about 170 nucleotides, about 180 nucleotides, about 190 nucleotides, about 200 nucleotides, about 225 nucleotides, about 250 nucleotides, about 275 nucleotides, about 300 nucleotides, about 325 nucleotides, about 350 nucleotides, about 375 nucleotides, about 400 nucleotides, about 425 nucleotides, about 450 nucleotides, about 475 nucleotides, or about 500 nucleotides downstream with respect to the 3′ end of Site 1, or the corresponding position in the complementary strand of the B2M gene.

In some aspects, the insertion site comprises a sequence located about 10 nucleotides, about 20 nucleotides, about 30 nucleotides, about 40 nucleotides, about 50 nucleotides, about 60 nucleotides, about 70 nucleotides, about 80 nucleotides, about 90 nucleotides, about 100 nucleotides, about 110 nucleotides, about 120 nucleotides, about 130 nucleotides, about 140 nucleotides, about 150 nucleotides, about 160 nucleotides, about 170 nucleotides, about 180 nucleotides, about 190 nucleotides, about 200 nucleotides, about 225 nucleotides, about 450 nucleotides, about 475 nucleotides, or about 500 nucleotides upstream with respect to the 5′ end of Site 2, or the corresponding position in the complementary strand of the B2M gene.

In some aspects, the insertion site comprises a sequence located about 10 nucleotides, about 20 nucleotides, about 30 nucleotides, about 40 nucleotides, about 50 nucleotides, about 60 nucleotides, about 70 nucleotides, about 80 nucleotides, about 90 nucleotides, about 100 nucleotides, about 110 nucleotides, about 120 nucleotides, about 130 nucleotides, about 140 nucleotides, about 150 nucleotides, about 160 nucleotides, about 170 nucleotides, about 180 nucleotides, about 190 nucleotides, about 200 nucleotides, about 225 nucleotides, about 250 nucleotides, about 275 nucleotides, about 300 nucleotides, about 325 nucleotides, about 350 nucleotides, about 375 nucleotides, about 400 nucleotides, about 425 nucleotides, about 450 nucleotides, about 475 nucleotides, or about 500 nucleotides downstream with respect to the 3′ end of Site 2, or the corresponding position in the complementary strand of the B2M gene.

In some aspects, the insertion site comprises a sequence located about 10 nucleotides, about 20 nucleotides, about 30 nucleotides, about 40 nucleotides, about 50 nucleotides, about 60 nucleotides, about 70 nucleotides, about 80 nucleotides, about 90 nucleotides, about 100 nucleotides, about 110 nucleotides, about 120 nucleotides, about 130 nucleotides, about 140 nucleotides, about 150 nucleotides, about 160 nucleotides, about 170 nucleotides, about 180 nucleotides, about 190 nucleotides, about 200 nucleotides, about 225 nucleotides, about 250 nucleotides, about 275 nucleotides, about 300 nucleotides, about 325 nucleotides, about 350 nucleotides, about 375 nucleotides, about 400 nucleotides, about 425 nucleotides, about 450 nucleotides, about 475 nucleotides, or about 500 nucleotides upstream with respect to the 5′ end of Site 3, or the corresponding position in the complementary strand of the B2M gene.

In some aspects, the insertion site comprises a sequence located about 10 nucleotides, about 20 nucleotides, about 30 nucleotides, about 40 nucleotides, about 50 nucleotides, about 60 nucleotides, about 70 nucleotides, about 80 nucleotides, about 90 nucleotides, about 100 nucleotides, about 110 nucleotides, about 120 nucleotides, about 130 nucleotides, about 140 nucleotides, about 150 nucleotides, about 160 nucleotides, about 170 nucleotides, about 180 nucleotides, about 190 nucleotides, about 200 nucleotides, about 225 nucleotides, about 450 nucleotides, about 475 nucleotides, or about 500 nucleotides downstream with respect to the 3′ end of Site 3, or the corresponding position in the complementary strand of the B2M gene.

In some aspects, the insertion site comprises a sequence located about 10 nucleotides, about 20 nucleotides, about 30 nucleotides, about 40 nucleotides, about 50 nucleotides, about 60 nucleotides, about 70 nucleotides, about 80 nucleotides, about 90 nucleotides, about 100 nucleotides, about 110 nucleotides, about 120 nucleotides, about 130 nucleotides, about 140 nucleotides, about 150 nucleotides, about 160 nucleotides, about 170 nucleotides, about 180 nucleotides, about 190 nucleotides, about 200 nucleotides, about 225 nucleotides, about 250 nucleotides, about 275 nucleotides, about 300 nucleotides, about 325 nucleotides, about 350 nucleotides, about 375 nucleotides, about 400 nucleotides, about 425 nucleotides, about 450 nucleotides, about 475 nucleotides, or about 500 nucleotides upstream with respect to the 5′ end of Site 4, or the corresponding position in the complementary strand of the B2M gene.

In some aspects, the insertion site comprises a sequence located about 10 nucleotides, about 20 nucleotides, about 30 nucleotides, about 40 nucleotides, about 50 nucleotides, about 60 nucleotides, about 70 nucleotides, about 80 nucleotides, about 90 nucleotides, about 100 nucleotides, about 110 nucleotides, about 120 nucleotides, about 130 nucleotides, about 140 nucleotides, about 150 nucleotides, about 160 nucleotides, about 170 nucleotides, about 180 nucleotides, about 190 nucleotides, about 200 nucleotides, about 225 nucleotides, about 250 nucleotides, about 275 nucleotides, about 300 nucleotides, about 325 nucleotides, about 350 nucleotides, about 375 nucleotides, about 400 nucleotides, about 425 nucleotides, about 450 nucleotides, about 475 nucleotides, or about 500 nucleotides downstream with respect to the 3′ end of Site 4, or the corresponding position in the complementary strand of the B2M gene.

In some aspects, the insertion site is an insertion site that overlaps with Site 1, Site 2, Site 3, or Site 4. In some aspect, the insertion site is in a corresponding location on the complementary strand of the B2M gene.

Also provided is (i) a bicistronic polynucleotide disclosed herein; (ii) a vector comprising the bicistronic polynucleotide of (i); (iii) a composition comprising (i) or (ii); (iv) a kit comprising (i), (ii) or (iii); (v) a cell comprising any (i), (ii), or (iii); (vi) a composition comprising (v); (vii) a pharmaceutical composition comprising (i), (ii), (iii), (v), or (vi); or, (viii) a kit comprising (v), (vi) or (vii) for use as a medicament.

Also provided is (i) a bicistronic polynucleotide disclosed herein; (ii) a vector comprising the bicistronic polynucleotide of (i); (iii) a composition comprising (i) or (ii); (iv) a kit comprising (i), (ii) or (iii); (v) a cell comprising any (i), (ii), or (iii); (vi) a composition comprising (v); (vii) a pharmaceutical composition comprising (i), (ii), (iii), (v), or (vi); or, (viii) a kit comprising (v), (vi) or (vii), for use as a medicament for treating cancer or an inflammatory disease or condition in a subject in need thereof.

Also provided is the use of (i) a bicistronic polynucleotide disclosed herein; (ii) a vector comprising the bicistronic polynucleotide of (i); (iii) a composition comprising (i) or (ii); (iv) a kit comprising (i), (ii) or (iii); (v) a cell comprising any (i), (ii), or (iii); (vi) a composition comprising (v); (vii) a pharmaceutical composition comprising (i), (ii), (iii), (v), or (vi); or, (viii) a kit comprising (v), (vi) or (vii), for the manufacture of a medicament for treating cancer or an inflammatory disease or condition in a subject in need thereof.

### VII. Therapeutic Agents

The bicistronic polynucleotides disclosed herein comprise a gene of interest that encodes a therapeutic agent. In some aspects, the gene of interest comprises one or more polynucleotide sequences encoding a biologic, e.g., a CAR, an antibody or an antigen-binding portion thereof. In some aspects, the gene of interest comprises a polynucleotide sequence encoding a protein comprising amino acid sequences identical to or substantially similar to all or part of one of the following proteins: tumor necrosis factor (TNF), flt3 ligand (WO 94/28391), erythropoeitin, thrombopoeitin, calcitonin, IL-2, angiopoietin-2 (Maisonpierre et al. (1997), Science 277(5322): 55-60), ligand for receptor activator of NF-kappa B (RANKL, WO 01/36637), tumor necrosis factor (TNF)-related apoptosis-inducing ligand (TRAIL, WO 97/01633), thymic stroma-derived lymphopoietin, granulocyte colony stimulating factor, granulocyte-macrophage colony stimulating factor (GM-CSF, Australian Patent No. 588819), mast cell growth factor, stem cell growth factor (U.S. Pat. No. 6,204,363), epidermal growth factor, keratinocyte growth factor, megakaryote growth and development factor, RANTES, human fibrinogen-like 2 protein (FGL2; NCBI accession no. NM-00682; Rüegg and Pytela (1995), Gene 160:257-62) growth hormone, insulin, insulinotropin, insulin-like growth factors, parathyroid hormone, interferons including α-interferons, y-interferon, and consensus interferons (U.S. Pat. Nos. 4,695,623 and 4,897,471), nerve growth factor, brain-derived neurotrophic factor, synaptotagmin-like proteins (SLP 1-5), neurotrophin-3, glucagon, interleukins, colony stimulating factors, lymphotoxin-β, leukemia inhibitory factor, and oncostatin-M. See, e.g., Human Cytokines: Handbook for Basic and Clinical Research, all volumes (Aggarwal and Gutterman, eds. Blackwell Sciences, Cambridge, Mass., 1998); Growth Factors: A Practical Approach (Mckay and Leigh, eds., Oxford University Press Inc., New York, 1993); and The Cytokine Handbook, Vols. 1 and 2 (Thompson and Lotze eds., Academic Press, San Diego, Calif., 2003), which are herein incorporated by reference in their entireties.

In some aspects, the gene of interest comprises a polynucleotide sequence encoding a protein (e.g., a chimeric protein or a fusion protein) comprising all or part of the amino acid sequence of a receptor for any of the above-mentioned proteins, an antagonist to such a receptor or any of the above-mentioned proteins, and/or proteins substantially similar to such receptors or antagonists. These receptors and antagonists include: both forms of tumor necrosis factor receptor (TNFR, referred to as p55 and p75, U.S. Pat. Nos. 5,395,760 and 5,610,279), Interleukin-1 (IL-1) receptors (types I and II; EP U.S. Pat. Nos. 460,846, 4,968,607, and 5,767,064), IL-1 receptor antagonists (U.S. Pat. No. 6,337,072), IL-1 antagonists or inhibitors (U.S. Pat. Nos. 5,981,713, 6,096,728, and 5,075,222) IL-2 receptors, IL-4 receptors (EP Patent No. 0 367 566 and U.S. Pat. No. 5,856,296), IL-15 receptors, IL-17 receptors, IL-18 receptors, Fc receptors, granulocyte-macrophage colony stimulating factor receptor, granulocyte colony stimulating factor receptor, receptors for oncostatin-M and leukemia inhibitory factor, receptor activator of NF-kappa B (RANK, WO 01/36637 and U.S. Pat. No. 6,271,349), osteoprotegerin (U.S. Pat. No. 6,015,938), receptors for TRAIL (including TRAIL receptors 1, 2, 3, and 4), and receptors that comprise death domains, such as Fas or Apoptosis-Inducing Receptor (AIR).

In some aspects, a gene of interest comprises a polynucleotide sequence encoding a protein comprising all or part of the amino acid sequences of differentiation antigens (referred to as CD proteins) or their ligands or proteins substantially similar to either of these. Examples of such antigens include CD22, CD27, CD30, CD39, CD40, and ligands thereto (CD27 ligand, CD30 ligand, etc.). Several of the CD antigens are members of the TNF receptor family, which also includes 41BB and OX40. The ligands are often members of the TNF family, as are 41BB ligand and OX40 ligand.

In some aspects, a gene of interest comprises a polynucleotide sequence encoding enzymatically active proteins or their ligands can also be produced using the invention. Thus, in some aspects, the biscistronic constructs of the present disclosure can be used for gene replacement therapy, for example, to replace a defective copy of a gene encoding an enzyme (e.g., a clotting factor) with a fully functional copy of the gene. In this respect, the bicistronic construct may be inserted in the B2M gene, or it may be inserted in the locus of the defective gene. Examples of enzymatically active proteins include all or part of one of the following proteins or their ligands or a protein substantially similar to one of these: a disintegrin and metalloproteinase domain family members including TNF-alpha Converting Enzyme, kinases, glucocerebrosidase, superoxide dismutase, tissue plasminogen activator, Factor VIII, Factor IX, apolipoprotein E, apolipoprotein A-I, globins, an IL-2 antagonist, alpha-1 antitrypsin, ligands for any of the above-mentioned enzymes, and any other enzymes and their ligands.

In some aspects, a gene of interest comprises a polynucleotide sequence encoding an antibody or an antigen-binding portion thereof. Examples of antibodies include, but are not limited to, those that recognize any one or a combination of proteins including, but not limited to, the above-mentioned proteins and/or the following antigens: CD2, CD3, CD4, CD8, CD11a, CD14, CD18, CD20, CD22, CD23, CD25, CD33, CD40, CD44, CD52, CD80 (B7.1), CD86 (B7.2), CD147, IL-1α, IL-1β, IL-2, IL-3, IL-7, IL-4, IL-5, IL-8, IL-10, IL-2 receptor, IL-4 receptor, IL-6 receptor, IL-13 receptor, IL-18 receptor subunits, FGL2, PDGF-β and analogs thereof (see U.S. Pat. Nos. 5,272,064 and 5,149,792), VEGF, TGF, TGF-β2, TGF-β1, EGF receptor (see U.S. Pat. No. 6,235,883) VEGF receptor, hepatocyte growth factor, osteoprotegerin ligand, interferon gamma, B lymphocyte stimulator (BlyS, also known as BAFF, THANK, TALL-1, and zTNF4; see Do and Chen-Kiang (2002), Cytokine Growth Factor Rev. 13(1): 19-25), C5 complement, IgE, tumor antigen CA125, tumor antigen MUC1, PEM antigen, LCG (which is a gene product that is expressed in association with lung cancer), HER-2, HER-3, RAS (e.g., K-RAS), a tumor-associated glycoprotein TAG-72, the SK-1 antigen, tumor-associated epitopes that are present in elevated levels in the sera of patients with colon and/or pancreatic cancer, cancer-associated epitopes or proteins expressed on breast, colon, squamous cell, prostate, pancreatic, lung, and/or kidney cancer cells and/or on melanoma, glioma, or neuroblastoma cells, the necrotic core of a tumor, integrin alpha 4 beta 7, the integrin VLA-4, B2 integrins, TRAIL receptors 1, 2, 3, and 4, RANK, RANK ligand, TNF-α, the adhesion molecule VAP-1, epithelial cell adhesion molecule (EpCAM), intercellular adhesion molecule-3 (ICAM-3), leukointegrin adhesin, the platelet glycoprotein gp IIb/IIIa, cardiac myosin heavy chain, parathyroid hormone, rNAPc2 (which is an inhibitor of factor VIIa-tissue factor), MHC I, carcinoembryonic antigen (CEA), alpha-fetoprotein (AFP), tumor necrosis factor (TNF), CTLA-4 (which is a cytotoxic T lymphocyte-associated antigen), Fc-γ-1 receptor, HLA-DR 10 beta, HLA-DR antigen, sclerostin, L-selectin, Respiratory Syncitial Virus, human immunodeficiency virus (HIV), hepatitis B virus (HBV), Streptococcus mutans, and Staphylococcus aureus.

Specific examples of known antibodies or antigen binding portions there which can be produced using the methods of the invention include but are not limited to adalimumab, atezolizumab, bevacizumab, infliximab, abciximab, alemtuzumab, avelumab, bapineuzumab, basiliximab, belimumab, BMS-986156, briakinumab, canakinumab, cemiplimab, certolizumab pegol, cetuximab, conatumumab, CX-072, denosumab, durvalumab, eculizumab, gemtuzumab ozogamicin, golimumab, ibritumomab tiuxetan, INCAGN01876, ipilimumab, labetuzumab, LY300054, mapatumumab, matuzumab, mepolizumab, motavizumab, muromonab-CD3, natalizumab, nimotuzumab, nivolumab, ofatumumab, omalizumab, oregovomab, palivizumab, panitumumab, PDR001, oxelumab, pembrolizumab, pemtumomab, pertuzumab, ranibizumab, sintilimab, rituximab, rovelizumab, tislelizumab, tocilizumab, tositumomab, tremelimumab, trastuzumab, TRX518, ustekinumab, vedolizomab, vopratelimab. XmAb23104, zalutumumab, and zanolimumab.

In some aspects, the bicistronic polynucleotide can encode an anti-GITR such as TRX518, INCAGN01876, BMS-986156. In some aspects, the bicistronic polynucleotide can encode an anti-OX40 such as oxelumab. In some aspects, the bicistronic polynucleotide can encode an anti-ICOS (CD278) such as vopratelimab or XmAb23104 (anti-PD-1/anti-ICOS). In some aspects, the bicistronic polynucleotide can encode an anti-4-1BB (CD137) such as urelumab, utomilumab, INBRX-105 (anti-PD-L1/anti-4-1BB), or MCL A-145 (anti-PD-L1/anti-4-1BB). In some aspects, the bicistronic polynucleotide can encode an anti-PD-1 such as nivolumab, pembrolizumab, cemiplimab, PDR001, CBT-501, CX-188, TSR-042, XmAb20717 (anti PD-1/anti-CTLA-4), cetrelimab (JNJ-63723283), Gilvetmab (for canine veterinarian use), sintilimab (IBI308), tilselizumab, pidilizumab, prolgolimab (BCD 100), camrelizumab (SHR-1210), XmAb23104 (anti-PD-1/anti-ICOS), AK104 (anti-PD-1/anti-CTLA-4), MGD019 (anti-PD-1/anti-CTLA-4), XmAb20717 (anti-PD-1/anti-CTLA-4), MEDI5752 (anti-PD-1/anti-CTLA-4), MGD013 (anti-PD-1/anti-LAG3), RO7121661 (RG7769) (anti-PD-1/anti-TIM3), or IBI318 (anti-PD-1/undisclosed TAA). In some aspects, the bicistronic polynucleotide can encode an anti-PD-L2 such as AMP-224. In some aspects, the bicistronic polynucleotide can encode an anti-CTLA-4 such asipilimumab, XmAb20717 (anti PD-1/anti-CTLA-4), tremelimumab, AK104 (anti-PD-1/anti-CTLA-4), MGD019 (anti-PD-1/anti-CTLA-4), XmAb20717 (anti-PD-1/anti-CTLA-4), MEDI5752 (anti-PD-1/anti-CTLA-4), or KN046 (anti-PD-L1/anti-CTLA4). In some aspects, the bicistronic polynucleotide can encode an anti-VEGF such as varisacumab, bevacizumab, navicixizumab (OMP-305B83) (anti-DLL4/anti-VEGF), ABL101 (NOV1501)(anti-DLL4/anti-VEGF), ranibizumab, faricimab (anti-Ang2/anti-VEGFA), vanucizumab (anti-Ang2/Anti-VEGF), BI836880 (anti-Ang2/anti-VEGFA), or ABT165 (anti-DLL4/anti-VEGF). In some aspects, the bicistronic polynucleotide can encode an anti-VEGFR1 such as icrucumab (IMC-18F1). In some aspects, the bicistronic polynucleotide can encode an anti-VEGFR2 such as ramucirumab, alacizumab, or 33C3. In some aspects, the bicistronic polynucleotide can encode a CAR-T therapy such as IMM-3, axicabtagene ciloleucel, AUTO, Immunotox, sparX/ARC-T therapies, or BCMA CAR-T. In some aspects, the bicistronic polynucleotide can encode an angiopoietin 2 (Ang2) inhibitor such as vanucizumab (anti-Ang2/Anti-VEGF), faricimab (anti-Ang2/anti-VEGFA), nesvacumab, or BI836880 (anti-Ang2/anti-VEGFA). In some aspects, the bicistronic polynucleotide can encode an anti-FGFR1 such as BFKB8488A (RG7992) (anti-FGFR1/anti-KLB). In some aspects, the bicistronic polynucleotide can encode an anti-FGFR2 such as bemarituzumab (FPA144) or aprutumab (BAY 1179470). In some aspects, the bicistronic polynucleotide can encode an anti-DLL4/anti-VEGF such as navicixizumab (anti-DLL4/anti-VEGF), ABL101 (NOV1501) (anti-DLL4/anti-VEGF), or ABT165 (anti-DLL4/anti-VEGF). In some aspects, the bicistronic polynucleotide can encode an anti-Notch such as brontictuzumab or tarextumab. In some aspects, the bicistronic polynucleotide can encode an anti-DLL4 such as navicixizumab (anti-DLL4/anti-VEGF), ABL101 (NOV1501) (anti-DLL4/anti-VEGF), ABT165 (anti-DLL4/anti-VEGF), or demcizumab.

In some aspects, a gene of interest comprises a polynucleotide sequence encoding a recombinant fusion protein comprising, for example, any of the above-mentioned proteins or a functional fragment thereof (e.g., an enzymatically active portion or an antigen binding portion). For example, recombinant fusion proteins comprising one of the above-mentioned proteins or a functional portion thereof plus a multimerization domain, such as a leucine zipper, a coiled coil, an Fc portion of an immunoglobulin, or a substantially similar protein, can be produced using the methods of the invention. See e.g. WO94/10308; Lovejoy et al. (1993), Science 259:1288-1293; Harbury et al. (1993), Science 262:1401-05; Harbury et al. (1994), Nature 371:80-83; Håkansson et al. (1999), Structure 7:255-64.

Specifically included among such recombinant fusion proteins are proteins in which a portion of a receptor is fused to an Fc portion of an antibody such as etanercept (a p75 TNFR:Fc), abatacept, or belatacept (CTLA4:Fc). In some aspects, a gene of interest comprises a polynucleotide sequence encoding a marker, e.g., a screenable marker such as GFP or luciferase.

### VIII. Indications

In some aspects, the compositions disclosed herein, e.g., bicistronic polynucleotides of the present disclosure; vectors comprising the bicistronic polynucleotides; or cells comprising them can be used to prevent or treat a disease or condition, e.g., a proliferative disease such as a cancer or malignancy or a precancerous condition such as a myelodysplasia, a myelodysplastic syndrome or a preleukemia.

A “cancer” refers to a broad group of various proliferative diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. As used herein the term “proliferative” disorder or disease refers to unwanted cell proliferation of one or more subset of cells in a multicellular organism resulting in harm (i.e., discomfort or decreased life expectancy) to the multicellular organism. For example, as used herein, proliferative disorder or disease includes neoplastic disorders and other proliferative disorders. “Neoplastic,” as used herein, refers to any form of dysregulated or unregulated cell growth, whether malignant or benign, resulting in abnormal tissue growth. Thus, “neoplastic cells” include malignant and benign cells having dysregulated or unregulated cell growth. In some aspects, the cancer is a tumor. “Tumor,” as used herein, refers to all neoplastic cell growth and proliferation, whether malignant or benign, and all pre-cancerous and cancerous cells and tissues.

In some aspects, the disease is a solid or a liquid tumor. In some aspects, the cancer is a pancreatic cancer. In some aspects, the disease is a hematologic cancer. In some aspects, the hematologic cancer is a leukemia. In some aspects, the cancer is selected from the group consisting of one or more acute leukemias including but not limited to B-cell acute lymphoid leukemia (BALL), T-cell acute lymphoid leukemia (TALL), small lymphocytic leukemia (SLL), acute lymphoid leukemia (ALL) (e.g., relapsing and refractory ALL); one or more chronic leukemias including but not limited to chronic myelogenous leukemia (CML), and chronic lymphocytic leukemia (CLL). Additional hematologic cancers or conditions include, but are not limited to mantle cell lymphoma (MCL), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, Marginal zone lymphoma, multiple myeloma, myelodysplasia and myelodysplastic syndrome, non-Hodgkin lymphoma, Hodgkin lymphoma, plasmablastic lymphoma, plasmacytoid dendritic cell neoplasm, Waldenstrom macroglobulinemia, and preleukemia. Preleukemia encompasses a diverse collection of hematological conditions united by ineffective production (or dysplasia) of myeloid blood cells. In some aspects, the indication is an atypical and/or non-classical cancer, malignancy, precancerous condition or proliferative disease; and any combination thereof.

In some aspects, the disease is a lymphoma, e.g., MCL or Hodgkin lymphoma. In some aspects, the disease is leukemia, e.g., SLL, CLL and/or ALL. In some aspects, the disease associated with a tumor antigen, e.g., a tumor antigen described herein, is selected from a proliferative disease such as a cancer or malignancy or a precancerous condition such as a myelodysplasia, a myelodysplastic syndrome or a preleukemia, or is a non-cancer related indication associated with expression of a tumor antigen described herein. In some aspects, the disease associated with a tumor antigen described herein is a solid tumor, e.g., a solid tumor described herein, e.g., prostatic, colorectal, pancreatic, cervical, gastric, ovarian, head, or lung cancer.

In some aspects, the cancer is chosen from AML, ALL, B-ALL, T-ALL, B-cell prolymphocytic leukemia, chronic lymphocytic leukemia, CML, hairy cell leukemia, Hodgkin lymphoma, mast cell disorder, myelodysplastic syndrome, myeloproliferative neoplasm, plasma cell myeloma, plasmacytoid dendritic cell neoplasm, or a combination thereof.

In some aspects, the compositions disclosed herein (e.g., polynucleotides encoding CARs of the present disclosure, vectors comprising polynucleotides encoding CARs of the present disclosure, CARs of the present disclosure, or cells expressing CARs of the present disclosure, e.g., CAR-T cells) are used to reduce or decrease a size of a tumor or inhibit a tumor growth in a subject in need thereof. In some aspects, the tumor is a carcinoma (i.e., a cancer of epithelial origin). In some aspects, the tumor is, e.g., selected from the group consisting of gastric cancer, gastroesophageal junction cancer (GEJ), esophageal cancer, colorectal cancer, liver cancer (hepatocellular carcinoma, HCC), ovarian cancer, breast cancer, NSCLC, bladder cancer, lung cancer, pancreatic cancer, head and neck cancer, lymphoma, uterine cancer, renal or kidney cancer, biliary cancer, prostate cancer, testicular cancer, urethral cancer, penile cancer, thoracic cancer, rectal cancer, brain cancer (glioma and glioblastoma), cervical cancer, parotid cancer, larynx cancer, thyroid cancer, adenocarcinomas, neuroblastomas, melanoma, and Merkel Cell carcinoma.

A “cancer” or “cancer tissue” can include a tumor at various stages. In certain aspects, the cancer or tumor is stage 0, such that, e.g., the cancer or tumor is very early in development and has not metastasized. In some aspects, the cancer or tumor is stage I, such that, e.g., the cancer or tumor is relatively small in size, has not spread into nearby tissue, and has not metastasized. In other aspects, the cancer or tumor is stage II or stage III, such that, e.g., the cancer or tumor is larger than in stage 0 or stage I, and it has grown into neighboring tissues but it has not metastasized, except potentially to the lymph nodes. In other aspects, the cancer or tumor is stage IV, such that, e.g., the cancer or tumor has metastasized. Stage IV can also be referred to as advanced or metastatic cancer.

In some aspects, the cancer can include, but is not limited to, adrenal cortical cancer, advanced cancer, anal cancer, aplastic anemia, bileduct cancer, bladder cancer, bone cancer, bone metastasis, brain tumors, brain cancer, breast cancer, childhood cancer, cancer of unknown primary origin, Castleman disease, cervical cancer, colon/rectal cancer, endometrial cancer, esophagus cancer, Ewing family of tumors, eye cancer, gallbladder cancer, gastrointestinal carcinoid tumors, gastrointestinal stromal tumors, gestational trophoblastic disease, Hodgkin disease, Kaposi sarcoma, renal cell carcinoma, laryngeal and hypopharyngeal cancer, acute lymphocytic leukemia, acute myeloid leukemia, chronic lymphocytic leukemia, chronic myeloid leukemia, chronic myelomonocytic leukemia, liver cancer, non-small cell lung cancer, small cell lung cancer, lung carcinoid tumor, lymphoma of the skin, malignant mesothelioma, multiple myeloma, myelodysplastic syndrome, nasal cavity and paranasal sinus cancer, nasopharyngeal cancer, neuroblastoma, non-Hodgkin lymphoma, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma in adult soft tissue, basal and squamous cell skin cancer, melanoma, small intestine cancer, stomach cancer, testicular cancer, throat cancer, thymus cancer, thyroid cancer, uterine sarcoma, vaginal cancer, vulvar cancer, Waldenstrom macroglobulinemia, Wilms tumor and secondary cancers caused by cancer treatment.

In some aspects, the tumor is a solid tumor. A “solid tumor” includes, but is not limited to, sarcoma, melanoma, carcinoma, or other solid tumor cancer. “Sarcoma” refers to a tumor which is made up of a substance like the embryonic connective tissue and is generally composed of closely packed cells embedded in a fibrillar or homogeneous substance. Sarcomas include, but are not limited to, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” refers to a tumor arising from the melanocytic system of the skin and other organs. Melanomas include, for example, acra-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, metastatic melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas include, e.g., acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidernoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma viflosum.

Additional cancers that can be treated with the compositions disclosed herein (e.g., polynucleotides encoding CARs of the present disclosure, vectors comprising polynucleotides encoding CARs of the present disclosure, CARs of the present disclosure, or cells expressing CARs of the present disclosure, e.g., CAR-T cells) include, e.g., Leukemia, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, urinary bladder cancer, premalignant skin lesions, testicular cancer, lymphomas, thyroid cancer, papillary thyroid cancer, neuroblastoma, neuroendocrine cancer, esophageal cancer, genitourinary tract cancer, malignant hypercalcemia, cervical cancer, endometrial cancer, adrenal cortical cancer, prostate cancer, Müllerian cancer, ovarian cancer, peritoneal cancer, fallopian tube cancer, or uterine papillary serous carcinoma.

Other diseases, disorders, and conditions that can be treated with the compositions disclosed herein are, for example, inflammatory diseases or conditions, neurodegenerative disorders, enzyme deficiencies, hormonal deficiencies, clotting disorders, or infections.

### IX. Nucleases

The bicistronic polypeptides of the present disclosure can be inserted at a specific location (site) of a target gene using any methods known in the art. In some aspects, the bicistronic polypeptides of the present disclosure are inserted in the gene using nucleases. As used herein, the term “nuclease” refers to an enzyme which possesses catalytic activity for DNA cleavage. In some aspects, a nuclease agent can promote homologous recombination between a bicistronic construct (BC1-BC8) or full donor construct (FD1-FD8) disclosed herein and a gene, e.g., the B2M gene. In some aspects, the bicistronic construct to be integrated in the genome of a host cell line (e.g., a T cell) contains regions of homology adjacent to a sequence (e.g., Site 1 to Site 4) targeted by a nuclease, e.g., a CRISPR/Cas nuclease.

The size of the recognition site for the nuclease mediating homologous recombination can vary, and includes, for example, recognition sites that are at least about 4, at least about 6, at least about 8, at least about 10, at least about 12, at least about 14, at least about 16, at least about 18, at least about 19, at least about 20, at least about 21, at least about 22, at least about 23, at least about 24, at least about 25, at least about 26, at least about 27, at least about 28, at least about 29, at least about 30, at least about 31, at least about 32, at least about 33, at least about 34, at least about 35, at least about 36, at least about 37, at least about 38, at least about 39, at least about 40, at least about 41, at least about 42, at least about 43, at least about 44, at least about 45, at least about 46, at least about 47, at least about 48, at least about 49, at least about 50, at least about 51, at least about 52, at least about 53, at least about 54, at least about 55, at least about 56, at least about 57, at least about 58, at least about 59, at least about 60, at least about 61, at least about 62, at least about 63, at least about 64, at least about 65, at least about 66, at least about 67, at least about 68, at least about 69, at least about 70, at least about 80, at least about 90, at least about 100, at least about 110, at least about 120, at least about 130, at least about 140, at least about 150, at least about 160, at least about 170, at least about 180, at least about 190, at least about 200, at least about 210, at least about 220, at least about 230, at least about 240, at least about 250, at least about 260, at least about 270, at least about 280, at least about 290, at least about 300, at least about 310, at least about 320, at least about 330, at least about 340, at least about 350, at least about 360, at least about 370, at least about 380, at least about 390, at least about 400, at least about 410, at least about 420, at least about 430, at least about 440, at least about 450, at least about 460, at least about 470, at least about 480, at least about 490, at least about 500, at least about 510, at least about 520, at least about 530, at least about 540, at least about 550, at least about 560, at least about 570, at least about 580, at least about 590, at least about 600, at least about 610, at least about 620, at least about 630, at least about 640, at least about 650, at least about 660, at least about 670, at least about 680, at least about 690, at least about 700, at least about 710, at least about 720, at least about 730, at least about 740, at least about 750, at least about 760, at least about 770, at least about 780, at least about 790, at least about 800, at least about 810, at least about 820, at least about 830, at least about 840, at least about 850, at least about 860, at least about 870, at least about 880, at least about 890, at least about 900, at least about 910, at least about 920, at least about 930, at least about 940, at least about 950, at least about 960, at least about 970, at least about 980, at least about 990, at least about 1000 or more nucleotides in length.

The size of the recognition site for a nuclease mediating homologous recombination can vary, and includes, for example, recognition sites that are about 4, about 6, about 8, about 10, about 12, about 14, about 16, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, about 400, about 410, about 420, about 430, about 440, about 450, about 460, about 470, about 480, about 490, about 500, about 510, about 520, about 530, about 540, about 550, about 560, about 570, about 580, about 590, about 600, about 610, about 620, about 630, about 640, about 650, about 660, about 670, about 680, about 690, about 700, about 710, about 720, about 730, about 740, about 750, about 760, about 770, about 780, about 790, about 800, about 810, about 820, about 830, about 840, about 850, about 860, about 870, about 880, about 890, about 900, about 910, about 920, about 930, about 940, about 950, about 960, about 970, about 980, about 990, about 1000, or more nucleotides in length.

In one aspect, each monomer of the nuclease agent recognizes a recognition site of at least 9 nucleotides. In other aspects, the recognition site is from about 9 to about 12 nucleotides in length, from about 12 to about 15 nucleotides in length, from about 15 to about 18 nucleotides in length, or from about 18 to about 21 nucleotides in length, and any combination of such subranges (e.g., 9-18 nucleotides).

The recognition site can be palindromic, that is, the sequence on one strand reads the same in the opposite direction on the complementary strand. It is recognized that a given nuclease agent can bind the recognition site and cleave that binding site or alternatively, the nuclease agent can bind to a sequence that is the different from the recognition site. Moreover, the term recognition site comprises both the nuclease agent binding site and the nick/cleavage site irrespective whether the nick/cleavage site is within or outside the nuclease agent binding site. In another variation, the cleavage by the nuclease agent can occur at nucleotide positions immediately opposite each other to produce a blunt end cut or, in other cases, the incisions can be staggered to produce single-stranded overhangs, also called “sticky ends,” which can be either 5′ overhangs, or 3′ overhangs.

Any nuclease agent that induces a nick or double-strand break into a desired recognition site can be used in the methods and compositions disclosed herein. A naturally occurring or native nuclease agent can be employed so long as the nuclease agent induces a nick or double-strand break in a desired recognition site. Alternatively, a modified or engineered nuclease agent can be employed. An “engineered nuclease agent” comprises a nuclease that is engineered (modified or derived) from its native form to specifically recognize and induce a nick or double-strand break in the desired recognition site. Thus, an engineered nuclease agent can be derived from a native, naturally occurring nuclease agent or it can be artificially created or synthesized. The modification of the nuclease agent can be as little as one amino acid in a protein cleavage agent or one nucleotide in a nucleic acid cleavage agent. In some aspects, the engineered nuclease induces a nick or double-strand break in a recognition site, wherein the recognition site was not a sequence that would have been recognized by a native (non-engineered or non-modified) nuclease agent. Producing a nick or double-strand break in a recognition site or other DNA can be referred to herein as “cutting” or “cleaving” the recognition site or other DNA.

The nuclease agent may be introduced into the cell by any means known in the art. The polypeptide encoding the nuclease agent may be directly introduced into the cell. Alternatively, a polynucleotide encoding the nuclease agent can be introduced into the cell. When a polynucleotide encoding the nuclease agent is introduced into the cell, the nuclease agent can be transiently, conditionally, or constitutively expressed within the cell. Thus, the polynucleotide encoding the nuclease agent can be contained in an expression cassette and be operably linked to a conditional promoter, an inducible promoter, a constitutive promoter, or a tissue-specific promoter. Such promoters of interest are discussed in further detail elsewhere herein. Alternatively, the nuclease agent is introduced into the cell as an mRNA encoding or comprising a nuclease agent.

Active variants and fragments of nuclease agents (i.e., an engineered nuclease agent) are also provided. Such active variants can comprise at least 65%, 70%, 75%, 80%, 85%, 90%, 91%, 92%, 93%, 94%, 95%, 96%, 97%, 98%, 99% or more sequence identity to the native nuclease agent, wherein the active variants retain the ability to cut at a desired recognition site and hence retain nick or double-strand-break-inducing activity. For example, any of the nuclease agents described herein can be modified from a native endonuclease sequence and designed to recognize and induce a nick or double-strand break at a recognition site that was not recognized by the native nuclease agent. Thus in some aspects, the engineered nuclease has a specificity to induce a nick or double-strand break at a recognition site that is different from the corresponding native nuclease agent recognition site. Assays for nick or double-strand-break-inducing activity are known and generally measure the overall activity and specificity of the endonuclease on DNA substrates containing the recognition site.

When the nuclease agent is provided to the cell through the introduction of a polynucleotide encoding the nuclease agent, such a polynucleotide encoding a nuclease agent can be modified to substitute codons having a higher frequency of usage in the cell of interest, as compared to the naturally occurring polynucleotide sequence encoding the nuclease agent. For example, the polynucleotide encoding the nuclease agent can be modified to substitute codons having a higher frequency of usage in a given prokaryotic or eukaryotic cell of interest, including a bacterial cell, a yeast cell, a human cell, a non-human cell, a non-rat eukaryotic cell, a mammalian cell, a rodent cell, a non-rat rodent cell, a mouse cell, a rat cell, a hamster cell or any other host cell of interest, as compared to the naturally occurring polynucleotide sequence.

In some aspects of the present disclosure, the homologous recombination is mediated by a CRISPR/Cas system, a TALEN system, a ZFN system, a mega nuclease, or a restriction endonuclease.

### CRISPR/Cas

In some aspects, the nuclease agent employed in the various methods and compositions disclosed herein can comprise a CRISPR/Cas system. Such systems can employ, for example, a Cas9 nuclease, which in some instances, is codon-optimized for the desired cell type in which it is to be expressed. Such systems can also employ a guide RNA (gRNA) that comprises two separate molecules. An exemplary two-molecule gRNA comprises a crRNA-like (“CRISPR RNA” or “targeter-RNA” or “crRNA” or “crRNA repeat”) molecule and a corresponding tracrRNA-like (“trans-acting CRISPR RNA” or “activator-RNA” or “tracrRNA” or “scaffold”) molecule.

A crRNA comprises both the DNA-targeting segment (single stranded) of the gRNA and a stretch of nucleotides that forms one-half of a double stranded RNA (dsRNA) duplex of the protein-binding segment of the gRNA. A corresponding tracrRNA (activator-RNA) comprises a stretch of nucleotides that forms the other half of the dsRNA duplex of the protein-binding segment of the gRNA. Thus, a stretch of nucleotides of a crRNA are complementary to and hybridize with a stretch of nucleotides of a tracrRNA to form the dsRNA duplex of the protein-binding domain of the gRNA. As such, each crRNA can be said to have a corresponding tracrRNA. The crRNA additionally provides the single stranded DNA-targeting segment. Accordingly, a gRNA comprises a sequence that hybridizes to a target sequence, and a tracrRNA. Thus, a crRNA and a tracrRNA (as a corresponding pair) hybridize to form a gRNA. If used for modification within a cell, the exact sequence and/or length of a given crRNA or tracrRNA molecule can be designed to be specific to the species in which the RNA molecules will be used.

Naturally occurring genes encoding the three elements (Cas9, tracrRNA and crRNA) are typically organized in operon(s). Naturally occurring CRISPR RNAs differ depending on the Cas9 system and organism but often contain a targeting segment of between 21 to 72 nucleotides length, flanked by two direct repeats (DR) of a length of between 21 to 46 nucleotides (see, e.g., WO2014/131833). In the case of S. pyogenes, the DRs are 36 nucleotides long and the targeting segment is 30 nucleotides long. The 3′ located DR is complementary to and hybridizes with the corresponding tracrRNA, which in turn binds to the Cas9 protein.

Alternatively, the system further employs a fused crRNA-tracrRNA construct (i.e., a single transcript) that functions with the codon-optimized Cas9. This single RNA is often referred to as a guide RNA or gRNA. Within a gRNA, the crRNA portion is identified as the “target sequence” for the given recognition site and the tracrRNA is often referred to as the “scaffold.” Briefly, a short DNA fragment containing the target sequence is inserted into a guide RNA expression plasmid. The gRNA expression plasmid comprises the target sequence (in some aspects around 20 nucleotides), a form of the tracrRNA sequence (the scaffold) as well as a suitable promoter that is active in the cell and necessary elements for proper processing in eukaryotic cells. Many of the systems rely on custom, complementary oligonucleotides that are annealed to form a double stranded DNA and then cloned into the gRNA expression plasmid.

The gRNA expression cassette and the Cas9 expression cassette are then introduced into the cell. See, for example, Mali P et al. (2013) Science 2013 Feb. 15; 339(6121): 823-6; Jinek M et al. Science 2012 Aug. 17; 337(6096):816-21; Hwang W Y et al. Nat Biotechnol 2013 March; 31(3):227-9; Jiang W et al. Nat Biotechnol 2013 March; 31(3):233-9; and Cong L et al. Science 2013 Feb. 15; 339(6121):819-23, each of which is herein incorporated by reference. See also, for example, WO/2013/176772A1, WO/2014/065596A1, WO/2014/089290A1, WO/2014/093622A2, WO/2014/099750A2, and WO/2013142578A1, each of which is herein incorporated by reference.

In some aspects, the Cas9 nuclease can be provided in the form of a protein. In some aspects, the Cas9 protein can be provided in the form of a complex with the gRNA. In other aspects, the Cas9 nuclease can be provided in the form of a nucleic acid encoding the protein. The nucleic acid encoding the Cas9 nuclease can be RNA (e.g., messenger RNA (mRNA)) or DNA. In some aspects, the gRNA can be provided in the form of RNA. In other aspects, the gRNA can be provided in the form of DNA encoding the RNA. In some aspects, the gRNA can be provided in the form of separate crRNA and tracrRNA molecules, or separate DNA molecules encoding the crRNA and tracrRNA, respectively.

In some aspects, the gRNA comprises a third nucleic acid sequence encoding a Clustered Regularly Interspaced Short Palindromic Repeats (CRISPR) RNA (crRNA) and a trans-activating CRISPR RNA (tracrRNA). In one aspect, the Cas protein is a type I Cas protein. In one aspect, the Cas protein is a type II Cas protein. In one aspect, the type II Cas protein is Cas9. In one aspect, the type II Cas, e.g., Cas9, is a human codon-optimized Cas.

In certain aspects, the Cas protein is a “nickase” that can create single strand breaks (i.e., “nicks”) at the target site without cutting both strands of double stranded DNA (dsDNA). Cas9, for example, comprises two nuclease domains—a RuvC-like nuclease domain and an HNH-like nuclease domain—which are responsible for cleavage of opposite DNA strands. Mutation in either of these domains can create a nickase. Examples of mutations creating nickases can be found, for example, WO/2013/176772A1 and WO/2013/142578A1, each of which is herein incorporated by reference.

In certain aspects, two separate Cas proteins (e.g., nickases) specific for a target site on each strand of dsDNA can create overhanging sequences complementary to overhanging sequences on another nucleic acid, or a separate region on the same nucleic acid. The overhanging ends created by contacting a nucleic acid with two nickases specific for target sites on both strands of dsDNA can be either 5′ or 3′ overhanging ends. For example, a first nickase can create a single strand break on the first strand of dsDNA, while a second nickase can create a single strand break on the second strand of dsDNA such that overhanging sequences are created. The target sites of each nickase creating the single strand break can be selected such that the overhanging end sequences created are complementary to overhanging end sequences on a different nucleic acid molecule. The complementary overhanging ends of the two different nucleic acid molecules can be annealed by the methods disclosed herein. In some aspects, the target site of the nickase on the first strand is different from the target site of the nickase on the second strand.

In some aspects, the first nucleic acid comprises a mutation that disrupts at least one amino acid residue of nuclease active sites in the Cas protein, wherein the mutant Cas protein generates a break in only one strand of the target DNA region, and wherein the mutation diminishes non-homologous recombination in the target DNA region. In one aspect, the first nucleic acid that encodes the Cas protein further comprises a nuclear localization signal (NLS). In one aspect, the nuclear localization signal is a SV40 nuclear localization signal.

### TALEN

In some aspects, the nuclease agent employed in the various methods and compositions disclosed herein can comprise a TALEN system. Thus, in one aspect, the nuclease agent is a Transcription Activator-Like Effector Nuclease (TALEN). TAL effector nucleases are a class of sequence-specific nucleases that can be used to make double-strand breaks at specific target sequences in the genome of a prokaryotic or eukaryotic organism. TAL effector nucleases are created by fusing a native or engineered transcription activator-like (TAL) effector, or functional part thereof, to the catalytic domain of an endonuclease, such as, for example, FokI.

The unique, modular TAL effector DNA binding domain allows for the design of proteins with potentially any given DNA recognition specificity. Thus, the DNA binding domains of the TAL effector nucleases can be engineered to recognize specific DNA target sites and thus, used to make double-strand breaks at desired target sequences. See, WO 2010/079430; Morbitzer et al. (2010) PNAS 10.1073/pnas. 1013133107; Scholze & Boch (2010) Virulence 1:428-432; Christian et al. Genetics (2010) 186:757-761; Li et al. (2010) Nuc. Acids Res. (2010) doi: 10.1093/nar/gkq704; and Miller et al. (2011) Nature Biotechnology 29:143-148; all of which are herein incorporated by reference.

Examples of suitable TAL nucleases, and methods for preparing suitable TAL nucleases, are disclosed, e.g., in US Patent Application No. 2011/0239315 A1, 2011/0269234 A1, 2011/0145940 A1, 2003/0232410 A1, 2005/0208489 A1, 2005/0026157 A1, 2005/0064474 A1, 2006/0188987 A1, and 2006/0063231 A1 (each hereby incorporated by reference).

In various aspects, TAL effector nucleases are engineered that cut in or near a target nucleic acid sequence in, e.g., a genomic locus of interest, wherein the target nucleic acid sequence is at or near a sequence to be modified by a targeting vector. The TAL nucleases suitable for use with the various methods and compositions provided herein include those that are specifically designed to bind at or near target nucleic acid sequences to be modified by targeting vectors as described herein.

In one aspect, each monomer of the TALEN comprises 12-25 TAL repeats, wherein each TAL repeat binds a 1 bp subsite. In one aspect, the nuclease agent is a chimeric protein comprising a TAL repeat-based DNA binding domain operably linked to an independent nuclease. In one aspect, the independent nuclease is a FokI endonuclease. In one aspect, the nuclease agent comprises a first TAL-repeat-based DNA binding domain and a second TAL-repeat-based DNA binding domain, wherein each of the first and the second TAL-repeat-based DNA binding domain is operably linked to a FokI nuclease, wherein the first and the second TAL-repeat-based DNA binding domain recognize two contiguous target DNA sequences in each strand of the target DNA sequence separated by about 6 bp to about 40 bp cleavage site, and wherein the FokI nucleases dimerize and make a double strand break at a target sequence.

In one aspect, the nuclease agent comprises a first TAL-repeat-based DNA binding domain and a second TAL-repeat-based DNA binding domain, wherein each of the first and the second TAL-repeat-based DNA binding domain is operably linked to a FokI nuclease, wherein the first and the second TAL-repeat-based DNA binding domain recognize two contiguous target DNA sequences in each strand of the target DNA sequence separated by a 5 bp or 6 bp cleavage site, and wherein the FokI nucleases dimerize and make a double strand break.

### Zinc-Finger Nuclease (ZFN)

In some aspects, the nuclease agent employed in the various methods and compositions disclosed herein can comprise a zinc-finger nuclease (ZFN) system. In one aspect, each monomer of the ZFN comprises 3 or more zinc finger-based DNA binding domains, wherein each zinc finger-based DNA binding domain binds to a 3 bp subsite. In other aspects, the ZEN is a chimeric protein comprising a zinc finger-based DNA binding domain operably linked to an independent nuclease. In one aspect, the independent endonuclease is a FokI endonuclease. In one aspect, the nuclease agent comprises a first ZFN and a second ZFN, wherein each of the first ZFN and the second ZFN is operably linked to a FokI nuclease, wherein the first and the second ZFN recognize two contiguous target DNA sequences in each strand of the target DNA sequence separated by about 6 bp to about 40 bp cleavage site or about a 5 bp to about 6 bp cleavage site, and wherein the FokI nucleases dimerize and make a double strand break. See, for example, US 20060246567; US 20080182332; US 20020081614; US 20030021776; WO/2002/057308A2; US 20130123484; US 20100291048; and, WO/2011/017293A2, each of which is herein incorporated by reference.

### Meganucleases

In some aspects, the nuclease agent employed in the various methods and compositions disclosed herein can comprise a meganuclease system. Meganucleases (or homing endonucleases or HEases) have been classified into four families based on conserved sequence motifs, the families are the “LAGLIDADG,” “GIY-YIG,” “H-N-H,” and “His-Cys box” families. These motifs participate in the coordination of metal ions and hydrolysis of phosphodiester bonds.

HEases are notable for their long recognition sites, and for tolerating some sequence polymorphisms in their DNA substrates. Meganuclease domains, structure and function are known, see for example, Guhan and Muniyappa (2003) Crit Rev Biochem Mol Biol 38:199-248; Lucas et al., (2001) Nucleic Acids Res 29:960-9; Jurica and Stoddard, (1999) Cell Mol Life Sci 55:1304-26; Stoddard, (2006) Q Rev Biophys 38:49-95; and Moure et al., (2002) Nat Struct Biol 9:764.

In some examples a naturally occurring variant, and/or engineered derivative meganuclease is used. Methods for modifying the kinetics, cofactor interactions, expression, optimal conditions, and/or recognition site specificity, and screening for activity are known, see for example, Epinat et al., (2003) Nucleic Acids Res 31:2952-62; Chevalier et al., (2002) Mol Cell 10:895-905; Gimble et al., (2003) Mol Biol 334:993-1008; Seligman et al., (2002) Nucleic Acids Res 30:3870-9; Sussman et al., (2004) J Mol Biol 342:31-41; Rosen et al., (2006) Nucleic Acids Res 34:4791-800; Chames et al., (2005) Nucleic Acids Res 33:e178; Smith et al., (2006) Nucleic Acids Res 34:e149; Gruen et al., (2002) Nucleic Acids Res 30:e29; Chen and Zhao, (2005) Nucleic Acids Res 33:e154; WO2005105989; WO2003078619; WO2006097854; WO2006097853; WO2006097784; and WO2004031346.

Any meganuclease can be used herein, including, but not limited to, I-SceI, I-SceII, I-SceIII, I-SceIV, I-SceV, I-SecVI, I-SceVII, I-CeuI, I-CeuAIIP, I-CreI, I-CrepsbIP, I-CrepsbIIP, I-CrepsbIIIP, I-CrepsbIVP, I-TliI, I-PpoI, PI-PspI, F-SceI, F-SceII, F-SuvI, F-TevI, F-TevII, I-AmaI, I-AniI, I-ChuI, I-CmoeI, I-CpaI, I-CpaII, I-CsmI, I-CvuI, I-CvuAIP, I-DdiI, I-DdiII, I-DirI, I-DmoI, I-HmuI, I-HmuII, I-HsNIP, I-LlaI, I-MsoI, I-NaaI, I-NanI, I-NcIIP, I-NgrIP, I-NitI, I-NjaI, I-Nsp236IP, I-PakI, I-PboIP, I-PcuIP, I-PcuAI, I-PcuVI, I-PgrIP, I-PobIP, I-PorIIP, I-PbpIP, I-SpBetaIP, I-ScaI, I-SexIP, I-SneIP, I-SpomI, I-SpomCP, I-SpomIP, I-SpomIIP, I-SquIP, I-Ssp6803I, I-SthPhiJP, I-SthPhiST3P, I-SthPhiSTe3bP, I-TdeIP, I-TevI, I-TevII, I-TevIII, I-UarAP, I-UarHGPAIP, I-UarHGPA13P, I-VinIP, I-ZbiIP, PI-MtuI, PI-MtuHIP, PI-MtuHIIP, PI-PfuI, PI-PfuII, PI-PkoI, PI-PkoII, PI-Rma43812IP, PI-SpBetaIP, PI-SceI, PI-TfuI, PI-TfuII, PI-ThyI, PI-TliI, PI-TliII, or any active variants or fragments thereof.

In one aspect, the meganuclease recognizes double-stranded DNA sequences of 12 to 40 base pairs. In one aspect, the meganuclease recognizes one perfectly matched target sequence in one of the heterologous plasmids described herein. In one aspect, the meganuclease is a homing nuclease. In one aspect, the homing nuclease is a “LAGLIDADG” family of homing nuclease. In one aspect, the “LAGLIDADG” family of homing nuclease is selected from I-SceI, I-CreI, and I-DmoI.

### Restriction Endonucleases

In some aspects, the nuclease agent employed for homologous recombination in the various methods and compositions disclosed herein can comprise a restriction endonuclease, which includes Type I, Type II, Type III, and Type IV endonucleases. Type I and Type III restriction endonucleases recognize specific recognition sites, but typically cleave at a variable position from the nuclease-binding site, which can be hundreds of base pairs away from the cleavage site (recognition site). In Type II systems the restriction activity is independent of any methylase activity, and cleavage typically occurs at specific sites within or near to the binding site. Most Type II enzymes cut palindromic sequences, however Type IIa enzymes recognize non-palindromic recognition sites and cleave outside of the recognition site, Type IIb enzymes cut sequences twice with both sites outside of the recognition site, and Type IIs enzymes recognize an asymmetric recognition site and cleave on one side and at a defined distance of about 1-20 nucleotides from the recognition site. Type IV restriction enzymes target methylated DNA. Restriction enzymes are further described and classified, for example in the REBASE database (webpage at rebase.neb.com; Roberts et al., (2003) Nucleic Acids Res 31:418-20), Roberts et al., (2003) Nucleic Acids Res 31:1805-12, and Belfort et al., (2002) in Mobile DNA II, pp. 761-783, Eds. Craigie et al., (ASM Press, Washington, D.C.).

### X. Kits and Articles of Manufacture

The disclosure also provides kits and products of manufacture comprising, for example,
(i) a bicistronic polynucleotide of the present disclosure;
(ii) a vector comprising a bicistronic polynucleotide of the present disclosure;
(iii) a cell comprising a bicistronic polynucleotide of the present disclosure; and optionally,
(iv) instructions for use.

In some aspects, the disclosure provides kits and products of manufacture comprising, for example, a cell genetically modified to express, e.g., a CAR of the present disclosure, i.e., a cell comprising one or more polynucleotides encoding, e.g., a CAR of the present disclosure, or one or more vectors encoding, e.g., a CAR of the present disclosure (e.g., a T cell, a natural killer (NK) cell, an natural killer T (NKT) cell, or an ILC cell), or a pharmaceutical composition comprising the cell, and optionally instructions for use.

In some aspects, the disclosure provides kits and products of manufacture comprising an oligonucleotide for nuclease-mediated insertion in the B2M gene selected from Site 1 (ACTCTCTCTTTCTGGCCTGG; SEQ ID NO: 1); Site 2 (AGTCACATGGTTCACACGGC; SEQ ID NO: 2); Site 3 (CACAGCCCAAGATAGTTAAG; SEQ ID NO: 3); and Site 4 (GAGACATGTAAGCAGCATCA; SEQ ID NO: 4).

In some aspects, the disclosure provides kits and products of manufacture comprising an oligonucleotide for nuclease-mediated insertion in the B2M gene, wherein the oligonucleotide hybridized under stringent conditions with an oligonucleotide selected from Site 1 (ACTCTCTCTTTCTGGCCTGG; SEQ ID NO: 1); Site 2 (AGTCACATGGTTCACACGGC; SEQ ID NO: 2); Site 3 (CACAGCCCAAGATAGTTAAG; SEQ ID NO: 3); and Site 4 (GAGACATGTAAGCAGCATCA; SEQ ID NO: 4), and any of their complementary sequences.

In some aspects, the disclosure provides kits and products of manufacture comprising a gRNA for CRISPR/Cas9 mediated insertion in the B2M, wherein the gRNA is selected from Site 1 gRNA (ACTCTCTCTTTCTGGCCTGG; SEQ ID NO: 1); Site 2 gRNA (AGTCACATGGTTCACACGGC; SEQ ID NO: 2); Site 3 gRNA (CACAGCCCAAGATAGTTAAG; SEQ ID NO: 3); and Site 4 gRNA (GAGACATGTAAGCAGCATCA; SEQ ID NO: 4), and any of their complementary sequences.

In some aspects, the kit or product of manufacture comprises at least a bicistronic polynucleotide encoding, e.g., a CAR of the present disclosure, a vector comprising a bicistronic polynucleotide encoding, e.g., a CAR of the present disclosure, a cell genetically modified to express, e.g., a CAR encoded by a bicistronic polynucleotide of the present disclosure, a composition (e.g., a pharmaceutical composition comprising a bicistronic polynucleotide, vector, or cell disclosed herein), or an oligonucleotide for nuclease-mediated insertion, in one or more containers.

In some aspects, the kit or product of manufacture comprises at least a bicistronic polynucleotide encoding, e.g., a CAR of the present disclosure, a vector comprising a bicistronic polynucleotide encoding, e.g., a CAR of the present disclosure, a cell genetically modified to express, e.g., a CAR encoded by a bicistronic polynucleotide of the present disclosure, a composition (e.g., a pharmaceutical composition comprising a bicistronic polynucleotide, vector, or cell disclosed herein), or an oligonucleotide for nuclease-mediated insertion, and optionally a brochure.

In some aspects, the kit or product of manufacture comprises at least a bicistronic polynucleotide encoding, e.g., a CAR of the present disclosure, a vector comprising a bicistronic polynucleotide encoding, e.g., a CAR of the present disclosure, a cell genetically modified to express, e.g., a CAR encoded by a bicistronic polynucleotide of the present disclosure, a composition (e.g., a pharmaceutical composition comprising a bicistronic polynucleotide, vector, or cell disclosed herein), or an oligonucleotide for nuclease-mediated insertion, and optionally at least a vial with a solvent or reagent.

In some aspects, the kit or product of manufacture comprises, e.g., a bicistronic polynucleotide of the present disclosure, or a vector comprising a bicistronic polypeptide of the present disclosure, in at least one container, and another or more containers with transfection reagents.

In some aspects, the kit or product of manufacture comprises a gRNA oligonucleotide selected from Site 1 (ACTCTCTCTTTCTGGCCTGG; SEQ ID NO: 1); Site 2 (AGTCACATGGTTCACACGGC; SEQ ID NO:2); Site 3 (CACAGCCCAAGATAGTTAAG; SEQ ID NO:3); and Site 4 (GAGACATGTAAGCAGCATCA; SEQ ID NO: 4) and their respective complementary sequences in at least one container, and another or more containers for nuclease-mediated insertion (e.g., CRISPR-Cas reagents).

One skilled in the art will readily recognize that the bicistronic polynucleotide encoding a CAR of the present disclosure, vector comprising a bicistronic polynucleotide (e.g., encoding a CAR) of the present disclosure, cell genetically modified to express, e.g., a CAR encoded by a bicistronic polynucleotide of the present disclosure, composition (e.g., a pharmaceutical composition comprising a bicistronic polynucleotide, vector, or cell disclosed herein), or oligonucleotide for nuclease-mediated insertion, or combinations thereof can be readily incorporated into one of the established kit formats which are well known in the art.

SEQUENCES
Sequence information:
anti-GD2 CAR
ATGGAACTGGGACTGTCATGGATCTTCTTGCTGGCTATCCTGAAGGGAGTGCAGTGTGAAGTTCAGCTGCTGCAG
AGCGGACCCGAACTGGAAAAACCTGGCGCCTCCGTGATGATCAGCTGCAAGGCCTCTGGCAGCTCCTTCACCGGC
TACAACATGAACTGGGTCCGACAGAACATCGGCAAGAGCCTGGAATGGATCGGCGCCATCGATCCTTACTACGGC
GGCACCAGCTACAACCAGAAGTTCAAGGGCAGAGCCACACTGACCGTGGACAAGAGCAGCAGCACAGCCTACATG
CATCTGAAGTCCCTGACCAGCGAGGACAGCGCCGTGTACTACTGTGTGTCCGGCATGGAATACTGGGGCCAGGGC
ACAAGCGTGACAGTCTCTTCTGGCGGCGGTGGATCTGGCGGAGGCGGAAGTGGTGGCGGCGGATCTGATGTGGTC
ATGACACAGACCCCTCTGAGCCTGCCTGTGTCTCTGGGAGATCAGGCCAGCATCAGCTGTAGAAGCAGCCAGAGC
CTGGTGCACAGAAACGGCAACACCTACCTGCACTGGTATCTGCAGAAGCCCGGCCAGTCTCCTAAGCTGCTGATC
CACAAGGTGTCCAACAGATTCAGCGGCGTGCCCGACAGATTCTCTGGCTCTGGAAGCGGCACCGACTTCACCCTG
AAGATTAGCAGAGTGGAAGCCGAGGACCTGGGCGTGTACTTCTGTAGCCAGAGCACACACGTGCCACCTCTGACA
TTTGGCGCTGGCACCAAGCTGGAACTGAAAAGGACCACGACGCCAGCGCCGCGACCACCAACACCGGCGCCCACC
ATCGCGTCGCAGCCCCTGTCCCTGCGCCCAGAGGCGTGCCGGCCAGCGGCGGGGGGCGCAGTGCACACGAGGGGG
CTGGACTTCGCCTGTGATTTTTGGGTGCTGGTGGTGGTTGGTGGAGTCCTGGCTTGCTATAGCTTGCTAGTAACA
GTGGCCTTTATTATTTTCTGGGTGAGGAGTAAGAGGAGCAGGCTCCTGCACAGTGACTACATGAACATGACTCCC
CGCCGCCCCGGGCCCACCCGCAAGCATTACCAGCCCTATGCCCCACCACGCGACTTCGCAGCCTATCGCTCCAAA
CGGGGCAGAAAGAAACTCCTGTATATATTCAAACAACCATTTATGAGACCAGTACAAACTACTCAAGAGGAAGAT
GGCTGTAGCTGCCGATTTCCAGAAGAAGAAGAAGGAGGATGTGAACTGAGAGTGAAGTTCAGCAGGAGCGCAGAC
GCCCCCGCGTACCAGCAGGGCCAGAACCAGCTCTATAACGAGCTCAATCTAGGACGAAGAGAGGAGTACGATGTT
TTGGACAAGAGACGTGGCCGGGACCCTGAGATGGGGGGAAAGCCGAGAAGGAAGAACCCTCAGGAAGGCCTGTAC
AATGAACTGCAGAAAGATAAGATGGCGGAGGCCTACAGTGAGATTGGGATGAAAGGCGAGCGCCGGAGGGGCAAG
GGGCACGATGGCCTTTACCAGGGTCTCAGTACAGCCACCAAGGACACCTACGACGCCCTTCACATGCAGGCCCTG
CCCCCTCGC
(SEQ ID NO: 19)
MELGLSWIFLLAILKGVQCEVQLLQSGPELEKPGASVMISCKASGSSFTGYNMNWVRQNIGKSLEWIGAIDPYYG
GTSYNQKFKGRATLTVDKSSSTAYMHLKSLTSEDSAVYYCVSGMEYWGQGTSVTVSSGGGGSGGGGSGGGGSDVV
MTQTPLSLPVSLGDQASISCRSSQSLVHRNGNTYLHWYLQKPGQSPKLLIHKVSNRFSGVPDRFSGSGSGTDFTL
KISRVEAEDLGVYFCSQSTHVPPLTFGAGTKLELKRTTTPAPRPPTPAPTIASQPLSLRPEACRPAAGGAVHTRG
LDFACDFWVLVVVGGVLACYSLLVTVAFIIFWVRSKRSRLLHSDYMNMTPRRPGPTRKHYQPYAPPRDFAAYRSK
RGRKKLLYIFKQPFMRPVQTTQEEDGCSCRFPEEEEGGCELRVKFSRSADAPAYQQGQNQLYNELNLGRREEYDV
LDKRRGRDPEMGGKPRRKNPQEGLYNELQKDKMAEAYSEIGMKGERRRGKGHDGLYQGLSTATKDTYDALHMQAL
PPR (SEQ ID NO: 20)

| | | SEQ | |
|---|---|---|---|
| | | ID | |
| Description | Type | NO | Sequence |
| Signal | Nucleic | 21 | ATGGAACTGGGACTGTCATGGATCTTCTTGCTGGCTATCCTGAAGGGAGTG |
| peptide | acid | | CAGTGT |
| | Protein | 22 | MELGLSWIFLLAILKGVQC |
| anti- | Nucleic | 23 | GAAGTTCAGCTGCTGCAGAGCGGACCCGAACTGGAAAAACCTGGCGCCTCC |
| GD2scFv | acid | | GTGATGATCAGCTGCAAGGCCTCTGGCAGCTCCTTCACCGGCTACAACATG |
| | | | AACTGGGTCCGACAGAACATCGGCAAGAGCCTGGAATGGATCGGCGCCATC |
| | | | GATCCTTACTACGGCGGCACCAGCTACAACCAGAAGTTCAAGGGCAGAGCC |
| | | | ACACTGACCGTGGACAAGAGCAGCAGCACAGCCTACATGCATCTGAAGTCC |
| | | | CTGACCAGCGAGGACAGCGCCGTGTACTACTGTGTGTCCGGCATGGAATAC |
| | | | TGGGGCCAGGGCACAAGCGTGACAGTCTCTTCTGGCGGCGGTGGATCTGGC |
| | | | GGAGGCGGAAGTGGTGGCGGCGGATCTGATGTGGTCATGACACAGACCCCT |
| | | | CTGAGCCTGCCTGTGTCTCTGGGAGATCAGGCCAGCATCAGCTGTAGAAGC |
| | | | AGCCAGAGCCTGGTGCACAGAAACGGCAACACCTACCTGCACTGGTATCTG |
| | | | CAGAAGCCCGGCCAGTCTCCTAAGCTGCTGATCCACAAGGTGTCCAACAGA |
| | | | TTCAGCGGCGTGCCCGACAGATTCTCTGGCTCTGGAAGCGGCACCGACTTC |
| | | | ACCCTGAAGATTAGCAGAGTGGAAGCCGAGGACCTGGGCGTGTACTTCTGT |
| | | | AGCCAGAGCACACACGTGCCACCTCTGACATTTGGCGCTGGCACCAAGCTG |
| | | | GAACTGAAAAGG |
| | Protein | 24 | EVQLLQSGPELEKPGASVMISCKASGSSFTGYNMNWVRQNIGKSLEWIGAI |
| | | | DPYYGGTSYNQKFKGRATLTVDKSSSTAYMHLKSLTSEDSAVYYCVSGMEY |
| | | | WGQGTSVTVSSGGGGSGGGGSGGGGSDVVMTQTPLSLPVSLGDQASISCRS |
| | | | SQSLVHRNGNTYLHWYLQKPGQSPKLLIHKVSNRFSGVPDRFSGSGSGTDF |
| | | | TLKISRVEAEDLGVYFCSQSTHVPPLTFGAGTKLELKR |
| CD8alpha | Nucleic | 25 | ACCACGACGCCAGCGCCGCGACCACCAACACCGGCGCCCACCATCGCGTCG |
| hinge | acid | | CAGCCCCTGTCCCTGCGCCCAGAGGCGTGCCGGCCAGCGGCGGGGGGCGCA |
| | | | GTGCACACGAGGGGGCTGGACTTCGCCTGTGAT |
| | Protein | 26 | TTTPAPRPPTPAPTIASQPLSLRPEACRPAAGGAVHTRGLDFACD |
| CD28 TM/ | Nucleic | 27 | TTTTGGGTGCTGGTGGTGGTTGGTGGAGTCCTGGCTTGCTATAGCTTGCTA |
| IC domains | acid | | GTAACAGTGGCCTTTATTATTTTCTGGGTGAGGAGTAAGAGGAGCAGGCTC |
| | | | CTGCACAGTGACTACATGAACATGACTCCCCGCCGCCCCGGGCCCACCCGC |
| | | | AAGCATTACCAGCCCTATGCCCCACCACGCGACTTCGCAGCCTATCGCTCC |
| | Protein | 28 | FWVLVVVGGVLACYSLLVTVAFIIFWVRSKRSRLLHSDYMNMTPRRPGPTR |
| | | | KHYQPYAPPRDFAAYRS |
| 4-1BB | Nucleic | 29 | AAACGGGGCAGAAAGAAACTCCTGTATATATTCAAACAACCATTTATGAGA |
| activation | acid | | CCAGTACAAACTACTCAAGAGGAAGATGGCTGTAGCTGCCGATTTCCAGAA |
| domain | | | GAAGAAGAAGGAGGATGTGAACTG |
| | Protein | 30 | KRGRKKLLYIFKQPFMRPVQTTQEEDGCSCRFPEEEEGGCEL |
| CD3zeta | Nucleic | 31 | AGAGTGAAGTTCAGCAGGAGCGCAGACGCCCCCGCGTACCAGCAGGGCCAG |
| activation | acid | | AACCAGCTCTATAACGAGCTCAATCTAGGACGAAGAGAGGAGTACGATGTT |
| domain | | | TTGGACAAGAGACGTGGCCGGGACCCTGAGATGGGGGGAAAGCCGAGAAGG |
| | | | AAGAACCCTCAGGAAGGCCTGTACAATGAACTGCAGAAAGATAAGATGGCG |
| | | | GAGGCCTACAGTGAGATTGGGATGAAAGGCGAGCGCCGGAGGGGCAAGGGG |
| | | | CACGATGGCCTTTACCAGGGTCTCAGTACAGCCACCAAGGACACCTACGAC |
| | | | GCCCTTCACATGCAGGCCCTGCCCCCTCGC |
| | Protein | 32 | RVKFSRSADAPAYQQGQNQLYNELNLGRREEYDVLDKRRGRDPEMGGKPRR |
| | | | KNPQEGLYNELQKDKMAEAYSEIGMKGERRRGKGHDGLYQGLSTATKDTYD |
| | | | ALHMQALPPR |

HLA-E dimer
ATCCAGCGTACTCCAAAGATTCAGGTTTACTCACGTCATCCAGCAGAGAATGGAAAGTCAAATTTCCTGAATTGCTATG
TGTCTGGGTTTCATCCATCCGACATTGAAGTTGACTTACTGAAGAATGGAGAGAGAATTGAAAAAGTGGAGCATTCAGA
CTTGTCTTTCAGCAAGGACTGGTCTTTCTATCTCTTGTACTACACTGAATTCACCCCCACTGAAAAAGATGAGTATGCC
TGCCGTGTGAACCATGTGACTTTGTCACAGCCCAAGATAGTTAAGTGGGATCGAGACATGGGTGGAGGCGGTTCTGGAG
GTGGCGGAAGTGGCGGCGGGGGAAGCGGTGGGGGGGGTTCCGGCTCCCACTCCTTGAAGTATTTCCACACTTCCGTGTC
CCGGCCCGGCCGCGGGGAGCCCCGCTTCATCTCTGTGGGCTACGTGGACGACACCCAGTTCGTGCGCTTCGACAACGAC
GCCGCGAGTCCGAGGATGGTGCCGCGGGCGCCGTGGATGGAGCAGGAGGGGTCAGAGTATTGGGACCGGGAGACACGGA
GCGCCAGGGACACCGCACAGATTTTCCGAGTGAATCTGCGGACGCTGCGCGGCTACTACAATCAGAGCGAGGCCGGGTC
TCACACCCTGCAGTGGATGCATGGCTGCGAGCTGGGGCCCGACGGGCGCTTCCTCCGCGGGTATGAACAGTTCGCCTAC
GACGGCAAGGATTATCTCACCCTGAATGAGGACCTGCGCTCCTGGACCGCGGTGGACACGGCGGCTCAGATCTCCGAGC
AAAAGTCAAATGATGCCTCTGAGGCGGAGCACCAGAGAGCCTACCTGGAAGACACATGCGTGGAGTGGCTCCACAAATA
CCTGGAGAAGGGGAAGGAGACGCTGCTTCACCTGGAGCCCCCAAAGACACACGTGACTCACCACCCCATCTCTGACCAT
GAGGCCACCCTGAGGTGCTGGGCCCTGGGCTTCTACCCTGCGGAGATCACACTGACCTGGCAGCAGGATGGGGAGGGCC
ATACCCAGGACACGGAGCTCGTGGAGACCAGGCCTGCAGGGGATGGAACCTTCCAGAAGTGGGCAGCTGTGGTGGTGCC
TTCTGGAGAGGAGCAGAGATACACGTGCCATGTGCAGCATGAGGGGCTACCCGAGCCCGTCACCCTGAGATGGAAGCCG
GCTTCCCAGCCCACCATCCCCATCGTGGGCATCATTGCTGGCCTGGTTCTCCTTGGATCTGTGGTCTCTGGAGCTGTGG
TTGCTGCTGTGATATGGAGGAAGAAGAGCTCAGGTGGAAAAGGAGGGAGCTACTCTAAGGCTGAGTGGAGCGACAGTGC
CCAGGGGTCTGAGTCTCACAGCTTGTAA
(SEQ ID NO: 33)
IQRTPKIQVYSRHPAENGKSNFLNCYVSGFHPSDIEVDLLKNGERIEKVEHSDLSFSKDWSFYLLYYTEFTPTEKDEYA
CRVNHVTLSQPKIVKWDRDMGGGGSGGGGSGGGGSGGGGSGSHSLKYFHTSVSRPGRGEPRFISVGYVDDTQFVRFDND
AASPRMVPRAPWMEQEGSEYWDRETRSARDTAQIFRVNLRTLRGYYNQSEAGSHTLQWMHGCELGPDGRFLRGYEQFAY
DGKDYLTLNEDLRSWTAVDTAAQISEQKSNDASEAEHQRAYLEDTCVEWLHKYLEKGKETLLHLEPPKTHVTHHPISDH
EATLRCWALGFYPAEITLTWQQDGEGHTQDTELVETRPAGDGTFQKWAAVVVPSGEEQRYTCHVQHEGLPEPVTLRWKP
ASQPTIPIVGIIAGLVLLGSVVSGAVVAAVIWRKKSSGGKGGSYSKAEWSDSAQGSESHSL
(SEQ ID NO: 34)

| | | SEQ | |
|---|---|---|---|
| | | ID | |
| Description | Type | NO | Sequence |
| Beta2- | Nucleic | 35 | ATGTCTCGCTCCGTGGCCTTAGCTGTGCTCGCGCTACTCTCTCTTTCT |
| microglobulin | acid | | GGGCTAGAAGCTATCCAGCGTACTCCAAAGATTCAGGTTTACTCACGT |
| | | | CATCCAGCAGAGAATGGAAAGTCAAATTTCCTGAATTGCTATGTGTCT |
| | | | GGGTTTCATCCATCCGACATTGAAGTTGACTTACTGAAGAATGGAGAG |
| | | | AGAATTGAAAAAGTGGAGCATTCAGACTTGTCTTTCAGCAAGGACTGG |
| | | | TCTTTCTATCTCTTGTACTACACTGAATTCACCCCCACTGAAAAAGAT |
| | | | GAGTATGCCTGCCGTGTGAACCATGTGACTTTGTCACAGCCCAAGATA |
| | | | GTTAAGTGGGATCGAGACATG |
| | Protein | 36 | MSRSVALAVLALLSLSGLEAIQRTPKIQVYSRHPAENGKSNFLNCYVS |
| | | | GFHPSDIEVDLLKNGERIEKVEHSDLSFSKDWSFYLLYYTEFTPTEKD |
| | | | EYACRVNHVTLSQPKIVKWDRDM |
| 4X G4S linker | Nucleic | 37 | GGTGGAGGCGGTTCTGGAGGTGGCGGAAGTGGCGGCGGGGGAAGCGGT |
| | acid | | GGGGGCGGTTCC |
| | Protein | 38 | GGGGSGGGGSGGGGSGGGGS |
| Mature HLA-E | Nucleic | 39 | GGCTCCCACTCCTTGAAGTATTTCCACACTTCCGTGTCCCGGCCCGGC |
| | acid | | CGCGGGGAGCCCCGCTTCATCTCTGTGGGCTACGTGGACGACACCCAG |
| | | | TTCGTGCGCTTCGACAACGACGCCGCGAGTCCGAGGATGGTGCCGCGG |
| | | | GCGCCGTGGATGGAGCAGGAGGGGTCAGAGTATTGGGACCGGGAGACA |
| | | | CGGAGCGCCAGGGACACCGCACAGATTTTCCGAGTGAATCTGCGGACG |
| | | | CTGCGCGGCTACTACAATCAGAGCGAGGCCGGGTCTCACACCCTGCAG |
| | | | TGGATGCATGGCTGCGAGCTGGGGCCCGACGGGCGCTTCCTCCGCGGG |
| | | | TATGAACAGTTCGCCTACGACGGCAAGGATTATCTCACCCTGAATGAG |
| | | | GACCTGCGCTCCTGGACCGCGGTGGACACGGCGGCTCAGATCTCCGAG |
| | | | CAAAAGTCAAATGATGCCTCTGAGGCGGAGCACCAGAGAGCCTACCTG |
| | | | GAAGACACATGCGTG |
| | | | GAGTGGCTCCACAAATACCTGGAGAAGGGGAAGGAGACGCTGCTTCAC |
| | | | CTGGAGCCCCCAAAGACACACGTGACTCACCACCCCATCTCTGACCAT |
| | | | GAGGCCACCCTGAGGTGCTGGGCCCTGGGCTTCTACCCTGCGGAGATC |
| | | | ACACTGACCTGGCAGCAGGATGGGGAGGGCCATACCCAGGACACGGAG |
| | | | CTCGTGGAGACCAGGCCTGCAGGGGATGGAACCTTCCAGAAGTGGGCA |
| | | | GCTGTGGTGGTGCCTTCTGGAGAGGAGCAGAGATACACGTGCCATGTG |
| | | | CAGCATGAGGGGCTACCCGAGCCCGTCACCCTGAGATGGAAGCCGGCT |
| | | | TCCCAGCCCACCATCCCCATCGTGGGCATCATTGCTGGCCTGGTTCTC |
| | | | CTTGGATCTGTGGTCTCTGGAGCTGTGGTTGCTGCTGTGATATGGAGG |
| | | | AAGAAGAGCTCAGGTGGAAAAGGAGGGAGCTACTCTAAGGCTGAGTGG |
| | | | AGCGACAGTGCCCAGGGGTCTGAGTCTCACAGCTTGTAA |
| | Protein | 40 | GSHSLKYFHTSVSRPGRGEPRFISVGYVDDTQFVRFDNDAASPRMVPR |
| | | | APWMEQEGSEYWDRETRSARDTAQIFRVNLRTLRGYYNQSEAGSHTLQ |
| | | | WMHGCELGPDGRFLRGYEQFAYDGKDYLTLNEDLRSWTAVDTAAQISE |
| | | | QKSNDASEAEHQRAYLEDTCVEWLHKYLEKGKETLLHLEPPKTHVTHH |
| | | | PISDHEATLRCWALGFYPAEITLTWQQDGEGHTQDTELVETRPAGDGT |
| | | | FQKWAAVVVPSGEEQRYTCHVQHEGLPEPVTLRWKPASQPTIPIVGII |
| | | | AGLVLLGSVVSGAVVAAVIWRKKSSGGKGGSYSKAEWSDSAQGSESHS |
| | | | L |
| | Dinutuximab | | |
| | Chimeric IgG1-Kappa | | |
| | Heavy Chain | | |
| | (HC) VH (1-113) [Domain 1] + CH1 (114-211) [Domain 2] + CH2 (227-336) | | |
| | [Domain 3] + CH3 (337-441) [Domain 4] | | |
| | EVQLLQSGPELEKPGASVMISCKASGSSFTGYNMNWVRQNIGKSLEWIGAIDPYYGGTSYNQKFKGR | | |
| | ATLTVDKSSSTAYMHLKSLTSEDSAVYYCVSGMEYWGQGTSVTVSSASTKGPSVFPLAPSSKSTSGG | | |
| | TAALGCLVKDYFPEPVTVSWNSGALTSGVHTFPAVLQSSGLYSLSSVVTVPSSSLGTQTYICNVNHK | | |
| | PSNTKVDKRVEPKSCDKTHTCPPCPAPELLGGPSVFLFPPKPKDTLMISRTPEVTCVVVDVSHEDPE | | |
| | VKFNWYVDGVEVHNAKTKPREEQYNSTYRVVSVLTVLHQDWLNGKEYKCKVSNKALPAPIEKTISKA | | |
| | KGQPREPQVYTLPPSREEMTKNQVSLTCLVKGFYPSDIAVEWESNGQPENNYKTTPPVLDSDGSFFL | | |
| | YSKLTVDKSRWQQGNVFSCSVMHEALHNHYTQKSLSLSPGK | | |
| | (SEQ ID NO: 41) | | |
| | Light Chain (LC) | | |
| | V-KAPPA (1-113) [Domain 1] + C-KAPPA [Domain 2] | | |
| | EIVMTQSPATLSVSPGERATLSCRSSQSLVHRNGNTYLHWYLQKPGQSPKLLIHKVSNRFSGVPDRF | | |
| | SGSGSGTDFTLKISRVEAEDLGVYFCSQSTHVPPLTFGAGTKLELKRTVAAPSVFIFPPSDEQLKSG | | |
| | TASVVCLLNNFYPREAKVQWKVDNALQSGNSQESVTEQDSKDSTYSLSSTLTLSKADYEKHKVYACE | | |
| | VTHQGLSSPVTKSFNRGEC | | |
| | (SEQ ID NO: 42) | | |
| | | SEQ | |
| | | ID | |
| Description | Type | NO | Sequence |
| VH | Nucleic | 43 | GAAGTTCAGCTGCTGCAGAGCGGACCCGAACTGGAAAAACCTGGCGCCTCCG |
| | acid | | TGATGATCAGCTGCAAGGCCTCTGGCAGCTCCTTCACCGGCTACAACATGAA |
| | | | CTGGGTCCGACAGAACATCGGCAAGAGCCTGGAATGGATCGGCGCCATCGAT |
| | | | CCTTACTACGGCGGCACCAGCTACAACCAGAAGTTCAAGGGCAGAGCCACAC |
| | | | TGACCGTGGACAAGAGCAGCAGCACAGCCTACATGCATCTGAAGTCCCTGAC |
| | | | CAGCGAGGACAGCGCCGTGTACTACTGTGTGTCCGGCATGGAATACTGGGGC |
| | | | CAGGGCACAAGCGTGACAGTCTCTTCT |
| | Protein | 44 | EVQLLQSGPELEKPGASVMISCKASGSSFTGYNMNWVRQNIGKSLEWIGAID |
| | | | PYYGGTSYNQKFKGRATLTVDKSSSTAYMHLKSLTSEDSAVYYCVSGMEYWG |
| | | | QGTSVTVSS |
| VL | Nucleic | 45 | GAAATCGTAATGACTCAGAGCCCGGCTACCCTTTCCGTCTCACCGGGAGAGA |
| | acid | | GAGCAACCCTTAGCTGTAGAAGCAGCCAGAGCCTGGTGCACAGAAACGGCAA |
| | | | CACCTACCTGCACTGGTATCTGCAGAAGCCCGGCCAGTCTCCTAAGCTGCTG |
| | | | ATCCACAAGGTGTCCAACAGATTCAGCGGCGTGCCCGACAGATTCTCTGGCT |
| | | | CTGGAAGCGGCACCGACTTCACCCTGAAGATTAGCAGAGTGGAAGCCGAGGA |
| | | | CCTGGGCGTGTACTTCTGTAGCCAGAGCACACACGTGCCACCTCTGACATTT |
| | | | GGCGCTGGCACCAAGCTGGAACTGAAA |
| | Protein | 46 | EIVMTQSPATLSVSPGERATLSCRSSQSLVHRNGNTYLHWYLQKPGQSPKLL |
| | | | IHKVSNRFSGVPDRFSGSGSGTDFTLKISRVEAEDLGVYFCSQSTHVPPLTF |
| | | | GAGTKLELK |
| VH CDR1 | Protein | 47 | GSSFTGYN |
| VH CDR2 | Protein | 48 | IDPYYGGT |
| VH CDR3 | Protein | 49 | VSGMEY |
| VL CDR1 | Protein | 50 | QSLVHRNGNTY |
| VL CDR2 | Protein | 51 | KVS |
| VL CDR3 | Protein | 52 | SQSTHVPPLT |

*CDR numbering is IMGT

| | | SEQ | | | | | |
|---|---|---|---|---|---|---|---|
| | | ID | | | | ID | |
| | | NO | Sequence | | | NO | Sequence |
| VH | Chothia | 59 | GSSFTGY | VL | Chothia | 71 | RSSQSLVHRNGNTYLH |
| CDR1 | AbM | 60 | GSSFTGYNMN | CDR1 | AbM | 72 | RSSQSLVHRNGNTYLH |
| | Kabat | 61 | GYNMN | | Kabat | 73 | RSSQSLVHRNGNTYLH |
| | Contact | 62 | TGYNMN | | Contact | 74 | VHRNGNTYLHWY |
| | IMGT | 53 | GSSFTGYN | | IMGT | 56 | QSLVHRNGNTY |
| VH | Chothia | 63 | DPYYGG | VL | Chothia | 75 | KVSNRFS |
| CDR2 | AbM | 64 | AIDPYYGGTS | CDR2 | AbM | 76 | KVSNRFS |
| | Kabat | 65 | AIDPYYGGTSYNQKFKG | | Kabat | 77 | KVSNRFS |
| | Contact | 66 | WIGAIDPYYGGTS | | Contact | 78 | LLIHKVSNRF |
| | IMGT | 54 | IDPYYGGT | | IMGT | 57 | KV |
| VH | Chothia | 67 | GMEY | VL | Chothia | 79 | SQSTHVPPLT |
| CDR3 | AbM | 68 | GMEY | CDR3 | AbM | 80 | SQSTHVPPLT |
| | Kabat | 69 | GMEY | | Kabat | 81 | SQSTHVPPLT |
| | Contact | 70 | VSGME | | Contact | 82 | SQSTHVPPL |
| | IMGT | 55 | VSGMEY | | IMGT | 58 | SQSTHVPPLT |

CDR locations identified using Abysis Key Annotation version 3.4.1 available at abysis dot org

### EXAMPLES

### Example 1

### CRISPR-Mediated Disruption of B2M Decreased Cell Surface Expression of B2M and HLA-A/B/C

Immune rejection of heterologous CAR-T cells is mostly due to HLA-I presenting the donor's cells as “non-self” to the host immune system. HLA class I molecules (including HLA-A/B/C) on activated T cells are expressed as heterodimers, which include the beta 2-microglobulin (B2M) subunit. Elimination of B2M, which is required for HLA expression on a cell surface, via nucleases such as CRISPR/Cas removes the ability of the cell to be recognized as non-self by HLA mismatch.

Four CRISPR reagents, guide RNAs or gRNAs, directed to four insertion sites in the B2M gene designated Site1 (ACTCTCTCTTTCTGGCCTGG; SEQ ID NO: 1), Site2 (AGTCACATGGTTCACACGGC; SEQ ID NO: 2), Site 3 (CACAGCCCAAGATAGTTAAG; SEQ ID NO: 3), Site 4 (GAGACATGTAAGCAGCATCA; SEQ ID NO: 4)) were designed to specifically target the Cas9 nuclease activity to those insertion sites.

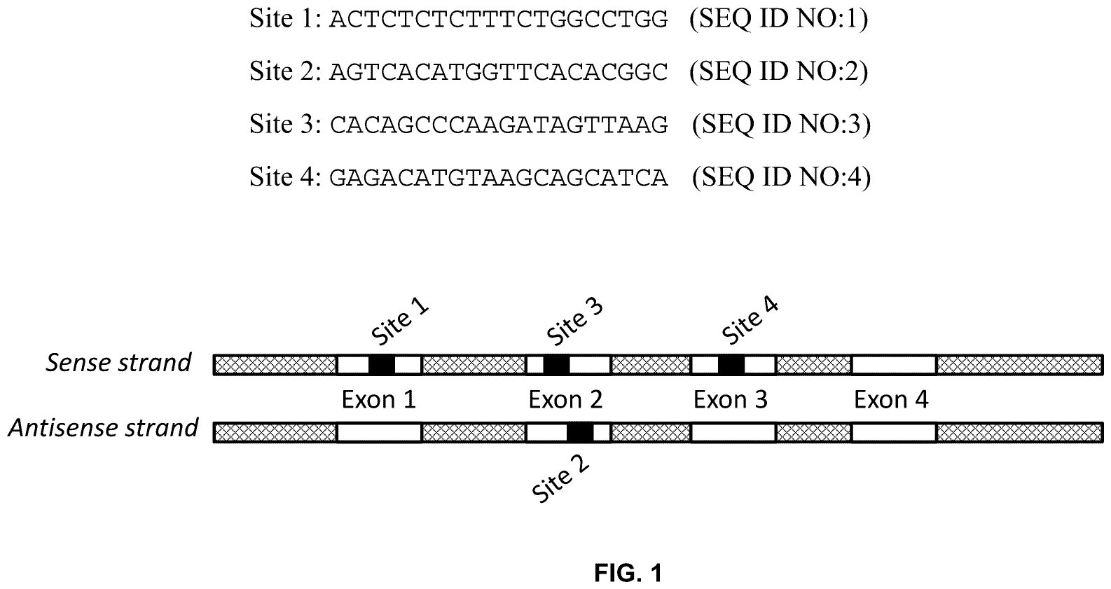

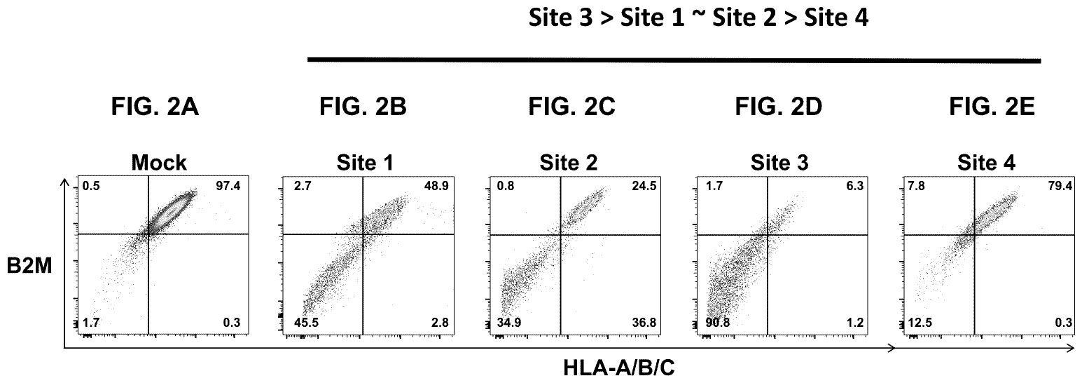

All four insertion sites were located within exons of the B2M locus of CD4+ T-cells. Sites 1, 3 and 4 were on the sense strand, whereas Site 2 was on the antisense strand (FIG. 1). Flow cytometry analysis showed that the disruption of the B2M locus with CRISPR reagents decreased the expression levels of B2M and HLA-A/B/C from the cell surface (FIGS. 2A-E). While all disruptions decreased B2M expression on the cell surface, the effect observed was site-dependent. In order of progressive B2M disruption, the four sites disclosed herein can be ranked as follows: Site 3>Site 1˜Site 2>Site 4.

### Example 2

### Genetically Engineered T-CAR Cells

Gene cassettes were introduced at CRISPR break/insertion Sites 1, 2, 3 or 4 of the B2M gene described above. The DNA breaks inactivated the endogenous B2M gene, and allowed the introduction of the bicistronic gene cassettes of the present disclosure under the control of the native B2M promoter. The bicistronic constructs comprised a sequence encoding a non-functional portion of the B2M gene fused in frame with a polynucleotide encoding the human leukocyte antigen-E (HLA-E) molecule. The bicistronic constructs also comprised a polynucleotide sequence encoding a specific CAR molecule comprising an scFv derived from the anti-GD2 antibody dinutuximab (UNITUXIN®)

The absence of functional B2M expression in engineered T-cells may trigger an immune response. For that reason, the bicistronic constructs of the present disclose included a polynucleotide sequence encoding HLA-E. HLA-E is almost non-polymorphic, and is common to all humans. The expression of the construct encoding the partial but inactive B2M fused in frame with the HLA-E molecule allowed the immune system to sense that the genetically engineered cells, although not expressing B2M, were human and not a danger. As the CAR-T cell expressing the HLA-E molecule were not seen as foreign despite the lack of expression of functional B2M, they did not trigger an immune response against the therapeutic cells. Accordingly, such cells would be suitable for allogeneic therapy.

### Example 3

### Design of Chimeric Antigen Receptor (CAR) and B2M-HLA-E (ISMM) Elements

The bicistronic polynucleotide constructs of the present disclosure used in these experiments comprised a chimeric antigen receptor (CAR) and an Immune Surveillance Masking Molecule (ISMM). The CAR genetic element comprised a polynucleotide encoding, from N-terminus to C-terminus, the following operably linked elements
(1) an anti-GD2 scFv derived from dinutuximab;
(2) a CD8alpha hinge (here also referred as CD8a Hinge);
(3) a CD28 transmembrane domain (TM or CD28TM) and CD28 intracellular domain (CD28IC), which can be referred jointly as CD28TM/IC;
(4) a 4-1BB activation domain (4-1BBAD); and,
(5) a CD3zeta activation domain (CD3zetaAD).

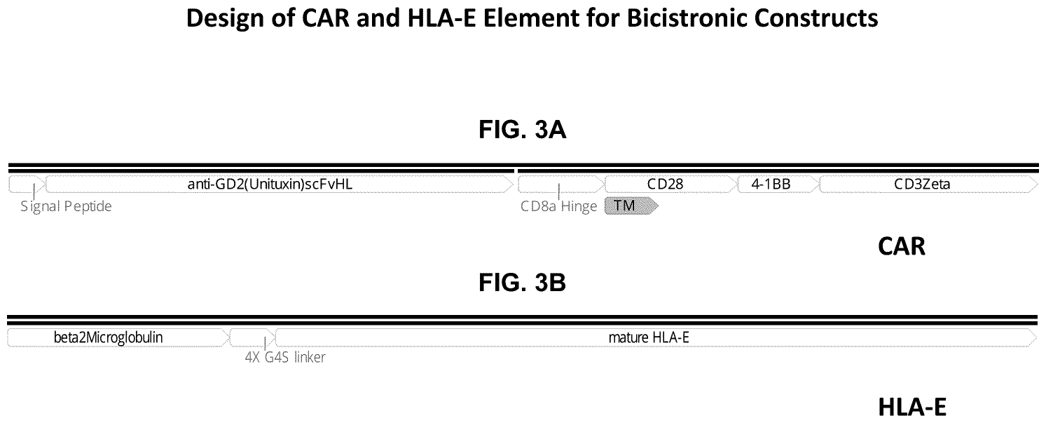

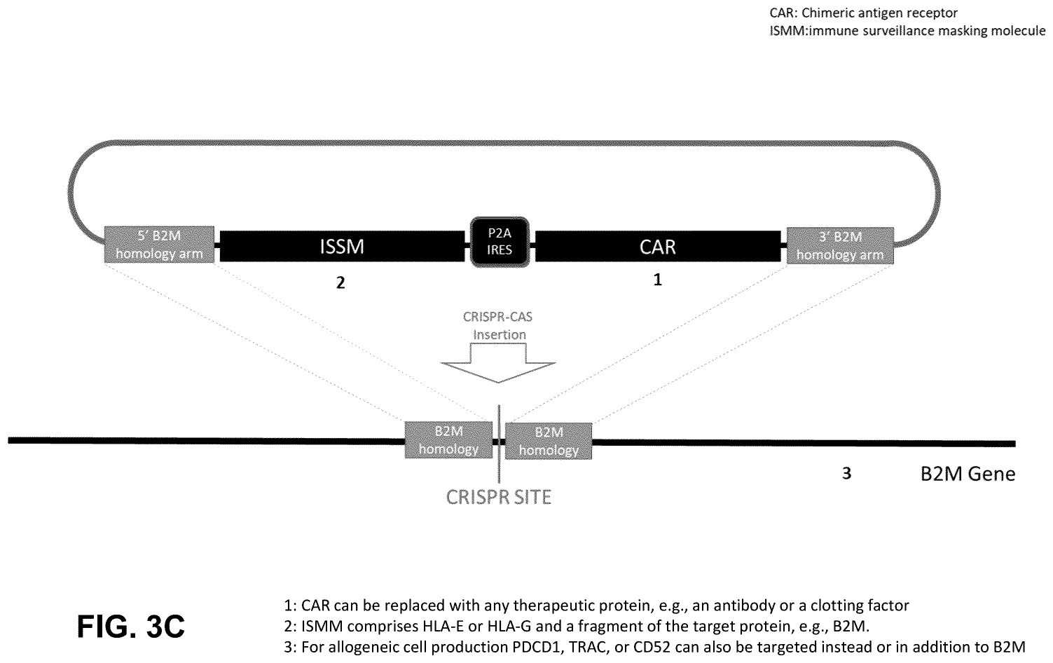

The structure of the CAR element of the bicistronic constructs, anti-GD2scFv+CD8aH+CD28TM/IC+4-1BBAD+CD3zetaAD (SEQ ID NO: 5), is shown in FIG. 3A.

>CAR
(SEQ ID NO: 5)
ATGGAACTGGGACTGTCATGGATCTTCTTGCTGGCTATCCTGAAGGGAGTGCAGTGTGAAGTTCAGCTGCTGCAGAGCGGACCCGA
ACTGGAAAAACCTGGCGCCTCCGTGATGATCAGCTGCAAGGCCTCTGGCAGCTCCTTCACCGGCTACAACATGAACTGGGTCCGAC
AGAACATCGGCAAGAGCCTGGAATGGATCGGCGCCATCGATCCTTACTACGGCGGCACCAGCTACAACCAGAAGTTCAAGGGCAGA
GCCACACTGACCGTGGACAAGAGCAGCAGCACAGCCTACATGCATCTGAAGTCCCTGACCAGCGAGGACAGCGCCGTGTACTACTG
TGTGTCCGGCATGGAATACTGGGGCCAGGGCACAAGCGTGACAGTCTCTTCTGGCGGCGGTGGATCTGGCGGAGGCGGAAGTGGTG
GCGGCGGATCTGATGTGGTCATGACACAGACCCCTCTGAGCCTGCCTGTGTCTCTGGGAGATCAGGCCAGCATCAGCTGTAGAAGC
AGCCAGAGCCTGGTGCACAGAAACGGCAACACCTACCTGCACTGGTATCTGCAGAAGCCCGGCCAGTCTCCTAAGCTGCTGATCCA
CAAGGTGTCCAACAGATTCAGCGGCGTGCCCGACAGATTCTCTGGCTCTGGAAGCGGCACCGACTTCACCCTGAAGATTAGCAGAG
TGGAAGCCGAGGACCTGGGCGTGTACTTCTGTAGCCAGAGCACACACGTGCCACCTCTGACATTTGGCGCTGGCACCAAGCTGGAA
CTGAAAAGGACCACGACGCCAGCGCCGCGACCACCAACACCGGCGCCCACCATCGCGTCGCAGCCCCTGTCCCTGCGCCCAGAGGC
GTGCCGGCCAGCGGCGGGGGGCGCAGTGCACACGAGGGGGCTGGACTTCGCCTGTGATTTTTGGGTGCTGGTGGTGGTTGGTGGAG
TCCTGGCTTGCTATAGCTTGCTAGTAACAGTGGCCTTTATTATTTTCTGGGTGAGGAGTAAGAGGAGCAGGCTCCTGCACAGTGAC
TACATGAACATGACTCCCCGCCGCCCCGGGCCCACCCGCAAGCATTACCAGCCCTATGCCCCACCACGCGACTTCGCAGCCTATCG
CTCCAAACGGGGCAGAAAGAAACTCCTGTATATATTCAAACAACCATTTATGAGACCAGTACAAACTACTCAAGAGGAAGATGGCT
GTAGCTGCCGATTTCCAGAAGAAGAAGAAGGAGGATGTGAACTGAGAGTGAAGTTCAGCAGGAGCGCAGACGCCCCCGCGTACCAG
CAGGGCCAGAACCAGCTCTATAACGAGCTCAATCTAGGACGAAGAGAGGAGTACGATGTTTTGGACAAGAGACGTGGCCGGGACCC
TGAGATGGGGGGAAAGCCGAGAAGGAAGAACCCTCAGGAAGGCCTGTACAATGAACTGCAGAAAGATAAGATGGCGGAGGCCTACA
GTGAGATTGGGATGAAAGGCGAGCGCCGGAGGGGCAAGGGGCACGATGGCCTTTACCAGGGTCTCAGTACAGCCACCAAGGACACC
TACGACGCCCTTCACATGCAGGCCCTGCCCCCTCGC

The ISMM genetic element of the bicistronic constructs of the present disclosure comprised a polynucleotide encoding a B2M inactive fragment fused in frame with a polypeptide encoding the mature human leukocyte antigen-E molecule (HLE-E), and a 4× glycine linker (Gly₄Ser)₄ interposed between the B2M and HLA-E portions of the molecule. The structure of the ISMM element of the bicistronic constructs, B2M+Gly-linker+HLA-E (SEQ ID NO: 6), is shown in FIG. 3B).

>ISMM B2M-HLA-E
(SEQ ID NO: 6)
ATCCAGCGTACTCCAAAGATTCAGGTTTACTCACGTCATCCAGCAGAGAATGGAAAGTCAAATTTCCTGAATTGCTATGTGTCTGG
GTTTCATCCATCCGACATTGAAGTTGACTTACTGAAGAATGGAGAGAGAATTGAAAAAGTGGAGCATTCAGACTTGTCTTTCAGCA
AGGACTGGTCTTTCTATCTCTTGTACTACACTGAATTCACCCCCACTGAAAAAGATGAGTATGCCTGCCGTGTGAACCATGTGACT
TTGTCACAGCCCAAGATAGTTAAGTGGGATCGAGACATGGGTGGAGGCGGTTCTGGAGGTGGCGGAAGTGGCGGCGGGGGAAGCGG
TGGGGGCGGTTCCGGCTCCCACTCCTTGAAGTATTTCCACACTTCCGTGTCCCGGCCCGGCCGCGGGGAGCCCCGCTTCATCTCTG
TGGGCTACGTGGACGACACCCAGTTCGTGCGCTTCGACAACGACGCCGCGAGTCCGAGGATGGTGCCGCGGGCGCCGTGGATGGAG
CAGGAGGGGTCAGAGTATTGGGACCGGGAGACACGGAGCGCCAGGGACACCGCACAGATTTTCCGAGTGAATCTGCGGACGCTGCG
CGGCTACTACAATCAGAGCGAGGCCGGGTCTCACACCCTGCAGTGGATGCATGGCTGCGAGCTGGGGCCCGACGGGCGCTTCCTCC
GCGGGTATGAACAGTTCGCCTACGACGGCAAGGATTATCTCACCCTGAATGAGGACCTGCGCTCCTGGACCGCGGTGGACACGGCG
GCTCAGATCTCCGAGCAAAAGTCAAATGATGCCTCTGAGGCGGAGCACCAGAGAGCCTACCTGGAAGACACATGCGTGGAGTGGCT
CCACAAATACCTGGAGAAGGGGAAGGAGACGCTGCTTCACCTGGAGCCCCCAAAGACACACGTGACTCACCACCCCATCTCTGACC
ATGAGGCCACCCTGAGGTGCTGGGCCCTGGGCTTCTACCCTGCGGAGATCACACTGACCTGGCAGCAGGATGGGGAGGGCCATACC
CAGGACACGGAGCTCGTGGAGACCAGGCCTGCAGGGGATGGAACCTTCCAGAAGTGGGCAGCTGTGGTGGTGCCTTCTGGAGAGGA
GCAGAGATACACGTGCCATGTGCAGCATGAGGGGCTACCCGAGCCCGTCACCCTGAGATGGAAGCCGGCTTCCCAGCCCACCATCC
CCATCGTGGGCATCATTGCTGGCCTGGTTCTCCTTGGATCTGTGGTCTCTGGAGCTGTGGTTGCTGCTGTGATATGGAGGAAGAAG
AGCTCAGGTGGAAAAGGAGGGAGCTACTCTAAGGCTGAGTGGAGCGACAGTGCCCAGGGGTCTGAGTCTCACAGCTTGTAA

### Example 4

### CAR-ISMM Bicistronic Construct and Full Donor Construct Design

Bicistronic constructs for insertion at the four B2M insertion sites described above were designed. Two different strategies were used to construct the bicistronic constructs. In a first strategy, a P2A cassette was introduced in frame between the CAR and ISMM components. The P2A element allowed the CAR and ISMM proteins to be separated during translation from a single mRNA. In a second strategy, an internal ribosome entry site (IRES) was introduced in frame between the CAR and ISMM components. The IRES element allowed the two fusion proteins to be independently translated from one single mRNA. Thus, in the first strategy, a single ribosome translated the entire bicistronic construct. In the second strategy, a first ribosome translated the CAR component and a second ribosome translated the ISMM element. In both cases a polyadenylation site (SV40 early polyadenylation signal) was introduced downstream from the 3′ end of the bicistronic construct to ensure that transcription stopped.

To avoid recognition and cleavage of the donor DNA sequence encoding the B2M gene by CRISPR reagents, silent mutations (dashed boxes in the schematic representations of the bicistronic constructs) were introduced in the donor B2M sequence. Silent mutations were introduced in the B2M encoding region of constructs inserted in Site 1, Site 2, and Site 3. No CRISPR-protecting silent mutations were introduced in the B2M encoding region of constructs inserted in Site 4.

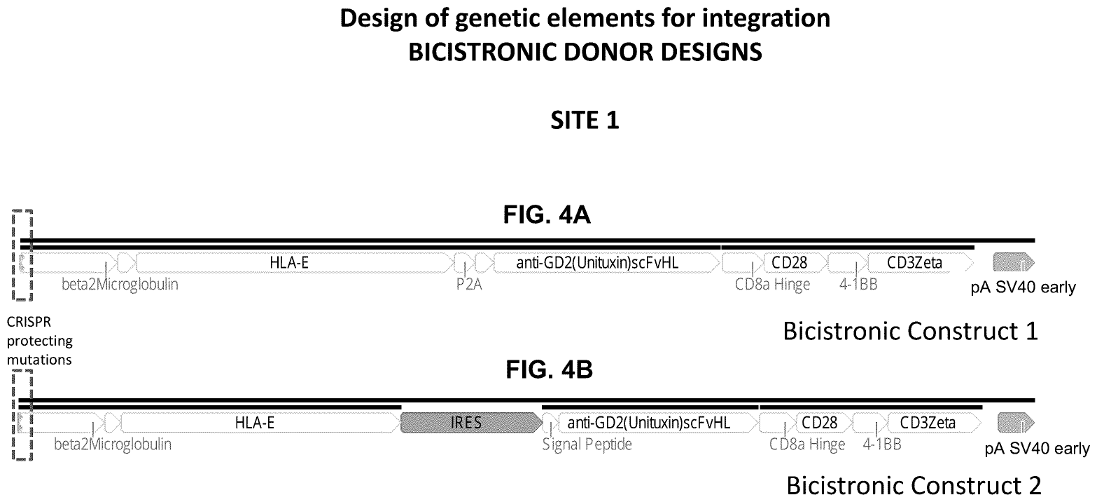

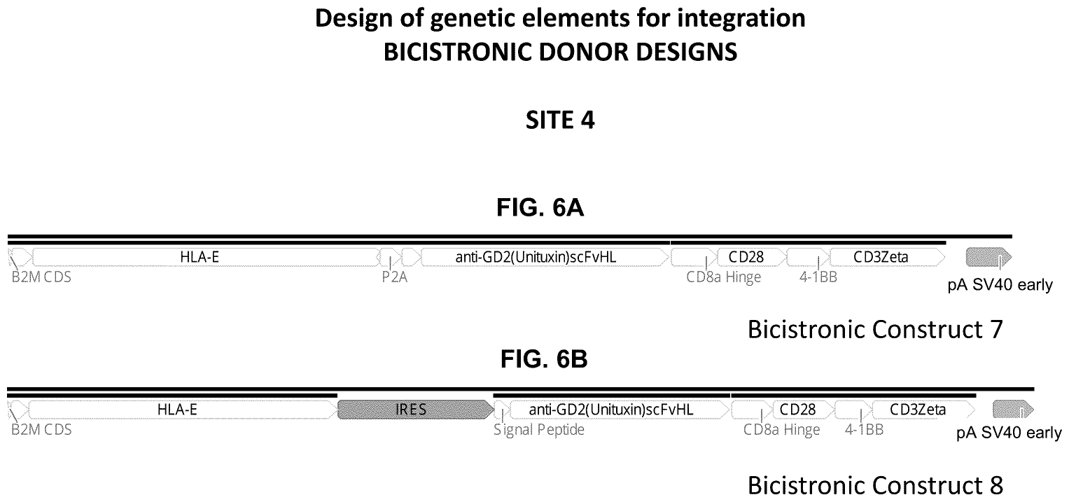

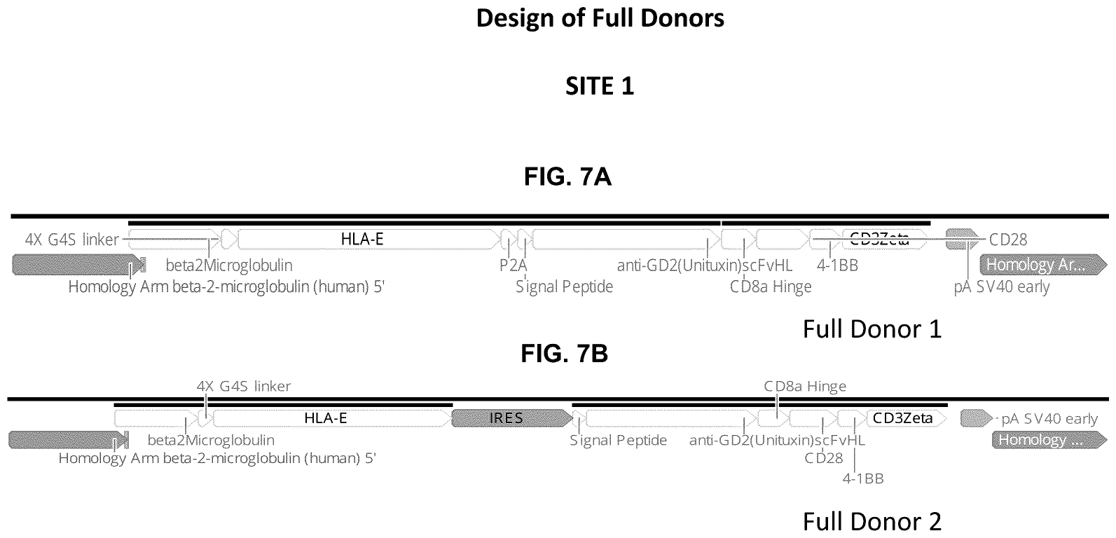

To achieve site-specific integration of the bicistronic donor sequence at the CRISPR reagents-induced DNA break, full donor constructs were generated in which 1000 bp of the B2M gene flanking the CRISPR sites (500 bp on each side of the CRISPR sites) were used to create homology arms to drive site specific recombination in frame (homology directed repair, or HDR). The full donor construct corresponding to the bicistronic constructs of FIG. 4A to FIG. 6B are depicted respectively in FIG. 7A to FIG. 9B, respectively.

The bicistronic constructs tested in the following examples as well as the full donor constructs are summarized in TABLE 1.

TABLE 1
Biscistronic constructs and donor contructs. “2A” indicates the presence of a P2A
between the CAR and ISMM components of the bicistronic construct. “IRES” indicates the
presence of an IRES between the CAR and ISMM components of the bicistronic construct. BC
Figure and BC Figure indicate the FIG. presenting the specific schematic presentation for
each one of the constructs.

| | | | | | Full | | |
|---|---|---|---|---|---|---|---|
| Bicistronic | B2M | | | | Donor | | |
| Construct | Insertion | Translation | BC | SEQ | Construct | FC | SEQ |
| (BC) | Site | Mechanism | Figure | ID NO | (FC) | Figure | ID NO |
| BC1 | 1 | 2A | 4A | 7 | FD1 | 7A | 13 |
| BC2 | 1 | IRES | 4B | 8 | FD2 | 7B | 14 |
| BC3 | 2 | 2A | 5A | 9 | FD3 | 8A | 15 |
| BC4 | 2 | IRES | 5B | 10 | FD4 | 8B | 16 |
| BC5 | 3 | 2A | 5A | 9 | FD5 | 8A | 15 |
| BC6 | 3 | IRES | 5B | 10 | FD6 | 8B | 16 |
| BC7 | 4 | 2A | 6A | 11 | FD7 | 9A | 17 |
| BC8 | 4 | IRES | 6B | 12 | FD8 | 9B | 18 |

>BC1 (SEQ ID NO: 7)
GCTAGAAGCTATCCAGCGTACTCCAAAGATTCAGGTTTACTCACGTCATCCAGCAGAGAATGGAAAGTCAAATTTCCTGAATTGCT
ATGTGTCTGGGTTTCATCCATCCGACATTGAAGTTGACTTACTGAAGAATGGAGAGAGAATTGAAAAAGTGGAGCATTCAGACTTG
TCTTTCAGCAAGGACTGGTCTTTCTATCTCTTGTACTACACTGAATTCACCCCCACTGAAAAAGATGAGTATGCCTGCCGTGTGAA
CCATGTGACTTTGTCACAGCCCAAGATAGTTAAGTGGGATCGAGACATGGGTGGAGGCGGTTCTGGAGGTGGCGGAAGTGGCGGCG
GGGGAAGCGGTGGGGGCGGTTCCGGCTCCCACTCCTTGAAGTATTTCCACACTTCCGTGTCCCGGCCCGGCCGCGGGGAGCCCCGC
TTCATCTCTGTGGGCTACGTGGACGACACCCAGTTCGTGCGCTTCGACAACGACGCCGCGAGTCCGAGGATGGTGCCGCGGGCGCC
GTGGATGGAGCAGGAGGGGTCAGAGTATTGGGACCGGGAGACACGGAGCGCCAGGGACACCGCACAGATTTTCCGAGTGAATCTGC
GGACGCTGCGCGGCTACTACAATCAGAGCGAGGCCGGGTCTCACACCCTGCAGTGGATGCATGGCTGCGAGCTGGGGCCCGACGGG
CGCTTCCTCCGCGGGTATGAACAGTTCGCCTACGACGGCAAGGATTATCTCACCCTGAATGAGGACCTGCGCTCCTGGACCGCGGT
GGACACGGCGGCTCAGATCTCCGAGCAAAAGTCAAATGATGCCTCTGAGGCGGAGCACCAGAGAGCCTACCTGGAAGACACATGCG
TGGAGTGGCTCCACAAATACCTGGAGAAGGGGAAGGAGACGCTGCTTCACCTGGAGCCCCCAAAGACACACGTGACTCACCACCCC
ATCTCTGACCATGAGGCCACCCTGAGGTGCTGGGCCCTGGGCTTCTACCCTGCGGAGATCACACTGACCTGGCAGCAGGATGGGGA
GGGCCATACCCAGGACACGGAGCTCGTGGAGACCAGGCCTGCAGGGGATGGAACCTTCCAGAAGTGGGCAGCTGTGGTGGTGCCTT
CTGGAGAGGAGCAGAGATACACGTGCCATGTGCAGCATGAGGGGCTACCCGAGCCCGTCACCCTGAGATGGAAGCCGGCTTCCCAG
CCCACCATCCCCATCGTGGGCATCATTGCTGGCCTGGTTCTCCTTGGATCTGTGGTCTCTGGAGCTGTGGTTGCTGCTGTGATATG
GAGGAAGAAGAGCTCAGGTGGAAAAGGAGGGAGCTACTCTAAGGCTGAGTGGAGCGACAGTGCCCAGGGGTCTGAGTCTCACAGCT
TGGGAAGCGGAGCTACTAACTTCAGCCTGCTGAAGCAGGCTGGAGACGTGGAGGAGAACCCTGGACCTATGGAACTGGGACTGTCA
TGGATCTTCTTGCTGGCTATCCTGAAGGGAGTGCAGTGTGAAGTTCAGCTGCTGCAGAGCGGACCCGAACTGGAAAAACCTGGCGC
CTCCGTGATGATCAGCTGCAAGGCCTCTGGCAGCTCCTTCACCGGCTACAACATGAACTGGGTCCGACAGAACATCGGCAAGAGCC
TGGAATGGATCGGCGCCATCGATCCTTACTACGGCGGCACCAGCTACAACCAGAAGTTCAAGGGCAGAGCCACACTGACCGTGGAC
AAGAGCAGCAGCACAGCCTACATGCATCTGAAGTCCCTGACCAGCGAGGACAGCGCCGTGTACTACTGTGTGTCCGGCATGGAATA
CTGGGGCCAGGGCACAAGCGTGACAGTCTCTTCTGGCGGCGGTGGATCTGGCGGAGGCGGAAGTGGTGGCGGCGGATCTGATGTGG
TCATGACACAGACCCCTCTGAGCCTGCCTGTGTCTCTGGGAGATCAGGCCAGCATCAGCTGTAGAAGCAGCCAGAGCCTGGTGCAC
AGAAACGGCAACACCTACCTGCACTGGTATCTGCAGAAGCCCGGCCAGTCTCCTAAGCTGCTGATCCACAAGGTGTCCAACAGATT
CAGCGGCGTGCCCGACAGATTCTCTGGCTCTGGAAGCGGCACCGACTTCACCCTGAAGATTAGCAGAGTGGAAGCCGAGGACCTGG
GCGTGTACTTCTGTAGCCAGAGCACACACGTGCCACCTCTGACATTTGGCGCTGGCACCAAGCTGGAACTGAAAAGGACCACGACG
CCAGCGCCGCGACCACCAACACCGGCGCCCACCATCGCGTCGCAGCCCCTGTCCCTGCGCCCAGAGGCGTGCCGGCCAGCGGCGGG
GGGCGCAGTGCACACGAGGGGGCTGGACTTCGCCTGTGATTTTTGGGTGCTGGTGGTGGTTGGTGGAGTCCTGGCTTGCTATAGCT
TGCTAGTAACAGTGGCCTTTATTATTTTCTGGGTGAGGAGTAAGAGGAGCAGGCTCCTGCACAGTGACTACATGAACATGACTCCC
CGCCGCCCCGGGCCCACCCGCAAGCATTACCAGCCCTATGCCCCACCACGCGACTTCGCAGCCTATCGCTCCAAACGGGGCAGAAA
GAAACTCCTGTATATATTCAAACAACCATTTATGAGACCAGTACAAACTACTCAAGAGGAAGATGGCTGTAGCTGCCGATTTCCAG
AAGAAGAAGAAGGAGGATGTGAACTGAGAGTGAAGTTCAGCAGGAGCGCAGACGCCCCCGCGTACCAGCAGGGCCAGAACCAGCTC
TATAACGAGCTCAATCTAGGACGAAGAGAGGAGTACGATGTTTTGGACAAGAGACGTGGCCGGGACCCTGAGATGGGGGGAAAGCC
GAGAAGGAAGAACCCTCAGGAAGGCCTGTACAATGAACTGCAGAAAGATAAGATGGCGGAGGCCTACAGTGAGATTGGGATGAAAG
GCGAGCGCCGGAGGGGCAAGGGGCACGATGGCCTTTACCAGGGTCTCAGTACAGCCACCAAGGACACCTACGACGCCCTTCACATG
CAGGCCCTGCCCCCTCGCTAGCTGCAGGGTCTGGCTGTTCTAGAGGCTGGGTTTGGGAGGTGAAACCTAGTGGCAGGAGAACTTGT
TTATTGCAGCTTATAATGGTTACAAATAAAGCAATAGCATCACAAATTTCACAAATAAAGCATTTTTTTCACTGCATTCTAGTTGT
GGTTTGTCCAAACTCATCAATGTATCTTATCATGTCTG
>B2 (SEQ ID NO: 8)
GCTAGAAGCTATCCAGCGTACTCCAAAGATTCAGGTTTACTCACGTCATCCAGCAGAGAATGGAAAGTCAAATTTCCTGAATTGCT
ATGTGTCTGGGTTTCATCCATCCGACATTGAAGTTGACTTACTGAAGAATGGAGAGAGAATTGAAAAAGTGGAGCATTCAGACTTG
TCTTTCAGCAAGGACTGGTCTTTCTATCTCTTGTACTACACTGAATTCACCCCCACTGAAAAAGATGAGTATGCCTGCCGTGTGAA
CCATGTGACTTTGTCACAGCCCAAGATAGTTAAGTGGGATCGAGACATGGGTGGAGGCGGTTCTGGAGGTGGCGGAAGTGGCGGCG
GGGGAAGCGGTGGGGGCGGTTCCGGCTCCCACTCCTTGAAGTATTTCCACACTTCCGTGTCCCGGCCCGGCCGCGGGGAGCCCCGC
TTCATCTCTGTGGGCTACGTGGACGACACCCAGTTCGTGCGCTTCGACAACGACGCCGCGAGTCCGAGGATGGTGCCGCGGGCGCC
GTGGATGGAGCAGGAGGGGTCAGAGTATTGGGACCGGGAGACACGGAGCGCCAGGGACACCGCACAGATTTTCCGAGTGAATCTGC
GGACGCTGCGCGGCTACTACAATCAGAGCGAGGCCGGGTCTCACACCCTGCAGTGGATGCATGGCTGCGAGCTGGGGCCCGACGGG
CGCTTCCTCCGCGGGTATGAACAGTTCGCCTACGACGGCAAGGATTATCTCACCCTGAATGAGGACCTGCGCTCCTGGACCGCGGT
GGACACGGCGGCTCAGATCTCCGAGCAAAAGTCAAATGATGCCTCTGAGGCGGAGCACCAGAGAGCCTACCTGGAAGACACATGCG
TGGAGTGGCTCCACAAATACCTGGAGAAGGGGAAGGAGACGCTGCTTCACCTGGAGCCCCCAAAGACACACGTGACTCACCACCCC
ATCTCTGACCATGAGGCCACCCTGAGGTGCTGGGCCCTGGGCTTCTACCCTGCGGAGATCACACTGACCTGGCAGCAGGATGGGGA
GGGCCATACCCAGGACACGGAGCTCGTGGAGACCAGGCCTGCAGGGGATGGAACCTTCCAGAAGTGGGCAGCTGTGGTGGTGCCTT
CTGGAGAGGAGCAGAGATACACGTGCCATGTGCAGCATGAGGGGCTACCCGAGCCCGTCACCCTGAGATGGAAGCCGGCTTCCCAG
CCCACCATCCCCATCGTGGGCATCATTGCTGGCCTGGTTCTCCTTGGATCTGTGGTCTCTGGAGCTGTGGTTGCTGCTGTGATATG
GAGGAAGAAGAGCTCAGGTGGAAAAGGAGGGAGCTACTCTAAGGCTGAGTGGAGCGACAGTGCCCAGGGGTCTGAGTCTCACAGCT
TGTAATCCGGTTATTTTCCACCATATTGCCGTCTTTTGGCAATGTGAGGGCCCGGAAACCTGGCCCTGTCTTCTTGACGAGCATTC
CTAGGGGTCTTTCCCCTCTCGCCAAAGGAATGCAAGGTCTGTTGAATGTCGTGAAGGAAGCAGTTCCTCTGGAAGCTTCTTGAAGA
CAAACAACGTCTGTAGCGACCCTTTGCAGGCAGCGGAACCCCCCACCTGGCGACAGGTGCCTCTGCGGCCAAAAGCCACGTGTATA
AGATACACCTGCAAAGGCGGCACAACCCCAGTGCCACGTTGTGAGTTGGATAGTTGTGGAAAGAGTCAAATGGCTCTCCTCAAGCG
TATTCAACAAGGGGCTGAAGGATGCCCAGAAGGTACCCCATTGTATGGGATCTGATCTGGGGCCTCGGTGCACATGCTTTACATGT
GTTTAGTCGAGGTTAAAAAACGTCTAGGCCCCCCGAACCACGGGGACGTGGTTTTCCTTTGAAAAACACGATGATAATAGGATCCA
TGATGGAACTGGGACTGTCATGGATCTTCTTGCTGGCTATCCTGAAGGGAGTGCAGTGTGAAGTTCAGCTGCTGCAGAGCGGACCC
GAACTGGAAAAACCTGGCGCCTCCGTGATGATCAGCTGCAAGGCCTCTGGCAGCTCCTTCACCGGCTACAACATGAACTGGGTCCG
ACAGAACATCGGCAAGAGCCTGGAATGGATCGGCGCCATCGATCCTTACTACGGCGGCACCAGCTACAACCAGAAGTTCAAGGGCA
GAGCCACACTGACCGTGGACAAGAGCAGCAGCACAGCCTACATGCATCTGAAGTCCCTGACCAGCGAGGACAGCGCCGTGTACTAC
TGTGTGTCCGGCATGGAATACTGGGGCCAGGGCACAAGCGTGACAGTCTCTTCTGGCGGCGGTGGATCTGGCGGAGGCGGAAGTGG
TGGCGGCGGATCTGATGTGGTCATGACACAGACCCCTCTGAGCCTGCCTGTGTCTCTGGGAGATCAGGCCAGCATCAGCTGTAGAA
GCAGCCAGAGCCTGGTGCACAGAAACGGCAACACCTACCTGCACTGGTATCTGCAGAAGCCCGGCCAGTCTCCTAAGCTGCTGATC
CACAAGGTGTCCAACAGATTCAGCGGCGTGCCCGACAGATTCTCTGGCTCTGGAAGCGGCACCGACTTCACCCTGAAGATTAGCAG
AGTGGAAGCCGAGGACCTGGGCGTGTACTTCTGTAGCCAGAGCACACACGTGCCACCTCTGACATTTGGCGCTGGCACCAAGCTGG
AACTGAAAAGGACCACGACGCCAGCGCCGCGACCACCAACACCGGCGCCCACCATCGCGTCGCAGCCCCTGTCCCTGCGCCCAGAG
GCGTGCCGGCCAGCGGCGGGGGGCGCAGTGCACACGAGGGGGCTGGACTTCGCCTGTGATTTTTGGGTGCTGGTGGTGGTTGGTGG
AGTCCTGGCTTGCTATAGCTTGCTAGTAACAGTGGCCTTTATTATTTTCTGGGTGAGGAGTAAGAGGAGCAGGCTCCTGCACAGTG
ACTACATGAACATGACTCCCCGCCGCCCCGGGCCCACCCGCAAGCATTACCAGCCCTATGCCCCACCACGCGACTTCGCAGCCTAT
CGCTCCAAACGGGGCAGAAAGAAACTCCTGTATATATTCAAACAACCATTTATGAGACCAGTACAAACTACTCAAGAGGAAGATGG
CTGTAGCTGCCGATTTCCAGAAGAAGAAGAAGGAGGATGTGAACTGAGAGTGAAGTTCAGCAGGAGCGCAGACGCCCCCGCGTACC
AGCAGGGCCAGAACCAGCTCTATAACGAGCTCAATCTAGGACGAAGAGAGGAGTACGATGTTTTGGACAAGAGACGTGGCCGGGAC
CCTGAGATGGGGGGAAAGCCGAGAAGGAAGAACCCTCAGGAAGGCCTGTACAATGAACTGCAGAAAGATAAGATGGCGGAGGCCTA
CAGTGAGATTGGGATGAAAGGCGAGCGCCGGAGGGGCAAGGGGCACGATGGCCTTTACCAGGGTCTCAGTACAGCCACCAAGGACA
CCTACGACGCCCTTCACATGCAGGCCCTGCCCCCTCGCTAGCTGCAGGGTCTGGCTGTTCTAGAGGCTGGGTTTGGGAGGTGAAAC
CTAGTGGCAGGAGAACTTGTTTATTGCAGCTTATAATGGTTACAAATAAAGCAATAGCATCACAAATTTCACAAATAAAGCATTTT
TTTCACTGCATTCTAGTTGTGGTTTGTCCAAACTCATCAATGTATCTTATCATGTCTG
>BC3/5 (SEQ ID NO: 9)
ATGCCGCGTGAACCATGTGACTTTGTCACAGCCCAAGATCGTGAAATGGGATCGAGACATGGGTGGAGGCGGTTCTGGAGGTGGCG
GAAGTGGCGGCGGGGGAAGCGGTGGGGGCGGTTCCGGCTCCCACTCCTTGAAGTATTTCCACACTTCCGTGTCCCGGCCCGGCCGC
GGGGAGCCCCGCTTCATCTCTGTGGGCTACGTGGACGACACCCAGTTCGTGCGCTTCGACAACGACGCCGCGAGTCCGAGGATGGT
GCCGCGGGCGCCGTGGATGGAGCAGGAGGGGTCAGAGTATTGGGACCGGGAGACACGGAGCGCCAGGGACACCGCACAGATTTTCC
GAGTGAATCTGCGGACGCTGCGCGGCTACTACAATCAGAGCGAGGCCGGGTCTCACACCCTGCAGTGGATGCATGGCTGCGAGCTG
GGGCCCGACGGGCGCTTCCTCCGCGGGTATGAACAGTTCGCCTACGACGGCAAGGATTATCTCACCCTGAATGAGGACCTGCGCTC
CTGGACCGCGGTGGACACGGCGGCTCAGATCTCCGAGCAAAAGTCAAATGATGCCTCTGAGGCGGAGCACCAGAGAGCCTACCTGG
AAGACACATGCGTGGAGTGGCTCCACAAATACCTGGAGAAGGGGAAGGAGACGCTGCTTCACCTGGAGCCCCCAAAGACACACGTG
ACTCACCACCCCATCTCTGACCATGAGGCCACCCTGAGGTGCTGGGCCCTGGGCTTCTACCCTGCGGAGATCACACTGACCTGGCA
GCAGGATGGGGAGGGCCATACCCAGGACACGGAGCTCGTGGAGACCAGGCCTGCAGGGGATGGAACCTTCCAGAAGTGGGCAGCTG
TGGTGGTGCCTTCTGGAGAGGAGCAGAGATACACGTGCCATGTGCAGCATGAGGGGCTACCCGAGCCCGTCACCCTGAGATGGAAG
CCGGCTTCCCAGCCCACCATCCCCATCGTGGGCATCATTGCTGGCCTGGTTCTCCTTGGATCTGTGGTCTCTGGAGCTGTGGTTGC
TGCTGTGATATGGAGGAAGAAGAGCTCAGGTGGAAAAGGAGGGAGCTACTCTAAGGCTGAGTGGAGCGACAGTGCCCAGGGGTCTG
AGTCTCACAGCTTGGGAAGCGGAGCTACTAACTTCAGCCTGCTGAAGCAGGCTGGAGACGTGGAGGAGAACCCTGGACCTATGGAA
CTGGGACTGTCATGGATCTTCTTGCTGGCTATCCTGAAGGGAGTGCAGTGTGAAGTTCAGCTGCTGCAGAGCGGACCCGAACTGGA
AAAACCTGGCGCCTCCGTGATGATCAGCTGCAAGGCCTCTGGCAGCTCCTTCACCGGCTACAACATGAACTGGGTCCGACAGAACA
TCGGCAAGAGCCTGGAATGGATCGGCGCCATCGATCCTTACTACGGCGGCACCAGCTACAACCAGAAGTTCAAGGGCAGAGCCACA
CTGACCGTGGACAAGAGCAGCAGCACAGCCTACATGCATCTGAAGTCCCTGACCAGCGAGGACAGCGCCGTGTACTACTGTGTGTC
CGGCATGGAATACTGGGGCCAGGGCACAAGCGTGACAGTCTCTTCTGGCGGCGGTGGATCTGGCGGAGGCGGAAGTGGTGGCGGCG
GATCTGATGTGGTCATGACACAGACCCCTCTGAGCCTGCCTGTGTCTCTGGGAGATCAGGCCAGCATCAGCTGTAGAAGCAGCCAG
AGCCTGGTGCACAGAAACGGCAACACCTACCTGCACTGGTATCTGCAGAAGCCCGGCCAGTCTCCTAAGCTGCTGATCCACAAGGT
GTCCAACAGATTCAGCGGCGTGCCCGACAGATTCTCTGGCTCTGGAAGCGGCACCGACTTCACCCTGAAGATTAGCAGAGTGGAAG
CCGAGGACCTGGGCGTGTACTTCTGTAGCCAGAGCACACACGTGCCACCTCTGACATTTGGCGCTGGCACCAAGCTGGAACTGAAA
AGGACCACGACGCCAGCGCCGCGACCACCAACACCGGCGCCCACCATCGCGTCGCAGCCCCTGTCCCTGCGCCCAGAGGCGTGCCG
GCCAGCGGCGGGGGGCGCAGTGCACACGAGGGGGCTGGACTTCGCCTGTGATTTTTGGGTGCTGGTGGTGGTTGGTGGAGTCCTGG
CTTGCTATAGCTTGCTAGTAACAGTGGCCTTTATTATTTTCTGGGTGAGGAGTAAGAGGAGCAGGCTCCTGCACAGTGACTACATG
AACATGACTCCCCGCCGCCCCGGGCCCACCCGCAAGCATTACCAGCCCTATGCCCCACCACGCGACTTCGCAGCCTATCGCTCCAA
ACGGGGCAGAAAGAAACTCCTGTATATATTCAAACAACCATTTATGAGACCAGTACAAACTACTCAAGAGGAAGATGGCTGTAGCT
GCCGATTTCCAGAAGAAGAAGAAGGAGGATGTGAACTGAGAGTGAAGTTCAGCAGGAGCGCAGACGCCCCCGCGTACCAGCAGGGC
CAGAACCAGCTCTATAACGAGCTCAATCTAGGACGAAGAGAGGAGTACGATGTTTTGGACAAGAGACGTGGCCGGGACCCTGAGAT
GGGGGGAAAGCCGAGAAGGAAGAACCCTCAGGAAGGCCTGTACAATGAACTGCAGAAAGATAAGATGGCGGAGGCCTACAGTGAGA
TTGGGATGAAAGGCGAGCGCCGGAGGGGCAAGGGGCACGATGGCCTTTACCAGGGTCTCAGTACAGCCACCAAGGACACCTACGAC
GCCCTTCACATGCAGGCCCTGCCCCCTCGCTAGCGGTCTGGCTGTTCTAGAGGCTGGGTTTGGGAGGTGAAACCTAGTGGCAGGAG
AACTTGTTTATTGCAGCTTATAATGGTTACAAATAAAGCAATAGCATCACAAATTTCACAAATAAAGCATTTTTTTCACTGCATTC
TAGTTGTGGTTTGTCCAAACTCATCAATGTATCTTATCATGTCTG
>BC4/6 (SEQ ID NO: 10)
ATGCCGCGTGAACCATGTGACTTTGTCACAGCCCAAGATCGTGAAATGGGATCGAGACATGGGTGGAGGCGGTTCTGGAGGTGGCG
GAAGTGGCGGCGGGGGAAGCGGTGGGGGCGGTTCCGGCTCCCACTCCTTGAAGTATTTCCACACTTCCGTGTCCCGGCCCGGCCGC
GGGGAGCCCCGCTTCATCTCTGTGGGCTACGTGGACGACACCCAGTTCGTGCGCTTCGACAACGACGCCGCGAGTCCGAGGATGGT
GCCGCGGGCGCCGTGGATGGAGCAGGAGGGGTCAGAGTATTGGGACCGGGAGACACGGAGCGCCAGGGACACCGCACAGATTTTCC
GAGTGAATCTGCGGACGCTGCGCGGCTACTACAATCAGAGCGAGGCCGGGTCTCACACCCTGCAGTGGATGCATGGCTGCGAGCTG
GGGCCCGACGGGCGCTTCCTCCGCGGGTATGAACAGTTCGCCTACGACGGCAAGGATTATCTCACCCTGAATGAGGACCTGCGCTC
CTGGACCGCGGTGGACACGGCGGCTCAGATCTCCGAGCAAAAGTCAAATGATGCCTCTGAGGCGGAGCACCAGAGAGCCTACCTGG
AAGACACATGCGTGGAGTGGCTCCACAAATACCTGGAGAAGGGGAAGGAGACGCTGCTTCACCTGGAGCCCCCAAAGACACACGTG
ACTCACCACCCCATCTCTGACCATGAGGCCACCCTGAGGTGCTGGGCCCTGGGCTTCTACCCTGCGGAGATCACACTGACCTGGCA
GCAGGATGGGGAGGGCCATACCCAGGACACGGAGCTCGTGGAGACCAGGCCTGCAGGGGATGGAACCTTCCAGAAGTGGGCAGCTG
TGGTGGTGCCTTCTGGAGAGGAGCAGAGATACACGTGCCATGTGCAGCATGAGGGGCTACCCGAGCCCGTCACCCTGAGATGGAAG
CCGGCTTCCCAGCCCACCATCCCCATCGTGGGCATCATTGCTGGCCTGGTTCTCCTTGGATCTGTGGTCTCTGGAGCTGTGGTTGC
TGCTGTGATATGGAGGAAGAAGAGCTCAGGTGGAAAAGGAGGGAGCTACTCTAAGGCTGAGTGGAGCGACAGTGCCCAGGGGTCTG
AGTCTCACAGCTTGTAATCCGGTTATTTTCCACCATATTGCCGTCTTTTGGCAATGTGAGGGCCCGGAAACCTGGCCCTGTCTTCT
TGACGAGCATTCCTAGGGGTCTTTCCCCTCTCGCCAAAGGAATGCAAGGTCTGTTGAATGTCGTGAAGGAAGCAGTTCCTCTGGAA
GCTTCTTGAAGACAAACAACGTCTGTAGCGACCCTTTGCAGGCAGCGGAACCCCCCACCTGGCGACAGGTGCCTCTGCGGCCAAAA
GCCACGTGTATAAGATACACCTGCAAAGGCGGCACAACCCCAGTGCCACGTTGTGAGTTGGATAGTTGTGGAAAGAGTCAAATGGC
TCTCCTCAAGCGTATTCAACAAGGGGCTGAAGGATGCCCAGAAGGTACCCCATTGTATGGGATCTGATCTGGGGCCTCGGTGCACA
TGCTTTACATGTGTTTAGTCGAGGTTAAAAAACGTCTAGGCCCCCCGAACCACGGGGACGTGGTTTTCCTTTGAAAAACACGATGA
TAATAGGATCCATGATGGAACTGGGACTGTCATGGATCTTCTTGCTGGCTATCCTGAAGGGAGTGCAGTGTGAAGTTCAGCTGCTG
CAGAGCGGACCCGAACTGGAAAAACCTGGCGCCTCCGTGATGATCAGCTGCAAGGCCTCTGGCAGCTCCTTCACCGGCTACAACAT
GAACTGGGTCCGACAGAACATCGGCAAGAGCCTGGAATGGATCGGCGCCATCGATCCTTACTACGGCGGCACCAGCTACAACCAGA
AGTTCAAGGGCAGAGCCACACTGACCGTGGACAAGAGCAGCAGCACAGCCTACATGCATCTGAAGTCCCTGACCAGCGAGGACAGC
GCCGTGTACTACTGTGTGTCCGGCATGGAATACTGGGGCCAGGGCACAAGCGTGACAGTCTCTTCTGGCGGCGGTGGATCTGGCGG
AGGCGGAAGTGGTGGCGGCGGATCTGATGTGGTCATGACACAGACCCCTCTGAGCCTGCCTGTGTCTCTGGGAGATCAGGCCAGCA
TCAGCTGTAGAAGCAGCCAGAGCCTGGTGCACAGAAACGGCAACACCTACCTGCACTGGTATCTGCAGAAGCCCGGCCAGTCTCCT
AAGCTGCTGATCCACAAGGTGTCCAACAGATTCAGCGGCGTGCCCGACAGATTCTCTGGCTCTGGAAGCGGCACCGACTTCACCCT
GAAGATTAGCAGAGTGGAAGCCGAGGACCTGGGCGTGTACTTCTGTAGCCAGAGCACACACGTGCCACCTCTGACATTTGGCGCTG
GCACCAAGCTGGAACTGAAAAGGACCACGACGCCAGCGCCGCGACCACCAACACCGGCGCCCACCATCGCGTCGCAGCCCCTGTCC
CTGCGCCCAGAGGCGTGCCGGCCAGCGGCGGGGGGCGCAGTGCACACGAGGGGGCTGGACTTCGCCTGTGATTTTTGGGTGCTGGT
GGTGGTTGGTGGAGTCCTGGCTTGCTATAGCTTGCTAGTAACAGTGGCCTTTATTATTTTCTGGGTGAGGAGTAAGAGGAGCAGGC
TCCTGCACAGTGACTACATGAACATGACTCCCCGCCGCCCCGGGCCCACCCGCAAGCATTACCAGCCCTATGCCCCACCACGCGAC
TTCGCAGCCTATCGCTCCAAACGGGGCAGAAAGAAACTCCTGTATATATTCAAACAACCATTTATGAGACCAGTACAAACTACTCA
AGAGGAAGATGGCTGTAGCTGCCGATTTCCAGAAGAAGAAGAAGGAGGATGTGAACTGAGAGTGAAGTTCAGCAGGAGCGCAGACG
CCCCCGCGTACCAGCAGGGCCAGAACCAGCTCTATAACGAGCTCAATCTAGGACGAAGAGAGGAGTACGATGTTTTGGACAAGAGA
CGTGGCCGGGACCCTGAGATGGGGGGAAAGCCGAGAAGGAAGAACCCTCAGGAAGGCCTGTACAATGAACTGCAGAAAGATAAGAT
GGCGGAGGCCTACAGTGAGATTGGGATGAAAGGCGAGCGCCGGAGGGGCAAGGGGCACGATGGCCTTTACCAGGGTCTCAGTACAG
CCACCAAGGACACCTACGACGCCCTTCACATGCAGGCCCTGCCCCCTCGCTAGCGGTCTGGCTGTTCTAGAGGCTGGGTTTGGGAG
GTGAAACCTAGTGGCAGGAGAACTTGTTTATTGCAGCTTATAATGGTTACAAATAAAGCAATAGCATCACAAATTTCACAAATAAA
GCATTTTTTTCACTGCATTCTAGTTGTGGTTTGTCCAAACTCATCAATGTATCTTATCATGTCTG
>BC7 (SEQ ID NO: 11)
ATCGAGACATGGGTGGAGGCGGTTCTGGAGGTGGCGGAAGTGGCGGCGGGGGAAGCGGTGGGGGCGGTTCCGGCTCCCACTCCTTG
AAGTATTTCCACACTTCCGTGTCCCGGCCCGGCCGCGGGGAGCCCCGCTTCATCTCTGTGGGCTACGTGGACGACACCCAGTTCGT
GCGCTTCGACAACGACGCCGCGAGTCCGAGGATGGTGCCGCGGGCGCCGTGGATGGAGCAGGAGGGGTCAGAGTATTGGGACCGGG
AGACACGGAGCGCCAGGGACACCGCACAGATTTTCCGAGTGAATCTGCGGACGCTGCGCGGCTACTACAATCAGAGCGAGGCCGGG
TCTCACACCCTGCAGTGGATGCATGGCTGCGAGCTGGGGCCCGACGGGCGCTTCCTCCGCGGGTATGAACAGTTCGCCTACGACGG
CAAGGATTATCTCACCCTGAATGAGGACCTGCGCTCCTGGACCGCGGTGGACACGGCGGCTCAGATCTCCGAGCAAAAGTCAAATG
ATGCCTCTGAGGCGGAGCACCAGAGAGCCTACCTGGAAGACACATGCGTGGAGTGGCTCCACAAATACCTGGAGAAGGGGAAGGAG
ACGCTGCTTCACCTGGAGCCCCCAAAGACACACGTGACTCACCACCCCATCTCTGACCATGAGGCCACCCTGAGGTGCTGGGCCCT
GGGCTTCTACCCTGCGGAGATCACACTGACCTGGCAGCAGGATGGGGAGGGCCATACCCAGGACACGGAGCTCGTGGAGACCAGGC
CTGCAGGGGATGGAACCTTCCAGAAGTGGGCAGCTGTGGTGGTGCCTTCTGGAGAGGAGCAGAGATACACGTGCCATGTGCAGCAT
GAGGGGCTACCCGAGCCCGTCACCCTGAGATGGAAGCCGGCTTCCCAGCCCACCATCCCCATCGTGGGCATCATTGCTGGCCTGGT
TCTCCTTGGATCTGTGGTCTCTGGAGCTGTGGTTGCTGCTGTGATATGGAGGAAGAAGAGCTCAGGTGGAAAAGGAGGGAGCTACT
CTAAGGCTGAGTGGAGCGACAGTGCCCAGGGGTCTGAGTCTCACAGCTTGGGAAGCGGAGCTACTAACTTCAGCCTGCTGAAGCAG
GCTGGAGACGTGGAGGAGAACCCTGGACCTATGGAACTGGGACTGTCATGGATCTTCTTGCTGGCTATCCTGAAGGGAGTGCAGTG
TGAAGTTCAGCTGCTGCAGAGCGGACCCGAACTGGAAAAACCTGGCGCCTCCGTGATGATCAGCTGCAAGGCCTCTGGCAGCTCCT
TCACCGGCTACAACATGAACTGGGTCCGACAGAACATCGGCAAGAGCCTGGAATGGATCGGCGCCATCGATCCTTACTACGGCGGC
ACCAGCTACAACCAGAAGTTCAAGGGCAGAGCCACACTGACCGTGGACAAGAGCAGCAGCACAGCCTACATGCATCTGAAGTCCCT
GACCAGCGAGGACAGCGCCGTGTACTACTGTGTGTCCGGCATGGAATACTGGGGCCAGGGCACAAGCGTGACAGTCTCTTCTGGCG
GCGGTGGATCTGGCGGAGGCGGAAGTGGTGGCGGCGGATCTGATGTGGTCATGACACAGACCCCTCTGAGCCTGCCTGTGTCTCTG
GGAGATCAGGCCAGCATCAGCTGTAGAAGCAGCCAGAGCCTGGTGCACAGAAACGGCAACACCTACCTGCACTGGTATCTGCAGAA
GCCCGGCCAGTCTCCTAAGCTGCTGATCCACAAGGTGTCCAACAGATTCAGCGGCGTGCCCGACAGATTCTCTGGCTCTGGAAGCG
GCACCGACTTCACCCTGAAGATTAGCAGAGTGGAAGCCGAGGACCTGGGCGTGTACTTCTGTAGCCAGAGCACACACGTGCCACCT
CTGACATTTGGCGCTGGCACCAAGCTGGAACTGAAAAGGACCACGACGCCAGCGCCGCGACCACCAACACCGGCGCCCACCATCGC
GTCGCAGCCCCTGTCCCTGCGCCCAGAGGCGTGCCGGCCAGCGGCGGGGGGCGCAGTGCACACGAGGGGGCTGGACTTCGCCTGTG
ATTTTTGGGTGCTGGTGGTGGTTGGTGGAGTCCTGGCTTGCTATAGCTTGCTAGTAACAGTGGCCTTTATTATTTTCTGGGTGAGG
AGTAAGAGGAGCAGGCTCCTGCACAGTGACTACATGAACATGACTCCCCGCCGCCCCGGGCCCACCCGCAAGCATTACCAGCCCTA
TGCCCCACCACGCGACTTCGCAGCCTATCGCTCCAAACGGGGCAGAAAGAAACTCCTGTATATATTCAAACAACCATTTATGAGAC
CAGTACAAACTACTCAAGAGGAAGATGGCTGTAGCTGCCGATTTCCAGAAGAAGAAGAAGGAGGATGTGAACTGAGAGTGAAGTTC
AGCAGGAGCGCAGACGCCCCCGCGTACCAGCAGGGCCAGAACCAGCTCTATAACGAGCTCAATCTAGGACGAAGAGAGGAGTACGA
TGTTTTGGACAAGAGACGTGGCCGGGACCCTGAGATGGGGGGAAAGCCGAGAAGGAAGAACCCTCAGGAAGGCCTGTACAATGAAC
TGCAGAAAGATAAGATGGCGGAGGCCTACAGTGAGATTGGGATGAAAGGCGAGCGCCGGAGGGGCAAGGGGCACGATGGCCTTTAC
CAGGGTCTCAGTACAGCCACCAAGGACACCTACGACGCCCTTCACATGCAGGCCCTGCCCCCTCGCTAGCTGCAGGGTCTGGCTGT
TCTAGAGGCTGGGTTTGGGAGGTGAAACCTAGTGGCAGGAGAACTTGTTTATTGCAGCTTATAATGGTTACAAATAAAGCAATAGC
ATCACAAATTTCACAAATAAAGCATTTTTTTCACTGCATTCTAGTTGTGGTTTGTCCAAACTCATCAATGTATCTTATCATGTCTG
>BC8 (SEQ ID NO: 12)
ATCGAGACATGGGTGGAGGCGGTTCTGGAGGTGGCGGAAGTGGCGGCGGGGGAAGCGGTGGGGGCGGTTCCGGCTCCCACTCCTTG
AAGTATTTCCACACTTCCGTGTCCCGGCCCGGCCGCGGGGAGCCCCGCTTCATCTCTGTGGGCTACGTGGACGACACCCAGTTCGT
GCGCTTCGACAACGACGCCGCGAGTCCGAGGATGGTGCCGCGGGCGCCGTGGATGGAGCAGGAGGGGTCAGAGTATTGGGACCGGG
AGACACGGAGCGCCAGGGACACCGCACAGATTTTCCGAGTGAATCTGCGGACGCTGCGCGGCTACTACAATCAGAGCGAGGCCGGG
TCTCACACCCTGCAGTGGATGCATGGCTGCGAGCTGGGGCCCGACGGGCGCTTCCTCCGCGGGTATGAACAGTTCGCCTACGACGG
CAAGGATTATCTCACCCTGAATGAGGACCTGCGCTCCTGGACCGCGGTGGACACGGCGGCTCAGATCTCCGAGCAAAAGTCAAATG
ATGCCTCTGAGGCGGAGCACCAGAGAGCCTACCTGGAAGACACATGCGTGGAGTGGCTCCACAAATACCTGGAGAAGGGGAAGGAG
ACGCTGCTTCACCTGGAGCCCCCAAAGACACACGTGACTCACCACCCCATCTCTGACCATGAGGCCACCCTGAGGTGCTGGGCCCT
GGGCTTCTACCCTGCGGAGATCACACTGACCTGGCAGCAGGATGGGGAGGGCCATACCCAGGACACGGAGCTCGTGGAGACCAGGC
CTGCAGGGGATGGAACCTTCCAGAAGTGGGCAGCTGTGGTGGTGCCTTCTGGAGAGGAGCAGAGATACACGTGCCATGTGCAGCAT
GAGGGGCTACCCGAGCCCGTCACCCTGAGATGGAAGCCGGCTTCCCAGCCCACCATCCCCATCGTGGGCATCATTGCTGGCCTGGT
TCTCCTTGGATCTGTGGTCTCTGGAGCTGTGGTTGCTGCTGTGATATGGAGGAAGAAGAGCTCAGGTGGAAAAGGAGGGAGCTACT
CTAAGGCTGAGTGGAGCGACAGTGCCCAGGGGTCTGAGTCTCACAGCTTGTAATCCGGTTATTTTCCACCATATTGCCGTCTTTTG
GCAATGTGAGGGCCCGGAAACCTGGCCCTGTCTTCTTGACGAGCATTCCTAGGGGTCTTTCCCCTCTCGCCAAAGGAATGCAAGGT
CTGTTGAATGTCGTGAAGGAAGCAGTTCCTCTGGAAGCTTCTTGAAGACAAACAACGTCTGTAGCGACCCTTTGCAGGCAGCGGAA
CCCCCCACCTGGCGACAGGTGCCTCTGCGGCCAAAAGCCACGTGTATAAGATACACCTGCAAAGGCGGCACAACCCCAGTGCCACG
TTGTGAGTTGGATAGTTGTGGAAAGAGTCAAATGGCTCTCCTCAAGCGTATTCAACAAGGGGCTGAAGGATGCCCAGAAGGTACCC
CATTGTATGGGATCTGATCTGGGGCCTCGGTGCACATGCTTTACATGTGTTTAGTCGAGGTTAAAAAACGTCTAGGCCCCCCGAAC
CACGGGGACGTGGTTTTCCTTTGAAAAACACGATGATAATAGGATCCATGATGGAACTGGGACTGTCATGGATCTTCTTGCTGGCT
ATCCTGAAGGGAGTGCAGTGTGAAGTTCAGCTGCTGCAGAGCGGACCCGAACTGGAAAAACCTGGCGCCTCCGTGATGATCAGCTG
CAAGGCCTCTGGCAGCTCCTTCACCGGCTACAACATGAACTGGGTCCGACAGAACATCGGCAAGAGCCTGGAATGGATCGGCGCCA
TCGATCCTTACTACGGCGGCACCAGCTACAACCAGAAGTTCAAGGGCAGAGCCACACTGACCGTGGACAAGAGCAGCAGCACAGCC
TACATGCATCTGAAGTCCCTGACCAGCGAGGACAGCGCCGTGTACTACTGTGTGTCCGGCATGGAATACTGGGGCCAGGGCACAAG
CGTGACAGTCTCTTCTGGCGGCGGTGGATCTGGCGGAGGCGGAAGTGGTGGCGGCGGATCTGATGTGGTCATGACACAGACCCCTC
TGAGCCTGCCTGTGTCTCTGGGAGATCAGGCCAGCATCAGCTGTAGAAGCAGCCAGAGCCTGGTGCACAGAAACGGCAACACCTAC
CTGCACTGGTATCTGCAGAAGCCCGGCCAGTCTCCTAAGCTGCTGATCCACAAGGTGTCCAACAGATTCAGCGGCGTGCCCGACAG
ATTCTCTGGCTCTGGAAGCGGCACCGACTTCACCCTGAAGATTAGCAGAGTGGAAGCCGAGGACCTGGGCGTGTACTTCTGTAGCC
AGAGCACACACGTGCCACCTCTGACATTTGGCGCTGGCACCAAGCTGGAACTGAAAAGGACCACGACGCCAGCGCCGCGACCACCA
ACACCGGCGCCCACCATCGCGTCGCAGCCCCTGTCCCTGCGCCCAGAGGCGTGCCGGCCAGCGGCGGGGGGCGCAGTGCACACGAG
GGGGCTGGACTTCGCCTGTGATTTTTGGGTGCTGGTGGTGGTTGGTGGAGTCCTGGCTTGCTATAGCTTGCTAGTAACAGTGGCCT
TTATTATTTTCTGGGTGAGGAGTAAGAGGAGCAGGCTCCTGCACAGTGACTACATGAACATGACTCCCCGCCGCCCCGGGCCCACC
CGCAAGCATTACCAGCCCTATGCCCCACCACGCGACTTCGCAGCCTATCGCTCCAAACGGGGCAGAAAGAAACTCCTGTATATATT
CAAACAACCATTTATGAGACCAGTACAAACTACTCAAGAGGAAGATGGCTGTAGCTGCCGATTTCCAGAAGAAGAAGAAGGAGGAT
GTGAACTGAGAGTGAAGTTCAGCAGGAGCGCAGACGCCCCCGCGTACCAGCAGGGCCAGAACCAGCTCTATAACGAGCTCAATCTA
GGACGAAGAGAGGAGTACGATGTTTTGGACAAGAGACGTGGCCGGGACCCTGAGATGGGGGGAAAGCCGAGAAGGAAGAACCCTCA
GGAAGGCCTGTACAATGAACTGCAGAAAGATAAGATGGCGGAGGCCTACAGTGAGATTGGGATGAAAGGCGAGCGCCGGAGGGGCA
AGGGGCACGATGGCCTTTACCAGGGTCTCAGTACAGCCACCAAGGACACCTACGACGCCCTTCACATGCAGGCCCTGCCCCCTCGC
TAGCTGCAGGGTCTGGCTGTTCTAGAGGCTGGGTTTGGGAGGTGAAACCTAGTGGCAGGAGAACTTGTTTATTGCAGCTTATAATG
GTTACAAATAAAGCAATAGCATCACAAATTTCACAAATAAAGCATTTTTTTCACTGCATTCTAGTTGTGGTTTGTCCAAACTCATC
AATGTATCTTATCATGTCTG
>FD1 (SEQ ID NO: 13)
AGATCTTAATCTTCTGGGTTTCCGTTTTCTCGAATGAAAAATGCAGGTCCGAGCAGTTAACTGGCTGGGGCACCATTAGCAAGTCA
CTTAGCATCTCTGGGGCCAGTCTGCAAAGCGAGGGGGCAGCCTTAATGTGCCTCCAGCCTGAAGTCCTAGAATGAGCGCCCGGTGT
CCCAAGCTGGGGCGCGCACCCCAGATCGGAGGGCGCCGATGTACAGACAGCAAACTCACCCAGTCTAGTGCATGCCTTCTTAAACA
TCACGAGACTCTAAGAAAAGGAAACTGAAAACGGGAAAGTCCCTCTCTCTAACCTGGCACTGCGTCGCTGGCTTGGAGACAGGTGA
CGGTCCCTGCGGGCCTTGTCCTGATTGGCTGGGCACGCGTTTAATATAAGTGGAGGCGTCGCGCTGGCGGGCATTCCTGAAGCTGA
CAGCATTCGGGCCGAGATGTCTCGCTCCGTGGCCTTAGCTGTGCTCGCGCTACTCTCTCTTTCTGGGCTAGAAGCTATCCAGCGTA
CTCCAAAGATTCAGGTTTACTCACGTCATCCAGCAGAGAATGGAAAGTCAAATTTCCTGAATTGCTATGTGTCTGGGTTTCATCCA
TCCGACATTGAAGTTGACTTACTGAAGAATGGAGAGAGAATTGAAAAAGTGGAGCATTCAGACTTGTCTTTCAGCAAGGACTGGTC
TTTCTATCTCTTGTACTACACTGAATTCACCCCCACTGAAAAAGATGAGTATGCCTGCCGTGTGAACCATGTGACTTTGTCACAGC
CCAAGATAGTTAAGTGGGATCGAGACATGGGTGGAGGCGGTTCTGGAGGTGGCGGAAGTGGCGGCGGGGGAAGCGGTGGGGGCGGT
TCCGGCTCCCACTCCTTGAAGTATTTCCACACTTCCGTGTCCCGGCCCGGCCGCGGGGAGCCCCGCTTCATCTCTGTGGGCTACGT
GGACGACACCCAGTTCGTGCGCTTCGACAACGACGCCGCGAGTCCGAGGATGGTGCCGCGGGCGCCGTGGATGGAGCAGGAGGGGT
CAGAGTATTGGGACCGGGAGACACGGAGCGCCAGGGACACCGCACAGATTTTCCGAGTGAATCTGCGGACGCTGCGCGGCTACTAC
AATCAGAGCGAGGCCGGGTCTCACACCCTGCAGTGGATGCATGGCTGCGAGCTGGGGCCCGACGGGCGCTTCCTCCGCGGGTATGA
ACAGTTCGCCTACGACGGCAAGGATTATCTCACCCTGAATGAGGACCTGCGCTCCTGGACCGCGGTGGACACGGCGGCTCAGATCT
CCGAGCAAAAGTCAAATGATGCCTCTGAGGCGGAGCACCAGAGAGCCTACCTGGAAGACACATGCGTGGAGTGGCTCCACAAATAC
CTGGAGAAGGGGAAGGAGACGCTGCTTCACCTGGAGCCCCCAAAGACACACGTGACTCACCACCCCATCTCTGACCATGAGGCCAC
CCTGAGGTGCTGGGCCCTGGGCTTCTACCCTGCGGAGATCACACTGACCTGGCAGCAGGATGGGGAGGGCCATACCCAGGACACGG
AGCTCGTGGAGACCAGGCCTGCAGGGGATGGAACCTTCCAGAAGTGGGCAGCTGTGGTGGTGCCTTCTGGAGAGGAGCAGAGATAC
ACGTGCCATGTGCAGCATGAGGGGCTACCCGAGCCCGTCACCCTGAGATGGAAGCCGGCTTCCCAGCCCACCATCCCCATCGTGGG
CATCATTGCTGGCCTGGTTCTCCTTGGATCTGTGGTCTCTGGAGCTGTGGTTGCTGCTGTGATATGGAGGAAGAAGAGCTCAGGTG
GAAAAGGAGGGAGCTACTCTAAGGCTGAGTGGAGCGACAGTGCCCAGGGGTCTGAGTCTCACAGCTTGGGAAGCGGAGCTACTAAC
TTCAGCCTGCTGAAGCAGGCTGGAGACGTGGAGGAGAACCCTGGACCTATGGAACTGGGACTGTCATGGATCTTCTTGCTGGCTAT
CCTGAAGGGAGTGCAGTGTGAAGTTCAGCTGCTGCAGAGCGGACCCGAACTGGAAAAACCTGGCGCCTCCGTGATGATCAGCTGCA
AGGCCTCTGGCAGCTCCTTCACCGGCTACAACATGAACTGGGTCCGACAGAACATCGGCAAGAGCCTGGAATGGATCGGCGCCATC
GATCCTTACTACGGCGGCACCAGCTACAACCAGAAGTTCAAGGGCAGAGCCACACTGACCGTGGACAAGAGCAGCAGCACAGCCTA
CATGCATCTGAAGTCCCTGACCAGCGAGGACAGCGCCGTGTACTACTGTGTGTCCGGCATGGAATACTGGGGCCAGGGCACAAGCG
TGACAGTCTCTTCTGGCGGCGGTGGATCTGGCGGAGGCGGAAGTGGTGGCGGCGGATCTGATGTGGTCATGACACAGACCCCTCTG
AGCCTGCCTGTGTCTCTGGGAGATCAGGCCAGCATCAGCTGTAGAAGCAGCCAGAGCCTGGTGCACAGAAACGGCAACACCTACCT
GCACTGGTATCTGCAGAAGCCCGGCCAGTCTCCTAAGCTGCTGATCCACAAGGTGTCCAACAGATTCAGCGGCGTGCCCGACAGAT
TCTCTGGCTCTGGAAGCGGCACCGACTTCACCCTGAAGATTAGCAGAGTGGAAGCCGAGGACCTGGGCGTGTACTTCTGTAGCCAG
AGCACACACGTGCCACCTCTGACATTTGGCGCTGGCACCAAGCTGGAACTGAAAAGGACCACGACGCCAGCGCCGCGACCACCAAC
ACCGGCGCCCACCATCGCGTCGCAGCCCCTGTCCCTGCGCCCAGAGGCGTGCCGGCCAGCGGCGGGGGGCGCAGTGCACACGAGGG
GGCTGGACTTCGCCTGTGATTTTTGGGTGCTGGTGGTGGTTGGTGGAGTCCTGGCTTGCTATAGCTTGCTAGTAACAGTGGCCTTT
ATTATTTTCTGGGTGAGGAGTAAGAGGAGCAGGCTCCTGCACAGTGACTACATGAACATGACTCCCCGCCGCCCCGGGCCCACCCG
CAAGCATTACCAGCCCTATGCCCCACCACGCGACTTCGCAGCCTATCGCTCCAAACGGGGCAGAAAGAAACTCCTGTATATATTCA
AACAACCATTTATGAGACCAGTACAAACTACTCAAGAGGAAGATGGCTGTAGCTGCCGATTTCCAGAAGAAGAAGAAGGAGGATGT
GAACTGAGAGTGAAGTTCAGCAGGAGCGCAGACGCCCCCGCGTACCAGCAGGGCCAGAACCAGCTCTATAACGAGCTCAATCTAGG
ACGAAGAGAGGAGTACGATGTTTTGGACAAGAGACGTGGCCGGGACCCTGAGATGGGGGGAAAGCCGAGAAGGAAGAACCCTCAGG
AAGGCCTGTACAATGAACTGCAGAAAGATAAGATGGCGGAGGCCTACAGTGAGATTGGGATGAAAGGCGAGCGCCGGAGGGGCAAG
GGGCACGATGGCCTTTACCAGGGTCTCAGTACAGCCACCAAGGACACCTACGACGCCCTTCACATGCAGGCCCTGCCCCCTCGCTA
GCTGCAGGGTCTGGCTGTTCTAGAGGCTGGGTTTGGGAGGTGAAACCTAGTGGCAGGAGAACTTGTTTATTGCAGCTTATAATGGT
TACAAATAAAGCAATAGCATCACAAATTTCACAAATAAAGCATTTTTTTCACTGCATTCTAGTTGTGGTTTGTCCAAACTCATCAA
TGTATCTTATCATGTCTGGAGGCTATCCAGCGTGAGTCTCTCCTACCCTCCCGCTCTGGTCCTTCCTCTCCCGCTCTGCACCCTCT
GTGGCCCTCGCTGTGCTCTCTCGCTCCGTGACTTCCCTTCTCCAAGTTCTCCTTGGTGGCCCGCCGTGGGGCTAGTCCAGGGCTGG
ATCTCGGGGAAGCGGCGGGGTGGCCTGGGAGTGGGGAAGGGGGTGCGCACCCGGGACGCGCGCTACTTGCCCCTTTCGGCGGGGAG
CAGGGGAGACCTTTGGCCTACGGCGACGGGAGGGTCGGGACAAAGTTTAGGGCGTCGATAAGCGTCAGAGCGCCGAGGTTGGGGGA
GGGTTTCTCTTCCGCTCTTTCGCGGGGCCTCTGGCTCCCCCAGCGCAGCTGGAGTGGGGGACGGGTAGGCTCGTCCCAAAGGCGCG
GCGCTGAGGTTTGTGAACGCGTGGAGGGGCGCTTGGGGTCTGGGGGAGGCGTCGCCCGGGTAAGCCTGTCTGCT
>FD2 (SEQ ID NO: 14)
AGATCTTAATCTTCTGGGTTTCCGTTTTCTCGAATGAAAAATGCAGGTCCGAGCAGTTAACTGGCTGGGGCACCATTAGCAAGTCA
CTTAGCATCTCTGGGGCCAGTCTGCAAAGCGAGGGGGCAGCCTTAATGTGCCTCCAGCCTGAAGTCCTAGAATGAGCGCCCGGTGT
CCCAAGCTGGGGCGCGCACCCCAGATCGGAGGGCGCCGATGTACAGACAGCAAACTCACCCAGTCTAGTGCATGCCTTCTTAAACA
TCACGAGACTCTAAGAAAAGGAAACTGAAAACGGGAAAGTCCCTCTCTCTAACCTGGCACTGCGTCGCTGGCTTGGAGACAGGTGA
CGGTCCCTGCGGGCCTTGTCCTGATTGGCTGGGCACGCGTTTAATATAAGTGGAGGCGTCGCGCTGGCGGGCATTCCTGAAGCTGA
CAGCATTCGGGCCGAGATGTCTCGCTCCGTGGCCTTAGCTGTGCTCGCGCTACTCTCTCTTTCTGGGCTAGAAGCTATCCAGCGTA
CTCCAAAGATTCAGGTTTACTCACGTCATCCAGCAGAGAATGGAAAGTCAAATTTCCTGAATTGCTATGTGTCTGGGTTTCATCCA
TCCGACATTGAAGTTGACTTACTGAAGAATGGAGAGAGAATTGAAAAAGTGGAGCATTCAGACTTGTCTTTCAGCAAGGACTGGTC
TTTCTATCTCTTGTACTACACTGAATTCACCCCCACTGAAAAAGATGAGTATGCCTGCCGTGTGAACCATGTGACTTTGTCACAGC
CCAAGATAGTTAAGTGGGATCGAGACATGGGTGGAGGCGGTTCTGGAGGTGGCGGAAGTGGCGGCGGGGGAAGCGGTGGGGGCGGT
TCCGGCTCCCACTCCTTGAAGTATTTCCACACTTCCGTGTCCCGGCCCGGCCGCGGGGAGCCCCGCTTCATCTCTGTGGGCTACGT
GGACGACACCCAGTTCGTGCGCTTCGACAACGACGCCGCGAGTCCGAGGATGGTGCCGCGGGCGCCGTGGATGGAGCAGGAGGGGT
CAGAGTATTGGGACCGGGAGACACGGAGCGCCAGGGACACCGCACAGATTTTCCGAGTGAATCTGCGGACGCTGCGCGGCTACTAC
AATCAGAGCGAGGCCGGGTCTCACACCCTGCAGTGGATGCATGGCTGCGAGCTGGGGCCCGACGGGCGCTTCCTCCGCGGGTATGA
ACAGTTCGCCTACGACGGCAAGGATTATCTCACCCTGAATGAGGACCTGCGCTCCTGGACCGCGGTGGACACGGCGGCTCAGATCT
CCGAGCAAAAGTCAAATGATGCCTCTGAGGCGGAGCACCAGAGAGCCTACCTGGAAGACACATGCGTGGAGTGGCTCCACAAATAC
CTGGAGAAGGGGAAGGAGACGCTGCTTCACCTGGAGCCCCCAAAGACACACGTGACTCACCACCCCATCTCTGACCATGAGGCCAC
CCTGAGGTGCTGGGCCCTGGGCTTCTACCCTGCGGAGATCACACTGACCTGGCAGCAGGATGGGGAGGGCCATACCCAGGACACGG
AGCTCGTGGAGACCAGGCCTGCAGGGGATGGAACCTTCCAGAAGTGGGCAGCTGTGGTGGTGCCTTCTGGAGAGGAGCAGAGATAC
ACGTGCCATGTGCAGCATGAGGGGCTACCCGAGCCCGTCACCCTGAGATGGAAGCCGGCTTCCCAGCCCACCATCCCCATCGTGGG
CATCATTGCTGGCCTGGTTCTCCTTGGATCTGTGGTCTCTGGAGCTGTGGTTGCTGCTGTGATATGGAGGAAGAAGAGCTCAGGTG
GAAAAGGAGGGAGCTACTCTAAGGCTGAGTGGAGCGACAGTGCCCAGGGGTCTGAGTCTCACAGCTTGTAATCCGGTTATTTTCCA
CCATATTGCCGTCTTTTGGCAATGTGAGGGCCCGGAAACCTGGCCCTGTCTTCTTGACGAGCATTCCTAGGGGTCTTTCCCCTCTC
GCCAAAGGAATGCAAGGTCTGTTGAATGTCGTGAAGGAAGCAGTTCCTCTGGAAGCTTCTTGAAGACAAACAACGTCTGTAGCGAC
CCTTTGCAGGCAGCGGAACCCCCCACCTGGCGACAGGTGCCTCTGCGGCCAAAAGCCACGTGTATAAGATACACCTGCAAAGGCGG
CACAACCCCAGTGCCACGTTGTGAGTTGGATAGTTGTGGAAAGAGTCAAATGGCTCTCCTCAAGCGTATTCAACAAGGGGCTGAAG
GATGCCCAGAAGGTACCCCATTGTATGGGATCTGATCTGGGGCCTCGGTGCACATGCTTTACATGTGTTTAGTCGAGGTTAAAAAA
CGTCTAGGCCCCCCGAACCACGGGGACGTGGTTTTCCTTTGAAAAACACGATGATAATAGGATCCATGATGGAACTGGGACTGTCA
TGGATCTTCTTGCTGGCTATCCTGAAGGGAGTGCAGTGTGAAGTTCAGCTGCTGCAGAGCGGACCCGAACTGGAAAAACCTGGCGC
CTCCGTGATGATCAGCTGCAAGGCCTCTGGCAGCTCCTTCACCGGCTACAACATGAACTGGGTCCGACAGAACATCGGCAAGAGCC
TGGAATGGATCGGCGCCATCGATCCTTACTACGGCGGCACCAGCTACAACCAGAAGTTCAAGGGCAGAGCCACACTGACCGTGGAC
AAGAGCAGCAGCACAGCCTACATGCATCTGAAGTCCCTGACCAGCGAGGACAGCGCCGTGTACTACTGTGTGTCCGGCATGGAATA
CTGGGGCCAGGGCACAAGCGTGACAGTCTCTTCTGGCGGCGGTGGATCTGGCGGAGGCGGAAGTGGTGGCGGCGGATCTGATGTGG
TCATGACACAGACCCCTCTGAGCCTGCCTGTGTCTCTGGGAGATCAGGCCAGCATCAGCTGTAGAAGCAGCCAGAGCCTGGTGCAC
AGAAACGGCAACACCTACCTGCACTGGTATCTGCAGAAGCCCGGCCAGTCTCCTAAGCTGCTGATCCACAAGGTGTCCAACAGATT
CAGCGGCGTGCCCGACAGATTCTCTGGCTCTGGAAGCGGCACCGACTTCACCCTGAAGATTAGCAGAGTGGAAGCCGAGGACCTGG
GCGTGTACTTCTGTAGCCAGAGCACACACGTGCCACCTCTGACATTTGGCGCTGGCACCAAGCTGGAACTGAAAAGGACCACGACG
CCAGCGCCGCGACCACCAACACCGGCGCCCACCATCGCGTCGCAGCCCCTGTCCCTGCGCCCAGAGGCGTGCCGGCCAGCGGCGGG
GGGCGCAGTGCACACGAGGGGGCTGGACTTCGCCTGTGATTTTTGGGTGCTGGTGGTGGTTGGTGGAGTCCTGGCTTGCTATAGCT
TGCTAGTAACAGTGGCCTTTATTATTTTCTGGGTGAGGAGTAAGAGGAGCAGGCTCCTGCACAGTGACTACATGAACATGACTCCC
CGCCGCCCCGGGCCCACCCGCAAGCATTACCAGCCCTATGCCCCACCACGCGACTTCGCAGCCTATCGCTCCAAACGGGGCAGAAA
GAAACTCCTGTATATATTCAAACAACCATTTATGAGACCAGTACAAACTACTCAAGAGGAAGATGGCTGTAGCTGCCGATTTCCAG
AAGAAGAAGAAGGAGGATGTGAACTGAGAGTGAAGTTCAGCAGGAGCGCAGACGCCCCCGCGTACCAGCAGGGCCAGAACCAGCTC
TATAACGAGCTCAATCTAGGACGAAGAGAGGAGTACGATGTTTTGGACAAGAGACGTGGCCGGGACCCTGAGATGGGGGGAAAGCC
GAGAAGGAAGAACCCTCAGGAAGGCCTGTACAATGAACTGCAGAAAGATAAGATGGCGGAGGCCTACAGTGAGATTGGGATGAAAG
GCGAGCGCCGGAGGGGCAAGGGGCACGATGGCCTTTACCAGGGTCTCAGTACAGCCACCAAGGACACCTACGACGCCCTTCACATG
CAGGCCCTGCCCCCTCGCTAGCTGCAGGGTCTGGCTGTTCTAGAGGCTGGGTTTGGGAGGTGAAACCTAGTGGCAGGAGAACTTGT
TTATTGCAGCTTATAATGGTTACAAATAAAGCAATAGCATCACAAATTTCACAAATAAAGCATTTTTTTCACTGCATTCTAGTTGT
GGTTTGTCCAAACTCATCAATGTATCTTATCATGTCTGGAGGCTATCCAGCGTGAGTCTCTCCTACCCTCCCGCTCTGGTCCTTCC
TCTCCCGCTCTGCACCCTCTGTGGCCCTCGCTGTGCTCTCTCGCTCCGTGACTTCCCTTCTCCAAGTTCTCCTTGGTGGCCCGCCG
TGGGGCTAGTCCAGGGCTGGATCTCGGGGAAGCGGCGGGGTGGCCTGGGAGTGGGGAAGGGGGTGCGCACCCGGGACGCGCGCTAC
TTGCCCCTTTCGGCGGGGAGCAGGGGAGACCTTTGGCCTACGGCGACGGGAGGGTCGGGACAAAGTTTAGGGCGTCGATAAGCGTC
AGAGCGCCGAGGTTGGGGGAGGGTTTCTCTTCCGCTCTTTCGCGGGGCCTCTGGCTCCCCCAGCGCAGCTGGAGTGGGGGACGGGT
AGGCTCGTCCCAAAGGCGCGGCGCTGAGGTTTGTGAACGCGTGGAGGGGCGCTTGGGGTCTGGGGGAGGCGTCGCCCGGGTAAGCC
TGTCTGCT
>FD3/5 (SEQ ID NO: 15)
TCTGCTAGAAAAAAAACAAAAAAGGCATGTATAGAGGAATTATGAGGGAAAGATACCAAGTCACGGTTTATTCTTCAAAATGGAGG
TGGCTTGTTGGGAAGGTGGAAGCTCATTTGGCCAGAGTGGAAATGGAATTGGGAGAAATCGATGACCAAATGTAAACACTTGGTGC
CTGATATAGCTTGACACCAAGTTAGCCCCAAGTGAAATACCCTGGCAATATTAATGTGTCTTTTCCCGATATTCCTCAGGTACTCC
AAAGATTCAGGTTTACTCACGTCATCCAGCAGAGAATGGAAAGTCAAATTTCCTGAATTGCTATGTGTCTGGGTTTCATCCATCCG
ACATTGAAGTTGACTTACTGAAGAATGGAGAGAGAATTGAAAAAGTGGAGCATTCAGACTTGTCTTTCAGCAAGGACTGGTCTTTC
TATCTCTTGTACTACACTGAATTCACCCCCACTGAAAAAGATGAGTATGCATGCCGCGTGAACCATGTGACTTTGTCACAGCCCAA
GATCGTGAAATGGGATCGAGACATGGGTGGAGGCGGTTCTGGAGGTGGCGGAAGTGGCGGCGGGGGAAGCGGTGGGGGCGGTTCCG
GCTCCCACTCCTTGAAGTATTTCCACACTTCCGTGTCCCGGCCCGGCCGCGGGGAGCCCCGCTTCATCTCTGTGGGCTACGTGGAC
GACACCCAGTTCGTGCGCTTCGACAACGACGCCGCGAGTCCGAGGATGGTGCCGCGGGCGCCGTGGATGGAGCAGGAGGGGTCAGA
GTATTGGGACCGGGAGACACGGAGCGCCAGGGACACCGCACAGATTTTCCGAGTGAATCTGCGGACGCTGCGCGGCTACTACAATC
AGAGCGAGGCCGGGTCTCACACCCTGCAGTGGATGCATGGCTGCGAGCTGGGGCCCGACGGGCGCTTCCTCCGCGGGTATGAACAG
TTCGCCTACGACGGCAAGGATTATCTCACCCTGAATGAGGACCTGCGCTCCTGGACCGCGGTGGACACGGCGGCTCAGATCTCCGA
GCAAAAGTCAAATGATGCCTCTGAGGCGGAGCACCAGAGAGCCTACCTGGAAGACACATGCGTGGAGTGGCTCCACAAATACCTGG
AGAAGGGGAAGGAGACGCTGCTTCACCTGGAGCCCCCAAAGACACACGTGACTCACCACCCCATCTCTGACCATGAGGCCACCCTG
AGGTGCTGGGCCCTGGGCTTCTACCCTGCGGAGATCACACTGACCTGGCAGCAGGATGGGGAGGGCCATACCCAGGACACGGAGCT
CGTGGAGACCAGGCCTGCAGGGGATGGAACCTTCCAGAAGTGGGCAGCTGTGGTGGTGCCTTCTGGAGAGGAGCAGAGATACACGT
GCCATGTGCAGCATGAGGGGCTACCCGAGCCCGTCACCCTGAGATGGAAGCCGGCTTCCCAGCCCACCATCCCCATCGTGGGCATC
ATTGCTGGCCTGGTTCTCCTTGGATCTGTGGTCTCTGGAGCTGTGGTTGCTGCTGTGATATGGAGGAAGAAGAGCTCAGGTGGAAA
AGGAGGGAGCTACTCTAAGGCTGAGTGGAGCGACAGTGCCCAGGGGTCTGAGTCTCACAGCTTGGGAAGCGGAGCTACTAACTTCA
GCCTGCTGAAGCAGGCTGGAGACGTGGAGGAGAACCCTGGACCTATGGAACTGGGACTGTCATGGATCTTCTTGCTGGCTATCCTG
AAGGGAGTGCAGTGTGAAGTTCAGCTGCTGCAGAGCGGACCCGAACTGGAAAAACCTGGCGCCTCCGTGATGATCAGCTGCAAGGC
CTCTGGCAGCTCCTTCACCGGCTACAACATGAACTGGGTCCGACAGAACATCGGCAAGAGCCTGGAATGGATCGGCGCCATCGATC
CTTACTACGGCGGCACCAGCTACAACCAGAAGTTCAAGGGCAGAGCCACACTGACCGTGGACAAGAGCAGCAGCACAGCCTACATG
CATCTGAAGTCCCTGACCAGCGAGGACAGCGCCGTGTACTACTGTGTGTCCGGCATGGAATACTGGGGCCAGGGCACAAGCGTGAC
AGTCTCTTCTGGCGGCGGTGGATCTGGCGGAGGCGGAAGTGGTGGCGGCGGATCTGATGTGGTCATGACACAGACCCCTCTGAGCC
TGCCTGTGTCTCTGGGAGATCAGGCCAGCATCAGCTGTAGAAGCAGCCAGAGCCTGGTGCACAGAAACGGCAACACCTACCTGCAC
TGGTATCTGCAGAAGCCCGGCCAGTCTCCTAAGCTGCTGATCCACAAGGTGTCCAACAGATTCAGCGGCGTGCCCGACAGATTCTC
TGGCTCTGGAAGCGGCACCGACTTCACCCTGAAGATTAGCAGAGTGGAAGCCGAGGACCTGGGCGTGTACTTCTGTAGCCAGAGCA
CACACGTGCCACCTCTGACATTTGGCGCTGGCACCAAGCTGGAACTGAAAAGGACCACGACGCCAGCGCCGCGACCACCAACACCG
GCGCCCACCATCGCGTCGCAGCCCCTGTCCCTGCGCCCAGAGGCGTGCCGGCCAGCGGCGGGGGGCGCAGTGCACACGAGGGGGCT
GGACTTCGCCTGTGATTTTTGGGTGCTGGTGGTGGTTGGTGGAGTCCTGGCTTGCTATAGCTTGCTAGTAACAGTGGCCTTTATTA
TTTTCTGGGTGAGGAGTAAGAGGAGCAGGCTCCTGCACAGTGACTACATGAACATGACTCCCCGCCGCCCCGGGCCCACCCGCAAG
CATTACCAGCCCTATGCCCCACCACGCGACTTCGCAGCCTATCGCTCCAAACGGGGCAGAAAGAAACTCCTGTATATATTCAAACA
ACCATTTATGAGACCAGTACAAACTACTCAAGAGGAAGATGGCTGTAGCTGCCGATTTCCAGAAGAAGAAGAAGGAGGATGTGAAC
TGAGAGTGAAGTTCAGCAGGAGCGCAGACGCCCCCGCGTACCAGCAGGGCCAGAACCAGCTCTATAACGAGCTCAATCTAGGACGA
AGAGAGGAGTACGATGTTTTGGACAAGAGACGTGGCCGGGACCCTGAGATGGGGGGAAAGCCGAGAAGGAAGAACCCTCAGGAAGG
CCTGTACAATGAACTGCAGAAAGATAAGATGGCGGAGGCCTACAGTGAGATTGGGATGAAAGGCGAGCGCCGGAGGGGCAAGGGGC
ACGATGGCCTTTACCAGGGTCTCAGTACAGCCACCAAGGACACCTACGACGCCCTTCACATGCAGGCCCTGCCCCCTCGCTAGCGG
TCTGGCTGTTCTAGAGGCTGGGTTTGGGAGGTGAAACCTAGTGGCAGGAGAACTTGTTTATTGCAGCTTATAATGGTTACAAATAA
AGCAATAGCATCACAAATTTCACAAATAAAGCATTTTTTTCACTGCATTCTAGTTGTGGTTTGTCCAAACTCATCAATGTATCTTA
TCATGTCTGGTAAGTCTTACATTCTTTTGTAAGCTGCTGAAAGTTGTGTATGAGTAGTCATATCATAAAGCTGCTTTGATATAAAA
AAGGTCTATGGCCATACTACCCTGAATGAGTCCCATCCCATCTGATATAAACAATCTGCATATTGGGATTGTCAGGGAATGTTCTT
AAAGATCAGATTAGTGGCACCTGCTGAGATACTGATGCACAGCATGGTTTCTGAACCAGTAGTTTCCCTGCAGTTGAGCAGGGAGC
AGCAGCAGCACTTGCACAAATACATATACACTCTTAACACTTCTTACCTACTGGCTTCCTCTAGCTTTTGTGGCAGCTTCAGGTAT
ATTTAGCACTGAACGAACATCTCAAGAAGGTATAGGCCTTTGTTTGTAAGTCCTGCTGTCCTAGCATCCTATAATCCTGGACTTCT
CCAGTACTTTCTGGCTGGATTGGTATCTGAGGCTAGTAGGAAGGGCTTGTTCCTGCTGGGTAGCTCTAAACAATGTATT
>FD4/6 (SEQ ID NO: 16)
TCTGCTAGAAAAAAAACAAAAAAGGCATGTATAGAGGAATTATGAGGGAAAGATACCAAGTCACGGTTTATTCTTCAAAATGGAGG
TGGCTTGTTGGGAAGGTGGAAGCTCATTTGGCCAGAGTGGAAATGGAATTGGGAGAAATCGATGACCAAATGTAAACACTTGGTGC
CTGATATAGCTTGACACCAAGTTAGCCCCAAGTGAAATACCCTGGCAATATTAATGTGTCTTTTCCCGATATTCCTCAGGTACTCC
AAAGATTCAGGTTTACTCACGTCATCCAGCAGAGAATGGAAAGTCAAATTTCCTGAATTGCTATGTGTCTGGGTTTCATCCATCCG
ACATTGAAGTTGACTTACTGAAGAATGGAGAGAGAATTGAAAAAGTGGAGCATTCAGACTTGTCTTTCAGCAAGGACTGGTCTTTC
TATCTCTTGTACTACACTGAATTCACCCCCACTGAAAAAGATGAGTATGCATGCCGCGTGAACCATGTGACTTTGTCACAGCCCAA
GATCGTGAAATGGGATCGAGACATGGGTGGAGGCGGTTCTGGAGGTGGCGGAAGTGGCGGCGGGGGAAGCGGTGGGGGCGGTTCCG
GCTCCCACTCCTTGAAGTATTTCCACACTTCCGTGTCCCGGCCCGGCCGCGGGGAGCCCCGCTTCATCTCTGTGGGCTACGTGGAC
GACACCCAGTTCGTGCGCTTCGACAACGACGCCGCGAGTCCGAGGATGGTGCCGCGGGCGCCGTGGATGGAGCAGGAGGGGTCAGA
GTATTGGGACCGGGAGACACGGAGCGCCAGGGACACCGCACAGATTTTCCGAGTGAATCTGCGGACGCTGCGCGGCTACTACAATC
AGAGCGAGGCCGGGTCTCACACCCTGCAGTGGATGCATGGCTGCGAGCTGGGGCCCGACGGGCGCTTCCTCCGCGGGTATGAACAG
TTCGCCTACGACGGCAAGGATTATCTCACCCTGAATGAGGACCTGCGCTCCTGGACCGCGGTGGACACGGCGGCTCAGATCTCCGA
GCAAAAGTCAAATGATGCCTCTGAGGCGGAGCACCAGAGAGCCTACCTGGAAGACACATGCGTGGAGTGGCTCCACAAATACCTGG
AGAAGGGGAAGGAGACGCTGCTTCACCTGGAGCCCCCAAAGACACACGTGACTCACCACCCCATCTCTGACCATGAGGCCACCCTG
AGGTGCTGGGCCCTGGGCTTCTACCCTGCGGAGATCACACTGACCTGGCAGCAGGATGGGGAGGGCCATACCCAGGACACGGAGCT
CGTGGAGACCAGGCCTGCAGGGGATGGAACCTTCCAGAAGTGGGCAGCTGTGGTGGTGCCTTCTGGAGAGGAGCAGAGATACACGT
GCCATGTGCAGCATGAGGGGCTACCCGAGCCCGTCACCCTGAGATGGAAGCCGGCTTCCCAGCCCACCATCCCCATCGTGGGCATC
ATTGCTGGCCTGGTTCTCCTTGGATCTGTGGTCTCTGGAGCTGTGGTTGCTGCTGTGATATGGAGGAAGAAGAGCTCAGGTGGAAA
AGGAGGGAGCTACTCTAAGGCTGAGTGGAGCGACAGTGCCCAGGGGTCTGAGTCTCACAGCTTGTAATCCGGTTATTTTCCACCAT
ATTGCCGTCTTTTGGCAATGTGAGGGCCCGGAAACCTGGCCCTGTCTTCTTGACGAGCATTCCTAGGGGTCTTTCCCCTCTCGCCA
AAGGAATGCAAGGTCTGTTGAATGTCGTGAAGGAAGCAGTTCCTCTGGAAGCTTCTTGAAGACAAACAACGTCTGTAGCGACCCTT
TGCAGGCAGCGGAACCCCCCACCTGGCGACAGGTGCCTCTGCGGCCAAAAGCCACGTGTATAAGATACACCTGCAAAGGCGGCACA
ACCCCAGTGCCACGTTGTGAGTTGGATAGTTGTGGAAAGAGTCAAATGGCTCTCCTCAAGCGTATTCAACAAGGGGCTGAAGGATG
CCCAGAAGGTACCCCATTGTATGGGATCTGATCTGGGGCCTCGGTGCACATGCTTTACATGTGTTTAGTCGAGGTTAAAAAACGTC
TAGGCCCCCCGAACCACGGGGACGTGGTTTTCCTTTGAAAAACACGATGATAATAGGATCCATGATGGAACTGGGACTGTCATGGA
TCTTCTTGCTGGCTATCCTGAAGGGAGTGCAGTGTGAAGTTCAGCTGCTGCAGAGCGGACCCGAACTGGAAAAACCTGGCGCCTCC
GTGATGATCAGCTGCAAGGCCTCTGGCAGCTCCTTCACCGGCTACAACATGAACTGGGTCCGACAGAACATCGGCAAGAGCCTGGA
ATGGATCGGCGCCATCGATCCTTACTACGGCGGCACCAGCTACAACCAGAAGTTCAAGGGCAGAGCCACACTGACCGTGGACAAGA
GCAGCAGCACAGCCTACATGCATCTGAAGTCCCTGACCAGCGAGGACAGCGCCGTGTACTACTGTGTGTCCGGCATGGAATACTGG
GGCCAGGGCACAAGCGTGACAGTCTCTTCTGGCGGCGGTGGATCTGGCGGAGGCGGAAGTGGTGGCGGCGGATCTGATGTGGTCAT
GACACAGACCCCTCTGAGCCTGCCTGTGTCTCTGGGAGATCAGGCCAGCATCAGCTGTAGAAGCAGCCAGAGCCTGGTGCACAGAA
ACGGCAACACCTACCTGCACTGGTATCTGCAGAAGCCCGGCCAGTCTCCTAAGCTGCTGATCCACAAGGTGTCCAACAGATTCAGC
GGCGTGCCCGACAGATTCTCTGGCTCTGGAAGCGGCACCGACTTCACCCTGAAGATTAGCAGAGTGGAAGCCGAGGACCTGGGCGT
GTACTTCTGTAGCCAGAGCACACACGTGCCACCTCTGACATTTGGCGCTGGCACCAAGCTGGAACTGAAAAGGACCACGACGCCAG
CGCCGCGACCACCAACACCGGCGCCCACCATCGCGTCGCAGCCCCTGTCCCTGCGCCCAGAGGCGTGCCGGCCAGCGGCGGGGGGC
GCAGTGCACACGAGGGGGCTGGACTTCGCCTGTGATTTTTGGGTGCTGGTGGTGGTTGGTGGAGTCCTGGCTTGCTATAGCTTGCT
AGTAACAGTGGCCTTTATTATTTTCTGGGTGAGGAGTAAGAGGAGCAGGCTCCTGCACAGTGACTACATGAACATGACTCCCCGCC
GCCCCGGGCCCACCCGCAAGCATTACCAGCCCTATGCCCCACCACGCGACTTCGCAGCCTATCGCTCCAAACGGGGCAGAAAGAAA
CTCCTGTATATATTCAAACAACCATTTATGAGACCAGTACAAACTACTCAAGAGGAAGATGGCTGTAGCTGCCGATTTCCAGAAGA
AGAAGAAGGAGGATGTGAACTGAGAGTGAAGTTCAGCAGGAGCGCAGACGCCCCCGCGTACCAGCAGGGCCAGAACCAGCTCTATA
ACGAGCTCAATCTAGGACGAAGAGAGGAGTACGATGTTTTGGACAAGAGACGTGGCCGGGACCCTGAGATGGGGGGAAAGCCGAGA
AGGAAGAACCCTCAGGAAGGCCTGTACAATGAACTGCAGAAAGATAAGATGGCGGAGGCCTACAGTGAGATTGGGATGAAAGGCGA
GCGCCGGAGGGGCAAGGGGCACGATGGCCTTTACCAGGGTCTCAGTACAGCCACCAAGGACACCTACGACGCCCTTCACATGCAGG
CCCTGCCCCCTCGCTAGCGGTCTGGCTGTTCTAGAGGCTGGGTTTGGGAGGTGAAACCTAGTGGCAGGAGAACTTGTTTATTGCAG
CTTATAATGGTTACAAATAAAGCAATAGCATCACAAATTTCACAAATAAAGCATTTTTTTCACTGCATTCTAGTTGTGGTTTGTCC
AAACTCATCAATGTATCTTATCATGTCTGGTAAGTCTTACATTCTTTTGTAAGCTGCTGAAAGTTGTGTATGAGTAGTCATATCAT
AAAGCTGCTTTGATATAAAAAAGGTCTATGGCCATACTACCCTGAATGAGTCCCATCCCATCTGATATAAACAATCTGCATATTGG
GATTGTCAGGGAATGTTCTTAAAGATCAGATTAGTGGCACCTGCTGAGATACTGATGCACAGCATGGTTTCTGAACCAGTAGTTTC
CCTGCAGTTGAGCAGGGAGCAGCAGCAGCACTTGCACAAATACATATACACTCTTAACACTTCTTACCTACTGGCTTCCTCTAGCT
TTTGTGGCAGCTTCAGGTATATTTAGCACTGAACGAACATCTCAAGAAGGTATAGGCCTTTGTTTGTAAGTCCTGCTGTCCTAGCA
TCCTATAATCCTGGACTTCTCCAGTACTTTCTGGCTGGATTGGTATCTGAGGCTAGTAGGAAGGGCTTGTTCCTGCTGGGTAGCTC
TAAACAATGTATT
>FD7 (SEQ ID NO: 17)
TCTGCATATTGGGATTGTCAGGGAATGTTCTTAAAGATCAGATTAGTGGCACCTGCTGAGATACTGATGCACAGCATGGTTTCTGA
ACCAGTAGTTTCCCTGCAGTTGAGCAGGGAGCAGCAGCAGCACTTGCACAAATACATATACACTCTTAACACTTCTTACCTACTGG
CTTCCTCTAGCTTTTGTGGCAGCTTCAGGTATATTTAGCACTGAACGAACATCTCAAGAAGGTATAGGCCTTTGTTTGTAAGTCCT
GCTGTCCTAGCATCCTATAATCCTGGACTTCTCCAGTACTTTCTGGCTGGATTGGTATCTGAGGCTAGTAGGAAGGGCTTGTTCCT
GCTGGGTAGCTCTAAACAATGTATTCATGGGTAGGAACAGCAGCCTATTCTGCCAGCCTTATTTCTAACCATTTTAGACATTTGTT
AGTACATGGTATTTTAAAAGTAAAACTTAATGTCTTCCTTTTTTTTCTCCACTGTCTTTTTCATAGATCGAGACATGGGTGGAGGC
GGTTCTGGAGGTGGCGGAAGTGGCGGCGGGGGAAGCGGTGGGGGCGGTTCCGGCTCCCACTCCTTGAAGTATTTCCACACTTCCGT
GTCCCGGCCCGGCCGCGGGGAGCCCCGCTTCATCTCTGTGGGCTACGTGGACGACACCCAGTTCGTGCGCTTCGACAACGACGCCG
CGAGTCCGAGGATGGTGCCGCGGGCGCCGTGGATGGAGCAGGAGGGGTCAGAGTATTGGGACCGGGAGACACGGAGCGCCAGGGAC
ACCGCACAGATTTTCCGAGTGAATCTGCGGACGCTGCGCGGCTACTACAATCAGAGCGAGGCCGGGTCTCACACCCTGCAGTGGAT
GCATGGCTGCGAGCTGGGGCCCGACGGGCGCTTCCTCCGCGGGTATGAACAGTTCGCCTACGACGGCAAGGATTATCTCACCCTGA
ATGAGGACCTGCGCTCCTGGACCGCGGTGGACACGGCGGCTCAGATCTCCGAGCAAAAGTCAAATGATGCCTCTGAGGCGGAGCAC
CAGAGAGCCTACCTGGAAGACACATGCGTGGAGTGGCTCCACAAATACCTGGAGAAGGGGAAGGAGACGCTGCTTCACCTGGAGCC
CCCAAAGACACACGTGACTCACCACCCCATCTCTGACCATGAGGCCACCCTGAGGTGCTGGGCCCTGGGCTTCTACCCTGCGGAGA
TCACACTGACCTGGCAGCAGGATGGGGAGGGCCATACCCAGGACACGGAGCTCGTGGAGACCAGGCCTGCAGGGGATGGAACCTTC
CAGAAGTGGGCAGCTGTGGTGGTGCCTTCTGGAGAGGAGCAGAGATACACGTGCCATGTGCAGCATGAGGGGCTACCCGAGCCCGT
CACCCTGAGATGGAAGCCGGCTTCCCAGCCCACCATCCCCATCGTGGGCATCATTGCTGGCCTGGTTCTCCTTGGATCTGTGGTCT
CTGGAGCTGTGGTTGCTGCTGTGATATGGAGGAAGAAGAGCTCAGGTGGAAAAGGAGGGAGCTACTCTAAGGCTGAGTGGAGCGAC
AGTGCCCAGGGGTCTGAGTCTCACAGCTTGGGAAGCGGAGCTACTAACTTCAGCCTGCTGAAGCAGGCTGGAGACGTGGAGGAGAA
CCCTGGACCTATGGAACTGGGACTGTCATGGATCTTCTTGCTGGCTATCCTGAAGGGAGTGCAGTGTGAAGTTCAGCTGCTGCAGA
GCGGACCCGAACTGGAAAAACCTGGCGCCTCCGTGATGATCAGCTGCAAGGCCTCTGGCAGCTCCTTCACCGGCTACAACATGAAC
TGGGTCCGACAGAACATCGGCAAGAGCCTGGAATGGATCGGCGCCATCGATCCTTACTACGGCGGCACCAGCTACAACCAGAAGTT
CAAGGGCAGAGCCACACTGACCGTGGACAAGAGCAGCAGCACAGCCTACATGCATCTGAAGTCCCTGACCAGCGAGGACAGCGCCG
TGTACTACTGTGTGTCCGGCATGGAATACTGGGGCCAGGGCACAAGCGTGACAGTCTCTTCTGGCGGCGGTGGATCTGGCGGAGGC
GGAAGTGGTGGCGGCGGATCTGATGTGGTCATGACACAGACCCCTCTGAGCCTGCCTGTGTCTCTGGGAGATCAGGCCAGCATCAG
CTGTAGAAGCAGCCAGAGCCTGGTGCACAGAAACGGCAACACCTACCTGCACTGGTATCTGCAGAAGCCCGGCCAGTCTCCTAAGC
TGCTGATCCACAAGGTGTCCAACAGATTCAGCGGCGTGCCCGACAGATTCTCTGGCTCTGGAAGCGGCACCGACTTCACCCTGAAG
ATTAGCAGAGTGGAAGCCGAGGACCTGGGCGTGTACTTCTGTAGCCAGAGCACACACGTGCCACCTCTGACATTTGGCGCTGGCAC
CAAGCTGGAACTGAAAAGGACCACGACGCCAGCGCCGCGACCACCAACACCGGCGCCCACCATCGCGTCGCAGCCCCTGTCCCTGC
GCCCAGAGGCGTGCCGGCCAGCGGCGGGGGGCGCAGTGCACACGAGGGGGCTGGACTTCGCCTGTGATTTTTGGGTGCTGGTGGTG
GTTGGTGGAGTCCTGGCTTGCTATAGCTTGCTAGTAACAGTGGCCTTTATTATTTTCTGGGTGAGGAGTAAGAGGAGCAGGCTCCT
GCACAGTGACTACATGAACATGACTCCCCGCCGCCCCGGGCCCACCCGCAAGCATTACCAGCCCTATGCCCCACCACGCGACTTCG
CAGCCTATCGCTCCAAACGGGGCAGAAAGAAACTCCTGTATATATTCAAACAACCATTTATGAGACCAGTACAAACTACTCAAGAG
GAAGATGGCTGTAGCTGCCGATTTCCAGAAGAAGAAGAAGGAGGATGTGAACTGAGAGTGAAGTTCAGCAGGAGCGCAGACGCCCC
CGCGTACCAGCAGGGCCAGAACCAGCTCTATAACGAGCTCAATCTAGGACGAAGAGAGGAGTACGATGTTTTGGACAAGAGACGTG
GCCGGGACCCTGAGATGGGGGGAAAGCCGAGAAGGAAGAACCCTCAGGAAGGCCTGTACAATGAACTGCAGAAAGATAAGATGGCG
GAGGCCTACAGTGAGATTGGGATGAAAGGCGAGCGCCGGAGGGGCAAGGGGCACGATGGCCTTTACCAGGGTCTCAGTACAGCCAC
CAAGGACACCTACGACGCCCTTCACATGCAGGCCCTGCCCCCTCGCTAGCTGCAGGGTCTGGCTGTTCTAGAGGCTGGGTTTGGGA
GGTGAAACCTAGTGGCAGGAGAACTTGTTTATTGCAGCTTATAATGGTTACAAATAAAGCAATAGCATCACAAATTTCACAAATAA
AGCATTTTTTTCACTGCATTCTAGTTGTGGTTTGTCCAAACTCATCAATGTATCTTATCATGTCTGAGGTAAGTTTTTGACCTTGA
GAAAATGTTTTTGTTTCACTGTCCTGAGGACTATTTATAGACAGCTCTAACATGATAACCCTCACTATGTGGAGAACATTGACAGA
GTAACATTTTAGCAGGGAAAGAAGAATCCTACAGGGTCATGTTCCCTTCTCCTGTGGAGTGGCATGAAGAAGGTGTATGGCCCCAG
GTATGGCCATATTACTGACCCTCTACAGAGAGGGCAAAGGAACTGCCAGTATGGTATTGCAGGATAAAGGCAGGTGGTTACCCACA
TTACCTGCAAGGCTTTGATCTTTCTTCTGCCATTTCCACATTGGACATCTCTGCTGAGGAGAGAAAATGAACCACTCTTTTCCTTT
GTATAATGTTGTTTTATTCTTCAGACAGAAGAGAGGAGTTATACAGCTCTGCAGACATCCCATTCCTGTATGGGGACTGTGTTTGC
CTCTTAGAGGTTCCCAGGCCACTAGAGGAGATAAAGGGAAACAGATTGTT
>FD8 (SEQ ID NO: 18)
TCTGCATATTGGGATTGTCAGGGAATGTTCTTAAAGATCAGATTAGTGGCACCTGCTGAGATACTGATGCACAGCATGGTTTCTGA
ACCAGTAGTTTCCCTGCAGTTGAGCAGGGAGCAGCAGCAGCACTTGCACAAATACATATACACTCTTAACACTTCTTACCTACTGG
CTTCCTCTAGCTTTTGTGGCAGCTTCAGGTATATTTAGCACTGAACGAACATCTCAAGAAGGTATAGGCCTTTGTTTGTAAGTCCT
GCTGTCCTAGCATCCTATAATCCTGGACTTCTCCAGTACTTTCTGGCTGGATTGGTATCTGAGGCTAGTAGGAAGGGCTTGTTCCT
GCTGGGTAGCTCTAAACAATGTATTCATGGGTAGGAACAGCAGCCTATTCTGCCAGCCTTATTTCTAACCATTTTAGACATTTGTT
AGTACATGGTATTTTAAAAGTAAAACTTAATGTCTTCCTTTTTTTTCTCCACTGTCTTTTTCATAGATCGAGACATGGGTGGAGGC
GGTTCTGGAGGTGGCGGAAGTGGCGGCGGGGGAAGCGGTGGGGGCGGTTCCGGCTCCCACTCCTTGAAGTATTTCCACACTTCCGT
GTCCCGGCCCGGCCGCGGGGAGCCCCGCTTCATCTCTGTGGGCTACGTGGACGACACCCAGTTCGTGCGCTTCGACAACGACGCCG
CGAGTCCGAGGATGGTGCCGCGGGCGCCGTGGATGGAGCAGGAGGGGTCAGAGTATTGGGACCGGGAGACACGGAGCGCCAGGGAC
ACCGCACAGATTTTCCGAGTGAATCTGCGGACGCTGCGCGGCTACTACAATCAGAGCGAGGCCGGGTCTCACACCCTGCAGTGGAT
GCATGGCTGCGAGCTGGGGCCCGACGGGCGCTTCCTCCGCGGGTATGAACAGTTCGCCTACGACGGCAAGGATTATCTCACCCTGA
ATGAGGACCTGCGCTCCTGGACCGCGGTGGACACGGCGGCTCAGATCTCCGAGCAAAAGTCAAATGATGCCTCTGAGGCGGAGCAC
CAGAGAGCCTACCTGGAAGACACATGCGTGGAGTGGCTCCACAAATACCTGGAGAAGGGGAAGGAGACGCTGCTTCACCTGGAGCC
CCCAAAGACACACGTGACTCACCACCCCATCTCTGACCATGAGGCCACCCTGAGGTGCTGGGCCCTGGGCTTCTACCCTGCGGAGA
TCACACTGACCTGGCAGCAGGATGGGGAGGGCCATACCCAGGACACGGAGCTCGTGGAGACCAGGCCTGCAGGGGATGGAACCTTC
CAGAAGTGGGCAGCTGTGGTGGTGCCTTCTGGAGAGGAGCAGAGATACACGTGCCATGTGCAGCATGAGGGGCTACCCGAGCCCGT
CACCCTGAGATGGAAGCCGGCTTCCCAGCCCACCATCCCCATCGTGGGCATCATTGCTGGCCTGGTTCTCCTTGGATCTGTGGTCT
CTGGAGCTGTGGTTGCTGCTGTGATATGGAGGAAGAAGAGCTCAGGTGGAAAAGGAGGGAGCTACTCTAAGGCTGAGTGGAGCGAC
AGTGCCCAGGGGTCTGAGTCTCACAGCTTGGGAAGCGGAGCTACTAACTTCAGCCTGCTGAAGCAGGCTGGAGACGTGGAGGAGAA
CCCTGGACCTATGGAACTGGGACTGTCATGGATCTTCTTGCTGGCTATCCTGAAGGGAGTGCAGTGTGAAGTTCAGCTGCTGCAGA
GCGGACCCGAACTGGAAAAACCTGGCGCCTCCGTGATGATCAGCTGCAAGGCCTCTGGCAGCTCCTTCACCGGCTACAACATGAAC
TGGGTCCGACAGAACATCGGCAAGAGCCTGGAATGGATCGGCGCCATCGATCCTTACTACGGCGGCACCAGCTACAACCAGAAGTT
CAAGGGCAGAGCCACACTGACCGTGGACAAGAGCAGCAGCACAGCCTACATGCATCTGAAGTCCCTGACCAGCGAGGACAGCGCCG
TGTACTACTGTGTGTCCGGCATGGAATACTGGGGCCAGGGCACAAGCGTGACAGTCTCTTCTGGCGGCGGTGGATCTGGCGGAGGC
GGAAGTGGTGGCGGCGGATCTGATGTGGTCATGACACAGACCCCTCTGAGCCTGCCTGTGTCTCTGGGAGATCAGGCCAGCATCAG
CTGTAGAAGCAGCCAGAGCCTGGTGCACAGAAACGGCAACACCTACCTGCACTGGTATCTGCAGAAGCCCGGCCAGTCTCCTAAGC
TGCTGATCCACAAGGTGTCCAACAGATTCAGCGGCGTGCCCGACAGATTCTCTGGCTCTGGAAGCGGCACCGACTTCACCCTGAAG
ATTAGCAGAGTGGAAGCCGAGGACCTGGGCGTGTACTTCTGTAGCCAGAGCACACACGTGCCACCTCTGACATTTGGCGCTGGCAC
CAAGCTGGAACTGAAAAGGACCACGACGCCAGCGCCGCGACCACCAACACCGGCGCCCACCATCGCGTCGCAGCCCCTGTCCCTGC
GCCCAGAGGCGTGCCGGCCAGCGGGGGGGGCGCAGTGCACACGAGGGGGCTGGACTTCGCCTGTGATTTTTGGGTGCTGGTGGTG
GTTGGTGGAGTCCTGGCTTGCTATAGCTTGCTAGTAACAGTGGCCTTTATTATTTTCTGGGTGAGGAGTAAGAGGAGCAGGCTCCT
GCACAGTGACTACATGAACATGACTCCCCGCCGCCCCGGGCCCACCCGCAAGCATTACCAGCCCTATGCCCCACCACGCGACTTCG
CAGCCTATCGCTCCAAACGGGGCAGAAAGAAACTCCTGTATATATTCAAACAACCATTTATGAGACCAGTACAAACTACTCAAGAG
GAAGATGGCTGTAGCTGCCGATTTCCAGAAGAAGAAGAAGGAGGATGTGAACTGAGAGTGAAGTTCAGCAGGAGCGCAGACGCCCC
CGCGTACCAGCAGGGCCAGAACCAGCTCTATAACGAGCTCAATCTAGGACGAAGAGAGGAGTACGATGTTTTGGACAAGAGACGTG
GCCGGGACCCTGAGATGGGGGGAAAGCCGAGAAGGAAGAACCCTCAGGAAGGCCTGTACAATGAACTGCAGAAAGATAAGATGGCG
GAGGCCTACAGTGAGATTGGGATGAAAGGCGAGCGCCGGAGGGGCAAGGGGCACGATGGCCTTTACCAGGGTCTCAGTACAGCCAC
CAAGGACACCTACGACGCCCTTCACATGCAGGCCCTGCCCCCTCGCTAGCTGCAGGGTCTGGCTGTTCTAGAGGCTGGGTTTGGGA
GGTGAAACCTAGTGGCAGGAGAACTTGTTTATTGCAGCTTATAATGGTTACAAATAAAGCAATAGCATCACAAATTTCACAAATAA
AGCATTTTTTTCACTGCATTCTAGTTGTGGTTTGTCCAAACTCATCAATGTATCTTATCATGTCTGAGGTAAGTTTTTGACCTTGA
GAAAATGTTTTTGTTTCACTGTCCTGAGGACTATTTATAGACAGCTCTAACATGATAACCCTCACTATGTGGAGAACATTGACAGA
GTAACATTTTAGCAGGGAAAGAAGAATCCTACAGGGTCATGTTCCCTTCTCCTGTGGAGTGGCATGAAGAAGGTGTATGGCCCCAG
GTATGGCCATATTACTGACCCTCTACAGAGAGGGCAAAGGAACTGCCAGTATGGTATTGCAGGATAAAGGCAGGTGGTTACCCACA
TTACCTGCAAGGCTTTGATCTTTCTTCTGCCATTTCCACATTGGACATCTCTGCTGAGGAGAGAAAATGAACCACTCTTTTCCTTT
GTATAATGTTGTTTTATTCTTCAGACAGAAGAGAGGAGTTATACAGCTCTGCAGACATCCCATTCCTGTATGGGGACTGTGTTTGC
CTCTTAGAGGTTCCCAGGCCACTAGAGGAGATAAAGGGAAACAGATTGTT

### Example 5

### Expression of HLA-E on Cells Lacking HLA-A/B/C

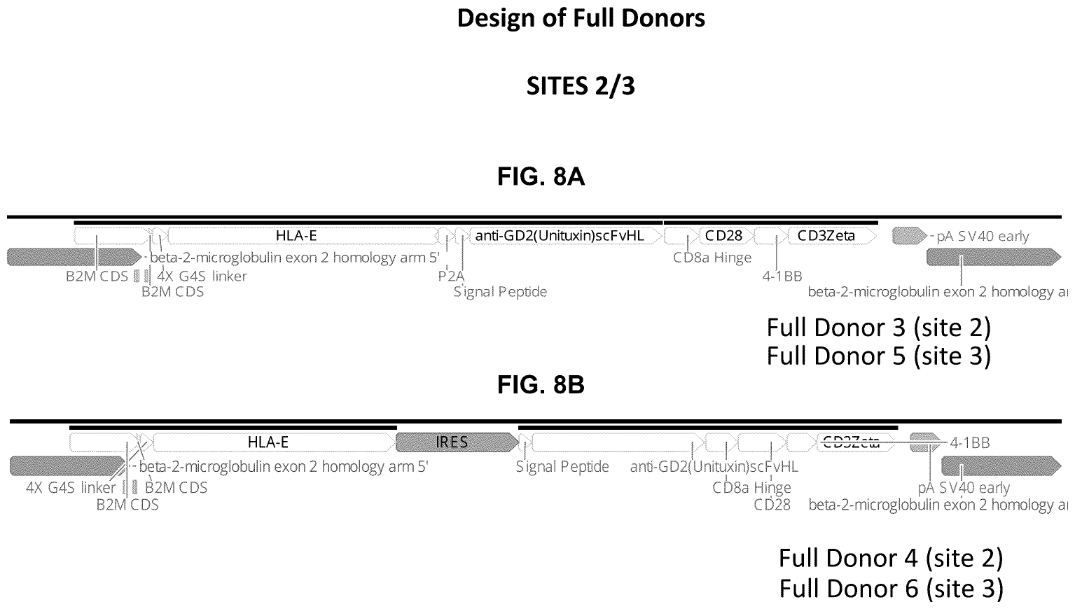

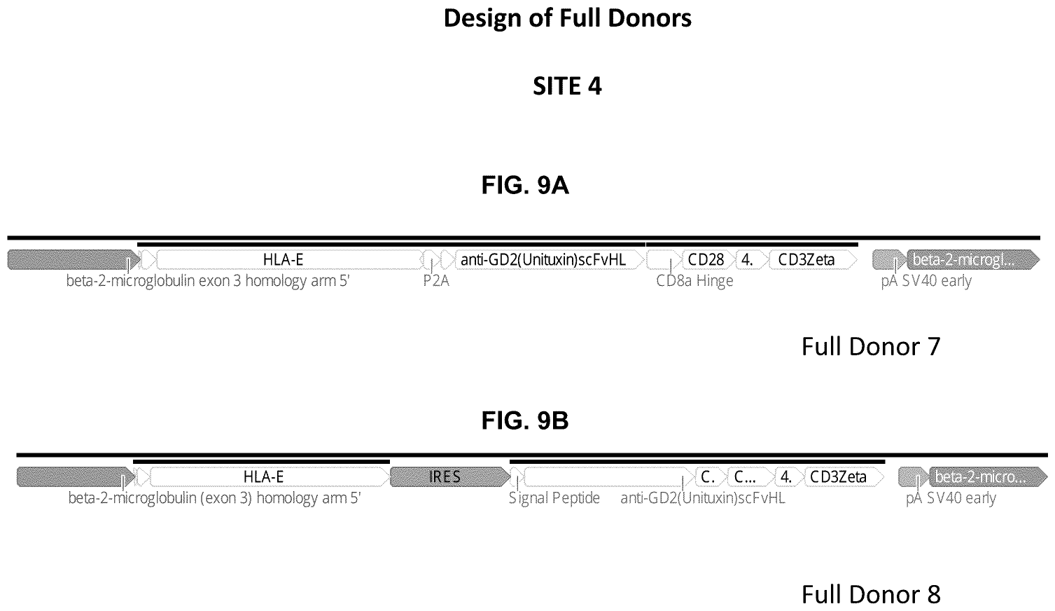

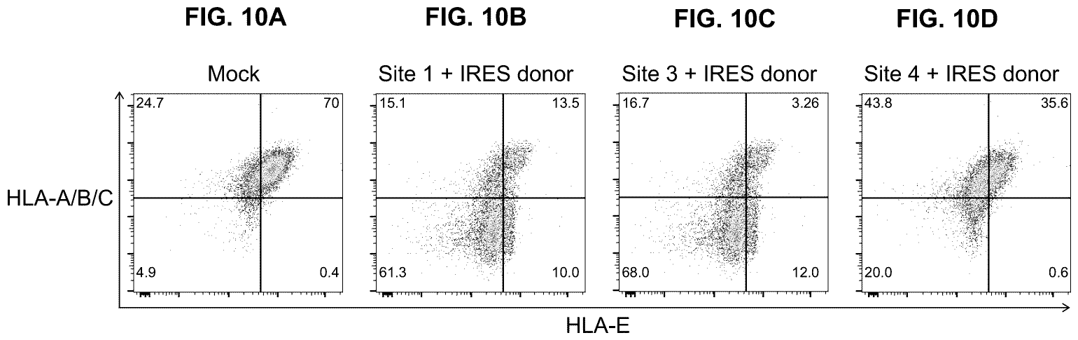

To determine the HLA-E expression induced by the bicistronic constructs comprising a CAR component and an ISMM component connected by an IRES, primary isolated CD4+ T-cells were transduced with 1×10⁶ AAV6 genomes per cell carrying the full donor constructs targeting Sites 1, 3 or 4 on B2M, and HLA-E one day before electroporation using an Amaxa 4D nucleofector and P3 solution with purified Cas 9 protein preloaded with CRISPR guide RNAs targeting the sites of SEQ ID NO: 1, 3, or 4. Expression was determined using flow cytometry analyzing loss of HLA-A/B/C expression and while retaining HLA-E expression. The constructs tested were Full Donor 2 (FIG. 7B), Full Donor 6 (FIG. 8B) and Full Donor 8 (FIG. 9B). The highest levels of HLA-E expression were observed for bicistronic constructs directed to Site 1 and Site 3. See FIG. 10A-10D.

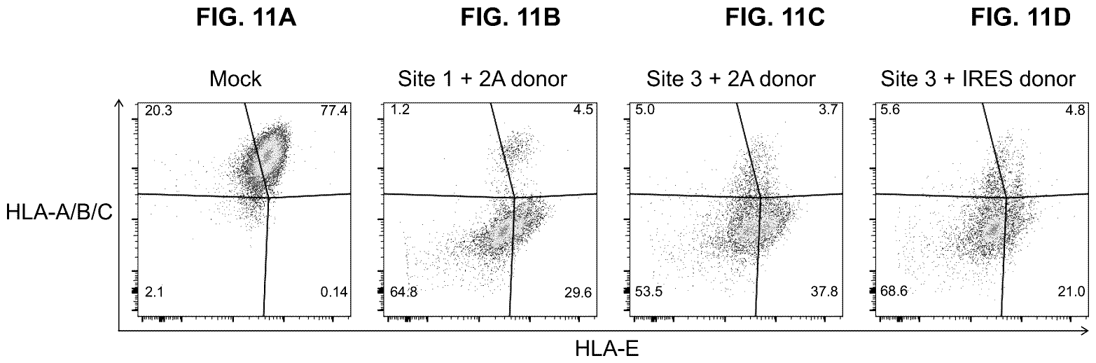

Similarly, HLA-E expression induced by the bicistronic constructs comprising a CAR component and an ISMM component connected by a 2A element was studied. T-cells were transduced with 1×10⁶ AAV6 genomes per cell carrying the full donor constructs targeting Sites 1 or 3 on B2M, and HLA-E one day before electroporation using an Amaxa 4D nucleofector and P3 solution with purified Cas 9 protein preloaded with CRISPR guide RNAs targeting the sites of SEQ ID NO: 1 or 3. Expression was determined using flow cytometry analyzing loss of HLA-A/B/C expression and while retaining HLA-E expression. The constructs tested were Full Donor 1 (FIG. 7A), Full Donor 5 (FIG. 8A) and Full Donor 6 (FIG. 8B). Highest levels of HLA-E expression were observed consistently for bicistronic constructs with the CAR and ISMM components connected by a 2A element (P2A) compared to the results obtained when the two components were connected via an IRES element. See FIG. 11A-11D.

### Example 6

### Anti-Tumor Efficacy of Anti-GD2 CAR Engineered Cells

To address the killing efficacy of engineered CAR-T cells expressing bicistronic constructs comprising an anti-GD2 CAR having an scFv derived from UNITUXIN® and an ISMM comprising a truncated B2M and HLA-E, the engineered T-cells were co-cultured with GD2+CHP134 neuroblastoma cells expressing a mKate2 fluorescent cell tracker at different ratios (1:1, 2:1, 4:1, 10:1). Populations of mKate2+ cells were determined over a time course of 24 hours with images taken every hour in a Sartorius Incucyte S3 live cell imaging system. Cells numbers were normalized to the number of mKate2+ cells at the starting timepoint.

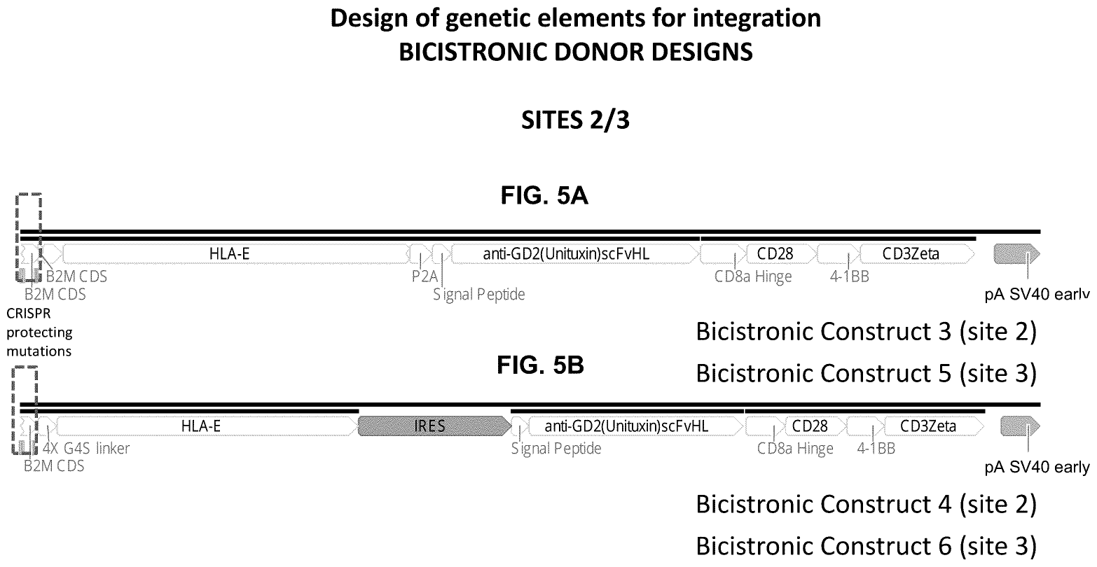

The different bicistronic constructs were used: “Site 1 2A CAR-HLAE” (corresponding to Bicistronic Construct 1 of FIG. 4A and Full Donor 1 of FIG. 7A), “Site 3 2A CAR-HLAE” (corresponding to Bicistronic Construct 5 of FIG. 5A and Full Donor 5 of FIG. 8A), and “Site 3 IRES CAR-HLAE” (corresponding to Bicistronic Construct 6 of FIG. 5B and Full Donor 6 of FIG. 8B).

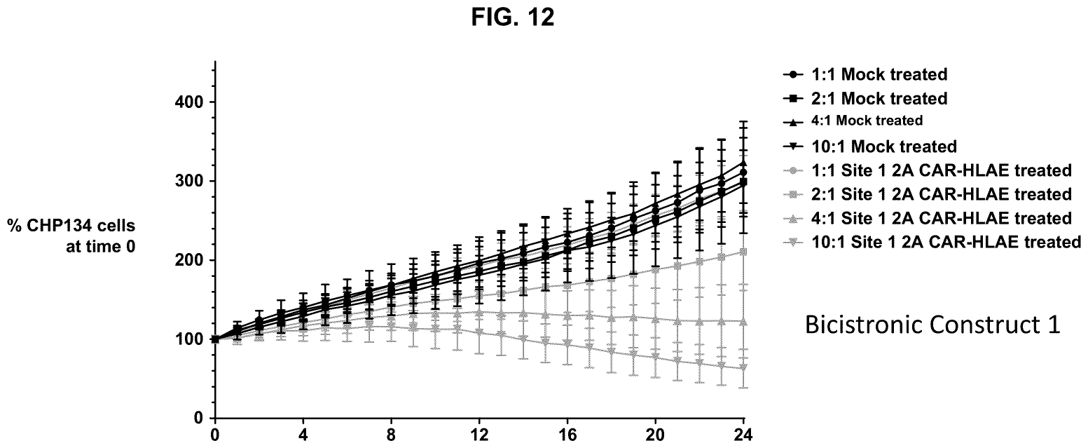

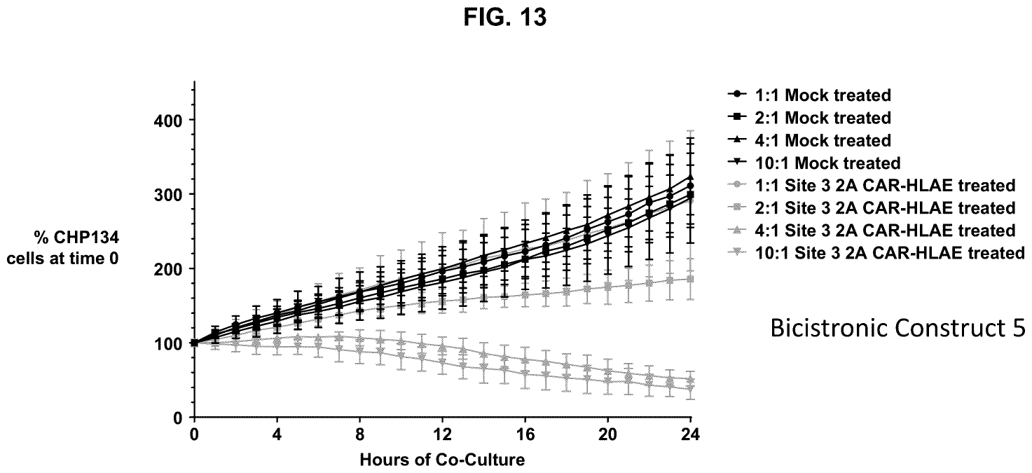

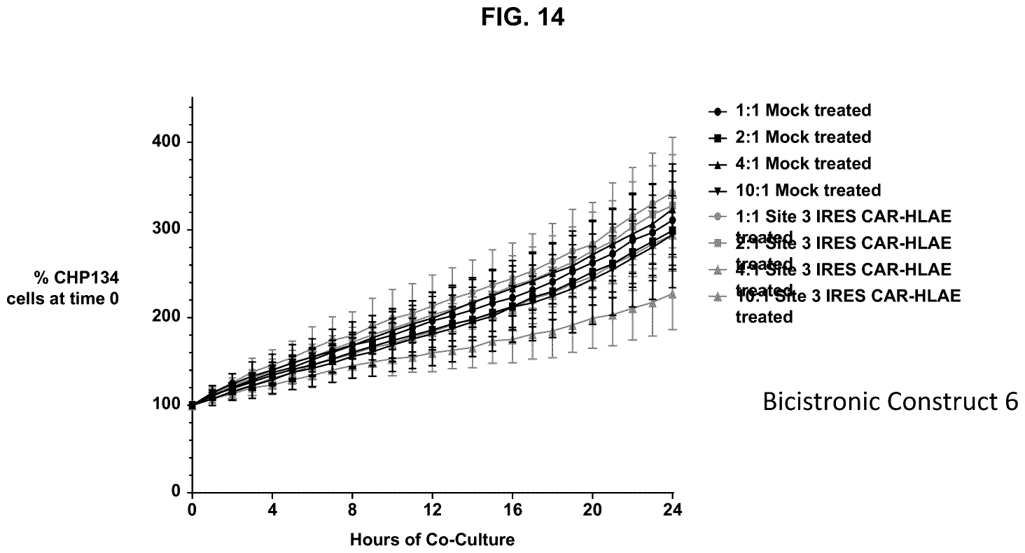

Co-culture of neuroblastoma cells with engineered CAR-T cells expressing the Site 3 IRES CAR-HLAE bicistronic construct showed negligible effects except at the highest CAR-T cell to neuroblastoma cell ratio (10:1). See FIG. 14. Significantly better results were obtained when the bicistronic constructs comprised a P2A element between the CAR and ISMM elements. See FIG. 12 and FIG. 13.

Decreases in neuroblastoma cells with respect to control conditions were observed at the 2:1, 4:1 and 10:1 ratios when using the Site 1 2A CAR-HLAE bicistronic construct. However, despite the reductions in cell growth with respect to control conditions, a reduction in total number of cells with respect to the initial cell number was only achieved at the highest ratio (10:1). See FIG. 12.

When the Site 3 2A CAR-HLAE bicistronic construct was used, a reduction in the number of neuroblastoma cells with respect to control conditions was observed at 2:1, 4:1 and 10:1 ratios. Reductions in the total numbers of cells with respect to the initial cell number were observed at the 4:1 and 10:1 ratios. At a 4:1 ratio, the observed effects were similar to those observed at the 10:1 ratio for the Site 1 2A CAR-HLAE bicistronic construct. At the 10:1 ratio, the reduction in cell growth observed for Site 3 2A CAR-HLAE was higher than for Site 1 2A CAR-HLAE at the same ratio. See FIG. 13.

In summary, the most pronounced effects were observed for constructs that comprised a P2A element between the CAR and ISMM components of the bicistronic construct instead of an IRES element, and bicistronic construct with a P2A element targeting insertion Site 3 of B2M were more effective than those targeting Site 1.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described herein.

All publications, patent applications, patents, and other references mentioned herein are incorporated by reference in their entirety. Database entries and electronic publications disclosed in the present disclosure are incorporated by reference in their entireties. The version of the database entry or electronic publication incorporated by reference in the present application is the most recent version of the database entry or electronic publication that was publicly available at the time the present application was filed. The database entries corresponding to gene or protein identifiers (e.g., genes or proteins identified by an accession number or database identifier of a public database such as Genbank, Refseq, or Uniprot) disclosed in the present application are incorporated by reference in their entireties. The gene or protein-related incorporated information is not limited to the sequence data contained in the database entry. The information incorporated by reference includes the entire contents of the database entry in the most recent version of the database that was publicly available at the time the present application was filed. In case of conflict, the present specification, including definitions, will control. In addition, the materials, methods, and examples are illustrative only and not intended to be limiting.

1. A bicistronic polynucleotide encoding
(i) a therapeutic agent; and,
(ii) an immune surveillance masking molecule (ISMM), wherein the ISMM comprises a non-functional beta-2-microglobulin (B2M) polypeptide and a human leukocyte antigen (HLA).

2. The bicistronic polynucleotide of claim 1, wherein the therapeutic agent is a chimeric antigen receptor (CAR) comprising an antigen-binding domain of an antibody that specifically binds to disialoganglioside GD2.

3-4. (canceled)

5. The bicistronic polynucleotide of claim 2, wherein the antigen-binding domain of the CAR comprises:
(i) dinutuximab or an antigen-binding portion thereof; or,
(ii) a single-chain variable fragment (scFv) comprising the variable region of the heavy chain (VH) and the variable region of the light chain (VL) of dinutuximab; or,
(iii) a dinutuximab scFv comprising the protein sequence set forth in SEQ ID NO: 22; or,
(iv) an antigen-binding domain which cross-competes with dinutuximab; or,
(v) an antigen-binding domain which binds to the same epitope as dinutuximab; or,
(vi) an antigen-binding domain comprising the VH CDR3 of dinutuximab, and optionally the VH CDR1 and a VH CDR2 of dinutuximab; or,
(vii) a VH CDR1 comprising the VH CDR1 of dinutuximab and/or a VH CDR2 comprising the VH CDR2 of dinutuximab; or,
(viii) a VL CDR1 of dinutuximab, a VL CDR2 of dinutuximab, and/or a VL CDR3 of dinutuximab; or,
(ix) a VL CDR1 comprising the VL CDR1 of dinutuximab, a VL CDR2 comprising the VL CDR2 of dinutuximab, and/or a VL CDR3 comprising the VL CDR3 of dinutuximab; or,
(x) a VH CDR1 of SEQ ID NO: 59; a VH CDR2 of SEQ ID NO: 63; and a VH CDR3 of SEQ ID NO: 67; and/or a VL CDR1 of SEQ ID NO: 71; a VL CDR2 of SEQ ID NO: 75; and a VL CDR3 of SEQ ID NO: 79; or,
(xi) a VH CDR1 of SEQ ID NO: 60; a VH CDR2 of SEQ ID NO: 64; and a VH CDR3 of SEQ ID NO: 68; and/or a VL CDR1 of SEQ ID NO: 72; a VL CDR2 of SEQ ID NO: 76; and a VL CDR3 of SEQ ID NO: 80; or,
(xii) a VH CDR1 of SEQ ID NO: 61; a VH CDR2 of SEQ ID NO: 65; and a VH CDR3 of SEQ ID NO: 69; and/or a VL CDR1 of SEQ ID NO: 73; a VL CDR2 of SEQ ID NO: 77; and a VL CDR3 of SEQ ID NO: 81; or,
(xiii) a VH CDR1 of SEQ ID NO: 62; a VH CDR2 of SEQ ID NO: 66; and a VH CDR3 of SEQ ID NO: 70; and/or a VL CDR1 of SEQ ID NO: 74; a VL CDR2 of SEQ ID NO: 78; and a VL CDR3 of SEQ ID NO: 82; or,
(xiv) a VH CDR1 of SEQ ID NO: 53; a VH CDR2 of SEQ ID NO: 54; and a VH CDR3 of SEQ ID NO: 55; and/or a VL CDR1 of SEQ ID NO: 56; a VL CDR2 of SEQ ID NO: 57; and a VL CDR3 of SEQ ID NO: 58; or,
(xv) a VH and a VL, wherein the VH comprises the protein sequence set forth in SEQ ID NO: 44 or the VL comprises the protein sequence set forth in SEQ ID NO: 46; or,
(xvi) a VH comprising the protein sequence set forth in SEQ ID NO:44 and a VL comprising the protein sequence set forth in SEQ ID NO: 46.

6-21. (canceled)

22. The bicistronic polynucleotide of claim 2, wherein the CAR is
(i) a standard CAR, a split CAR, an off-switch CAR, an on-switch CAR, a first-generation CAR, a second-generation CAR, a third-generation CAR, or a fourth-generation CAR; and,
(ii) the antigen-binding domain is an lg NAR, a Fab, a Fab′, a F(ab)′2, a F(ab)′3, an Fv, a single chain variable fragment (scFv), a bis-scFv, a (scFv)2, a minibody, a diabody, a triabody, a tetrabody, an intrabody, a disulfide stabilized Fv protein (dsFv), a unibody, a nanobody, an affibody, a DARPin, a monobody, an adnectin, an alphabody, or a designed binder.

23. (canceled)

24. The bicistronic polynucleotide of claim 5, wherein the CAR further comprises
(i) a transmembrane domain;
(ii) an intracellular domain comprising a signaling domain derived from CD3zeta, FcR gamma, FcR beta, CD3 gamma, CD3 delta, CD3 epsilon, CD5, CD22, CD79a, CD79b, CD66d, or CD28;
(iii) a spacer located between the antigen-binding domain and the transmembrane domain; and optionally,
(iv) a co-stimulatory domain derived from 2B4, HVEM, ICOS, LAG3, DAP10, DAP12, CD27, CD28, 4-1BB (CD137), OX40 (CD134), CD30, CD40, ICOS (CD278), glucocorticoid-induced tumor necrosis factor receptor (GITR), lymphocyte function-associated antigen-1 (LFA-1), CD2, CD7, LIGHT, NKG2C, B7-H3, CD3zeta, and combinations thereof.

25-36. (canceled)

37. The bicistronic polynucleotide of claim 2, wherein
(i) the polynucleotide encoding the CAR comprises the nucleic acid sequence set forth in SEQ ID NO: 19; and/or,
(ii) the polynucleotide encodes the CAR protein set forth in SEQ ID NO: 20.

38. (canceled)

39. The bicistronic polynucleotide of claim 1, wherein the therapeutic agent comprises an antibody or antigen binding portion thereof, an enzyme, a receptor, a cytokine, a clotting factor, or a hormone.

40. The bicistronic polynucleotide of claim 1, wherein the beta-2-microglobulin (B2M) non-functional polypeptide is a B2M non-functional fragment or a B2M non-functional variant, and/or the human leukocyte antigen (HLA) is HLA-E or HLA-G.

41-45. (canceled)

46. The bicistronic polynucleotide of claim 1, wherein
(i) the nucleic acid sequence encoding the therapeutic agent and the nucleic acid sequence encoding the immune surveillance masking molecule (ISMM) are connect by a 2A element; or,
(ii) the nucleic acid sequence encoding the therapeutic agent and the nucleic acid sequence encoding the immune surveillance masking molecule (ISMM) are connect by an Internal Ribosome Entry Site (IRES); and,
wherein the bicistronic polynucleotide further comprises a 5′ sequence complementary to a B2M gene sequence upstream from an insertion site and a 3′ sequence complementary to a B2M gene sequence downstream from the insertion site, wherein the 5′ sequence and 3′ sequence are at least about 100 nucleotides, at least about 200 nucleotides, at least about 300 nucleotides, at least about 400 nucleotides, at least about 500 nucleotides, at least about 600 nucleotides, at least about 700 nucleotides, at least about 800 nucleotides, at least about 900 nucleotides, at least about 1000 nucleotides in length.

47-49. (canceled)

50. The bicistronic polynucleotide of claim 1, wherein the bicistronic polynucleotide is selected from the group consisting of Bicistronic Construct 1, Bicistronic Construct 2, Bicistronic Construct 3, Bicistronic Construct 4, Bicistronic Construct 5, Bicistronic Construct 6, Bicistronic Construct 7, Bicistronic Construct 8, Full Donor 1, Full Donor 2, Full Donor 3, Full Donor 4, Full Donor 5, Full Donor 6, Full Donor 7, and Full Donor 8.

51-54. (canceled)

55. The bicistronic polynucleotide of claim 54, wherein the bicistronic polynucleotide is inserted in the beta-2-microglobulin (B2M) gene at an exon, intron, or intron-exon junction location, wherein insertion in the B2M gene inactivates the gene.

56-62. (canceled)

63. The bicistronic polynucleotide of claim 55, wherein the insertion site is Site 1 (ACTCTCTCTTTCTGGCCTGG; SEQ ID NO: 1) at exon 1; Site 2 (AGTCACATGGTTCACACGGC; SEQ ID NO:2) or Site 3 (CACAGCCCAAGATAGTTAAG; SEQ ID NO:3) at exon 2; or Site 4 (GAGACATGTAAGCAGCATCA; SEQ ID NO: 4) at exon 3.

64-69. (canceled)

70. A vector comprising a bicistronic polynucleotide of claim 1 operably linked to a regulatory element.

71-75. (canceled)

76. An allogeneic cell genetically modified to express a therapeutic agent and an ISMM, comprising the bicistronic polynucleotide of claim 1, wherein the cell is a T cell, a natural killer (NK) cell, an natural killer T (NKT) cell, an ILC cell, a macrophage, or an antigen presenting cell.

77-81. (canceled)

82. A pharmaceutical composition comprising the cell claim 76.

83-84. (canceled)

85. A method of stimulating a T cell-mediated immune response to a target cell population or tissue in a subject, comprising administering an effective amount of the cell of claim 76 to the subject.

86. A method of providing an anti-tumor immunity in a subject in need thereof, the method comprising administering to the subject an effective amount of the cell of claim 76.

87. A method of treating cancer in a subject in need thereof comprising administering to the subject an effective amount of the cell of claim 76.

88-95. (canceled)

96. A method to generate or expand an allogeneic cell for gene therapy comprising inserting a bicistronic construct comprising a nucleic acid encoding a therapeutic agent and a nucleic acid encoding an immune surveillance masking molecule (ISMM) comprising a nucleic acid encoding a human leukocyte antigen (HLA) selected from HLA-E or HLA-G in the beta-2-microglobulin (B2M) gene, wherein insertion of the bicistronic construct inactivates the B2M gene.

97-99. (canceled)

100. An allogeneic CAR-T cell comprising
(i) a specific bicistronic construct selected from bicistronic construct BC1, BC2, BC3, BC4, BC5, BC6, BC7 or B7, wherein the nucleic acid sequence encoding HLA-E has been replaced with a nucleic acid sequence encoding HLA-G; or,
(ii) a specific bicistronic construct selected from bicistronic construct BC1, BC2, BC3, BC4, BC5, BC6, BC7 or B7, wherein the nucleic acid sequence encoding a fragment of B2M has been replaced with a nucleic acid sequence encoding a fragment of TRAC, and wherein the bicistronic construct is inserted in the TRAC gene; or,
(iii) a specific bicistronic construct selected from bicistronic construct BC1, BC2, BC3, BC4, BC5, BC6, BC7 or B7, wherein the nucleic acid sequence encoding a fragment of B2M has been replaced with a nucleic acid sequence encoding a fragment of CD52, and wherein the bicistronic construct is inserted in the CD52 gene.

101-103. (canceled)


SEQUENCE LISTING (84 sequences)

SEQ ID NO: 1 - DNA, 20 bp, synthetic construct
  source 1..20: mol_type = other DNA
actctctctttctggcctgg

SEQ ID NO: 2 - DNA, 20 bp, synthetic construct
  source 1..20: mol_type = other DNA
agtcacatggttcacacggc

SEQ ID NO: 3 - DNA, 20 bp, synthetic construct
  source 1..20: mol_type = other DNA
cacagcccaagatagttaag

SEQ ID NO: 4 - DNA, 20 bp, synthetic construct
  source 1..20: mol_type = other DNA
gagacatgtaagcagcatca

SEQ ID NO: 5 - DNA, 1584 bp, synthetic construct
  source 1..1584: mol_type = other DNA
  misc_feature 1..1584: note = CAR
atggaactgggactgtcatggatcttcttgctggctatcctgaagggagtgcagtgtgaagttcagctgctgcagagcggacccgaactggaaaaacctggcgcctccgtgatgatcagctgcaaggcctctggcagctccttcaccggctacaacatgaactgggtccgacagaacatcggcaagagcctggaatggatcggcgccatcgatccttactacggcggcaccagctacaaccagaagttcaagggcagagccacactgaccgtggacaagagcagcagcacagcctacatgcatctgaagtccctgaccagcgaggacagcgccgtgtactactgtgtgtccggcatggaatactggggccagggcacaagcgtgacagtctcttctggcggcggtggatctggcggaggcggaagtggtggcggcggatctgatgtggtcatgacacagacccctctgagcctgcctgtgtctctgggagatcaggccagcatcagctgtagaagcagccagagcctggtgcacagaaacggcaacacctacctgcactggtatctgcagaagcccggccagtctcctaagctgctgatccacaaggtgtccaacagattcagcggcgtgcccgacagattctctggctctggaagcggcaccgacttcaccctgaagattagcagagtggaagccgaggacctgggcgtgtacttctgtagccagagcacacacgtgccacctctgacatttggcgctggcaccaagctggaactgaaaaggaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 6 - DNA, 1371 bp, synthetic construct
  source 1..1371: mol_type = other DNA
  misc_feature 1..1371: note = ISMM B2M-HLA-E
atccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggaggcggttctggaggtggcggaagtggcggcgggggaagcggtgggggcggttccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaa

SEQ ID NO: 7 - DNA, 3220 bp, synthetic construct
  source 1..3220: mol_type = other DNA
  misc_feature 1..3220: note = Bicistronic Construct 1
gctagaagctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggaggcggttctggaggtggcggaagtggcggcgggggaagcggtgggggcggttccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgggaagcggagctactaacttcagcctgctgaagcaggctggagacgtggaggagaaccctggacctatggaactgggactgtcatggatcttcttgctggctatcctgaagggagtgcagtgtgaagttcagctgctgcagagcggacccgaactggaaaaacctggcgcctccgtgatgatcagctgcaaggcctctggcagctccttcaccggctacaacatgaactgggtccgacagaacatcggcaagagcctggaatggatcggcgccatcgatccttactacggcggcaccagctacaaccagaagttcaagggcagagccacactgaccgtggacaagagcagcagcacagcctacatgcatctgaagtccctgaccagcgaggacagcgccgtgtactactgtgtgtccggcatggaatactggggccagggcacaagcgtgacagtctcttctggcggcggtggatctggcggaggcggaagtggtggcggcggatctgatgtggtcatgacacagacccctctgagcctgcctgtgtctctgggagatcaggccagcatcagctgtagaagcagccagagcctggtgcacagaaacggcaacacctacctgcactggtatctgcagaagcccggccagtctcctaagctgctgatccacaaggtgtccaacagattcagcggcgtgcccgacagattctctggctctggaagcggcaccgacttcaccctgaagattagcagagtggaagccgaggacctgggcgtgtacttctgtagccagagcacacacgtgccacctctgacatttggcgctggcaccaagctggaactgaaaaggaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctagctgcagggtctggctgttctagaggctgggtttgggaggtgaaacctagtggcaggagaacttgtttattgcagcttataatggttacaaataaagcaatagcatcacaaatttcacaaataaagcatttttttcactgcattctagttgtggtttgtccaaactcatcaatgtatcttatcatgtctg

SEQ ID NO: 8 - DNA, 3670 bp, synthetic construct
  source 1..3670: mol_type = other DNA
  misc_feature 1..3670: note = Bicistronic Construct 2
gctagaagctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggaggcggttctggaggtggcggaagtggcggcgggggaagcggtgggggcggttccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaatccggttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataataggatccatgatggaactgggactgtcatggatcttcttgctggctatcctgaagggagtgcagtgtgaagttcagctgctgcagagcggacccgaactggaaaaacctggcgcctccgtgatgatcagctgcaaggcctctggcagctccttcaccggctacaacatgaactgggtccgacagaacatcggcaagagcctggaatggatcggcgccatcgatccttactacggcggcaccagctacaaccagaagttcaagggcagagccacactgaccgtggacaagagcagcagcacagcctacatgcatctgaagtccctgaccagcgaggacagcgccgtgtactactgtgtgtccggcatggaatactggggccagggcacaagcgtgacagtctcttctggcggcggtggatctggcggaggcggaagtggtggcggcggatctgatgtggtcatgacacagacccctctgagcctgcctgtgtctctgggagatcaggccagcatcagctgtagaagcagccagagcctggtgcacagaaacggcaacacctacctgcactggtatctgcagaagcccggccagtctcctaagctgctgatccacaaggtgtccaacagattcagcggcgtgcccgacagattctctggctctggaagcggcaccgacttcaccctgaagattagcagagtggaagccgaggacctgggcgtgtacttctgtagccagagcacacacgtgccacctctgacatttggcgctggcaccaagctggaactgaaaaggaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctagctgcagggtctggctgttctagaggctgggtttgggaggtgaaacctagtggcaggagaacttgtttattgcagcttataatggttacaaataaagcaatagcatcacaaatttcacaaataaagcatttttttcactgcattctagttgtggtttgtccaaactcatcaatgtatcttatcatgtctg

SEQ ID NO: 9 - DNA, 2969 bp, synthetic construct
  source 1..2969: mol_type = other DNA
  misc_feature 1..2969: note = Bicistronic Construct 3/5
atgccgcgtgaaccatgtgactttgtcacagcccaagatcgtgaaatgggatcgagacatgggtggaggcggttctggaggtggcggaagtggcggcgggggaagcggtgggggcggttccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgggaagcggagctactaacttcagcctgctgaagcaggctggagacgtggaggagaaccctggacctatggaactgggactgtcatggatcttcttgctggctatcctgaagggagtgcagtgtgaagttcagctgctgcagagcggacccgaactggaaaaacctggcgcctccgtgatgatcagctgcaaggcctctggcagctccttcaccggctacaacatgaactgggtccgacagaacatcggcaagagcctggaatggatcggcgccatcgatccttactacggcggcaccagctacaaccagaagttcaagggcagagccacactgaccgtggacaagagcagcagcacagcctacatgcatctgaagtccctgaccagcgaggacagcgccgtgtactactgtgtgtccggcatggaatactggggccagggcacaagcgtgacagtctcttctggcggcggtggatctggcggaggcggaagtggtggcggcggatctgatgtggtcatgacacagacccctctgagcctgcctgtgtctctgggagatcaggccagcatcagctgtagaagcagccagagcctggtgcacagaaacggcaacacctacctgcactggtatctgcagaagcccggccagtctcctaagctgctgatccacaaggtgtccaacagattcagcggcgtgcccgacagattctctggctctggaagcggcaccgacttcaccctgaagattagcagagtggaagccgaggacctgggcgtgtacttctgtagccagagcacacacgtgccacctctgacatttggcgctggcaccaagctggaactgaaaaggaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctagcggtctggctgttctagaggctgggtttgggaggtgaaacctagtggcaggagaacttgtttattgcagcttataatggttacaaataaagcaatagcatcacaaatttcacaaataaagcatttttttcactgcattctagttgtggtttgtccaaactcatcaatgtatcttatcatgtctg

SEQ ID NO: 10 - DNA, 3419 bp, synthetic construct
  source 1..3419: mol_type = other DNA
  misc_feature 1..3419: note = Bicistronic Construct 4/6
atgccgcgtgaaccatgtgactttgtcacagcccaagatcgtgaaatgggatcgagacatgggtggaggcggttctggaggtggcggaagtggcggcgggggaagcggtgggggcggttccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaatccggttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataataggatccatgatggaactgggactgtcatggatcttcttgctggctatcctgaagggagtgcagtgtgaagttcagctgctgcagagcggacccgaactggaaaaacctggcgcctccgtgatgatcagctgcaaggcctctggcagctccttcaccggctacaacatgaactgggtccgacagaacatcggcaagagcctggaatggatcggcgccatcgatccttactacggcggcaccagctacaaccagaagttcaagggcagagccacactgaccgtggacaagagcagcagcacagcctacatgcatctgaagtccctgaccagcgaggacagcgccgtgtactactgtgtgtccggcatggaatactggggccagggcacaagcgtgacagtctcttctggcggcggtggatctggcggaggcggaagtggtggcggcggatctgatgtggtcatgacacagacccctctgagcctgcctgtgtctctgggagatcaggccagcatcagctgtagaagcagccagagcctggtgcacagaaacggcaacacctacctgcactggtatctgcagaagcccggccagtctcctaagctgctgatccacaaggtgtccaacagattcagcggcgtgcccgacagattctctggctctggaagcggcaccgacttcaccctgaagattagcagagtggaagccgaggacctgggcgtgtacttctgtagccagagcacacacgtgccacctctgacatttggcgctggcaccaagctggaactgaaaaggaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctagcggtctggctgttctagaggctgggtttgggaggtgaaacctagtggcaggagaacttgtttattgcagcttataatggttacaaataaagcaatagcatcacaaatttcacaaataaagcatttttttcactgcattctagttgtggtttgtccaaactcatcaatgtatcttatcatgtctg

SEQ ID NO: 11 - DNA, 2924 bp, synthetic construct
  source 1..2924: mol_type = other DNA
  misc_feature 1..2924: note = Bicistronic Construct 7
atcgagacatgggtggaggcggttctggaggtggcggaagtggcggcgggggaagcggtgggggcggttccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgggaagcggagctactaacttcagcctgctgaagcaggctggagacgtggaggagaaccctggacctatggaactgggactgtcatggatcttcttgctggctatcctgaagggagtgcagtgtgaagttcagctgctgcagagcggacccgaactggaaaaacctggcgcctccgtgatgatcagctgcaaggcctctggcagctccttcaccggctacaacatgaactgggtccgacagaacatcggcaagagcctggaatggatcggcgccatcgatccttactacggcggcaccagctacaaccagaagttcaagggcagagccacactgaccgtggacaagagcagcagcacagcctacatgcatctgaagtccctgaccagcgaggacagcgccgtgtactactgtgtgtccggcatggaatactggggccagggcacaagcgtgacagtctcttctggcggcggtggatctggcggaggcggaagtggtggcggcggatctgatgtggtcatgacacagacccctctgagcctgcctgtgtctctgggagatcaggccagcatcagctgtagaagcagccagagcctggtgcacagaaacggcaacacctacctgcactggtatctgcagaagcccggccagtctcctaagctgctgatccacaaggtgtccaacagattcagcggcgtgcccgacagattctctggctctggaagcggcaccgacttcaccctgaagattagcagagtggaagccgaggacctgggcgtgtacttctgtagccagagcacacacgtgccacctctgacatttggcgctggcaccaagctggaactgaaaaggaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctagctgcagggtctggctgttctagaggctgggtttgggaggtgaaacctagtggcaggagaacttgtttattgcagcttataatggttacaaataaagcaatagcatcacaaatttcacaaataaagcatttttttcactgcattctagttgtggtttgtccaaactcatcaatgtatcttatcatgtctg

SEQ ID NO: 12 - DNA, 3374 bp, synthetic construct
  source 1..3374: mol_type = other DNA
  misc_feature 1..3374: note = Bicistronic Construct 8
atcgagacatgggtggaggcggttctggaggtggcggaagtggcggcgggggaagcggtgggggcggttccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaatccggttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataataggatccatgatggaactgggactgtcatggatcttcttgctggctatcctgaagggagtgcagtgtgaagttcagctgctgcagagcggacccgaactggaaaaacctggcgcctccgtgatgatcagctgcaaggcctctggcagctccttcaccggctacaacatgaactgggtccgacagaacatcggcaagagcctggaatggatcggcgccatcgatccttactacggcggcaccagctacaaccagaagttcaagggcagagccacactgaccgtggacaagagcagcagcacagcctacatgcatctgaagtccctgaccagcgaggacagcgccgtgtactactgtgtgtccggcatggaatactggggccagggcacaagcgtgacagtctcttctggcggcggtggatctggcggaggcggaagtggtggcggcggatctgatgtggtcatgacacagacccctctgagcctgcctgtgtctctgggagatcaggccagcatcagctgtagaagcagccagagcctggtgcacagaaacggcaacacctacctgcactggtatctgcagaagcccggccagtctcctaagctgctgatccacaaggtgtccaacagattcagcggcgtgcccgacagattctctggctctggaagcggcaccgacttcaccctgaagattagcagagtggaagccgaggacctgggcgtgtacttctgtagccagagcacacacgtgccacctctgacatttggcgctggcaccaagctggaactgaaaaggaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctagctgcagggtctggctgttctagaggctgggtttgggaggtgaaacctagtggcaggagaacttgtttattgcagcttataatggttacaaataaagcaatagcatcacaaatttcacaaataaagcatttttttcactgcattctagttgtggtttgtccaaactcatcaatgtatcttatcatgtctg

SEQ ID NO: 13 - DNA, 4202 bp, synthetic construct
  source 1..4202: mol_type = other DNA
  misc_feature 1..4202: note = Full Donor Construct 1
agatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagatgtctcgctccgtggccttagctgtgctcgcgctactctctctttctgggctagaagctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggaggcggttctggaggtggcggaagtggcggcgggggaagcggtgggggcggttccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgggaagcggagctactaacttcagcctgctgaagcaggctggagacgtggaggagaaccctggacctatggaactgggactgtcatggatcttcttgctggctatcctgaagggagtgcagtgtgaagttcagctgctgcagagcggacccgaactggaaaaacctggcgcctccgtgatgatcagctgcaaggcctctggcagctccttcaccggctacaacatgaactgggtccgacagaacatcggcaagagcctggaatggatcggcgccatcgatccttactacggcggcaccagctacaaccagaagttcaagggcagagccacactgaccgtggacaagagcagcagcacagcctacatgcatctgaagtccctgaccagcgaggacagcgccgtgtactactgtgtgtccggcatggaatactggggccagggcacaagcgtgacagtctcttctggcggcggtggatctggcggaggcggaagtggtggcggcggatctgatgtggtcatgacacagacccctctgagcctgcctgtgtctctgggagatcaggccagcatcagctgtagaagcagccagagcctggtgcacagaaacggcaacacctacctgcactggtatctgcagaagcccggccagtctcctaagctgctgatccacaaggtgtccaacagattcagcggcgtgcccgacagattctctggctctggaagcggcaccgacttcaccctgaagattagcagagtggaagccgaggacctgggcgtgtacttctgtagccagagcacacacgtgccacctctgacatttggcgctggcaccaagctggaactgaaaaggaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctagctgcagggtctggctgttctagaggctgggtttgggaggtgaaacctagtggcaggagaacttgtttattgcagcttataatggttacaaataaagcaatagcatcacaaatttcacaaataaagcatttttttcactgcattctagttgtggtttgtccaaactcatcaatgtatcttatcatgtctggaggctatccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgct

SEQ ID NO: 14 - DNA, 4652 bp, synthetic construct
  source 1..4652: mol_type = other DNA
  misc_feature 1..4652: note = Full Donor Construct 2
agatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagatgtctcgctccgtggccttagctgtgctcgcgctactctctctttctgggctagaagctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggaggcggttctggaggtggcggaagtggcggcgggggaagcggtgggggcggttccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaatccggttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataataggatccatgatggaactgggactgtcatggatcttcttgctggctatcctgaagggagtgcagtgtgaagttcagctgctgcagagcggacccgaactggaaaaacctggcgcctccgtgatgatcagctgcaaggcctctggcagctccttcaccggctacaacatgaactgggtccgacagaacatcggcaagagcctggaatggatcggcgccatcgatccttactacggcggcaccagctacaaccagaagttcaagggcagagccacactgaccgtggacaagagcagcagcacagcctacatgcatctgaagtccctgaccagcgaggacagcgccgtgtactactgtgtgtccggcatggaatactggggccagggcacaagcgtgacagtctcttctggcggcggtggatctggcggaggcggaagtggtggcggcggatctgatgtggtcatgacacagacccctctgagcctgcctgtgtctctgggagatcaggccagcatcagctgtagaagcagccagagcctggtgcacagaaacggcaacacctacctgcactggtatctgcagaagcccggccagtctcctaagctgctgatccacaaggtgtccaacagattcagcggcgtgcccgacagattctctggctctggaagcggcaccgacttcaccctgaagattagcagagtggaagccgaggacctgggcgtgtacttctgtagccagagcacacacgtgccacctctgacatttggcgctggcaccaagctggaactgaaaaggaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctagctgcagggtctggctgttctagaggctgggtttgggaggtgaaacctagtggcaggagaacttgtttattgcagcttataatggttacaaataaagcaatagcatcacaaatttcacaaataaagcatttttttcactgcattctagttgtggtttgtccaaactcatcaatgtatcttatcatgtctggaggctatccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgct

SEQ ID NO: 15 - DNA, 3949 bp, synthetic construct
  source 1..3949: mol_type = other DNA
  misc_feature 1..3949: note = Full Donor Construct 3
tctgctagaaaaaaaacaaaaaaggcatgtatagaggaattatgagggaaagataccaagtcacggtttattcttcaaaatggaggtggcttgttgggaaggtggaagctcatttggccagagtggaaatggaattgggagaaatcgatgaccaaatgtaaacacttggtgcctgatatagcttgacaccaagttagccccaagtgaaataccctggcaatattaatgtgtcttttcccgatattcctcaggtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcatgccgcgtgaaccatgtgactttgtcacagcccaagatcgtgaaatgggatcgagacatgggtggaggcggttctggaggtggcggaagtggcggcgggggaagcggtgggggcggttccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgggaagcggagctactaacttcagcctgctgaagcaggctggagacgtggaggagaaccctggacctatggaactgggactgtcatggatcttcttgctggctatcctgaagggagtgcagtgtgaagttcagctgctgcagagcggacccgaactggaaaaacctggcgcctccgtgatgatcagctgcaaggcctctggcagctccttcaccggctacaacatgaactgggtccgacagaacatcggcaagagcctggaatggatcggcgccatcgatccttactacggcggcaccagctacaaccagaagttcaagggcagagccacactgaccgtggacaagagcagcagcacagcctacatgcatctgaagtccctgaccagcgaggacagcgccgtgtactactgtgtgtccggcatggaatactggggccagggcacaagcgtgacagtctcttctggcggcggtggatctggcggaggcggaagtggtggcggcggatctgatgtggtcatgacacagacccctctgagcctgcctgtgtctctgggagatcaggccagcatcagctgtagaagcagccagagcctggtgcacagaaacggcaacacctacctgcactggtatctgcagaagcccggccagtctcctaagctgctgatccacaaggtgtccaacagattcagcggcgtgcccgacagattctctggctctggaagcggcaccgacttcaccctgaagattagcagagtggaagccgaggacctgggcgtgtacttctgtagccagagcacacacgtgccacctctgacatttggcgctggcaccaagctggaactgaaaaggaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctagcggtctggctgttctagaggctgggtttgggaggtgaaacctagtggcaggagaacttgtttattgcagcttataatggttacaaataaagcaatagcatcacaaatttcacaaataaagcatttttttcactgcattctagttgtggtttgtccaaactcatcaatgtatcttatcatgtctggtaagtcttacattcttttgtaagctgctgaaagttgtgtatgagtagtcatatcataaagctgctttgatataaaaaaggtctatggccatactaccctgaatgagtcccatcccatctgatataaacaatctgcatattgggattgtcagggaatgttcttaaagatcagattagtggcacctgctgagatactgatgcacagcatggtttctgaaccagtagtttccctgcagttgagcagggagcagcagcagcacttgcacaaatacatatacactcttaacacttcttacctactggcttcctctagcttttgtggcagcttcaggtatatttagcactgaacgaacatctcaagaaggtataggcctttgtttgtaagtcctgctgtcctagcatcctataatcctggacttctccagtactttctggctggattggtatctgaggctagtaggaagggcttgttcctgctgggtagctctaaacaatgtatt

SEQ ID NO: 16 - DNA, 4399 bp, synthetic construct
  source 1..4399: mol_type = other DNA
  misc_feature 1..4399: note = Full Donor Construct 6
tctgctagaaaaaaaacaaaaaaggcatgtatagaggaattatgagggaaagataccaagtcacggtttattcttcaaaatggaggtggcttgttgggaaggtggaagctcatttggccagagtggaaatggaattgggagaaatcgatgaccaaatgtaaacacttggtgcctgatatagcttgacaccaagttagccccaagtgaaataccctggcaatattaatgtgtcttttcccgatattcctcaggtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcatgccgcgtgaaccatgtgactttgtcacagcccaagatcgtgaaatgggatcgagacatgggtggaggcggttctggaggtggcggaagtggcggcgggggaagcggtgggggcggttccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaatccggttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataataggatccatgatggaactgggactgtcatggatcttcttgctggctatcctgaagggagtgcagtgtgaagttcagctgctgcagagcggacccgaactggaaaaacctggcgcctccgtgatgatcagctgcaaggcctctggcagctccttcaccggctacaacatgaactgggtccgacagaacatcggcaagagcctggaatggatcggcgccatcgatccttactacggcggcaccagctacaaccagaagttcaagggcagagccacactgaccgtggacaagagcagcagcacagcctacatgcatctgaagtccctgaccagcgaggacagcgccgtgtactactgtgtgtccggcatggaatactggggccagggcacaagcgtgacagtctcttctggcggcggtggatctggcggaggcggaagtggtggcggcggatctgatgtggtcatgacacagacccctctgagcctgcctgtgtctctgggagatcaggccagcatcagctgtagaagcagccagagcctggtgcacagaaacggcaacacctacctgcactggtatctgcagaagcccggccagtctcctaagctgctgatccacaaggtgtccaacagattcagcggcgtgcccgacagattctctggctctggaagcggcaccgacttcaccctgaagattagcagagtggaagccgaggacctgggcgtgtacttctgtagccagagcacacacgtgccacctctgacatttggcgctggcaccaagctggaactgaaaaggaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctagcggtctggctgttctagaggctgggtttgggaggtgaaacctagtggcaggagaacttgtttattgcagcttataatggttacaaataaagcaatagcatcacaaatttcacaaataaagcatttttttcactgcattctagttgtggtttgtccaaactcatcaatgtatcttatcatgtctggtaagtcttacattcttttgtaagctgctgaaagttgtgtatgagtagtcatatcataaagctgctttgatataaaaaaggtctatggccatactaccctgaatgagtcccatcccatctgatataaacaatctgcatattgggattgtcagggaatgttcttaaagatcagattagtggcacctgctgagatactgatgcacagcatggtttctgaaccagtagtttccctgcagttgagcagggagcagcagcagcacttgcacaaatacatatacactcttaacacttcttacctactggcttcctctagcttttgtggcagcttcaggtatatttagcactgaacgaacatctcaagaaggtataggcctttgtttgtaagtcctgctgtcctagcatcctataatcctggacttctccagtactttctggctggattggtatctgaggctagtaggaagggcttgttcctgctgggtagctctaaacaatgtatt

SEQ ID NO: 17 - DNA, 3920 bp, synthetic construct
  source 1..3920: mol_type = other DNA
  misc_feature 1..3920: note = Full Donor Construct 7
tctgcatattgggattgtcagggaatgttcttaaagatcagattagtggcacctgctgagatactgatgcacagcatggtttctgaaccagtagtttccctgcagttgagcagggagcagcagcagcacttgcacaaatacatatacactcttaacacttcttacctactggcttcctctagcttttgtggcagcttcaggtatatttagcactgaacgaacatctcaagaaggtataggcctttgtttgtaagtcctgctgtcctagcatcctataatcctggacttctccagtactttctggctggattggtatctgaggctagtaggaagggcttgttcctgctgggtagctctaaacaatgtattcatgggtaggaacagcagcctattctgccagccttatttctaaccattttagacatttgttagtacatggtattttaaaagtaaaacttaatgtcttccttttttttctccactgtctttttcatagatcgagacatgggtggaggcggttctggaggtggcggaagtggcggcgggggaagcggtgggggcggttccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgggaagcggagctactaacttcagcctgctgaagcaggctggagacgtggaggagaaccctggacctatggaactgggactgtcatggatcttcttgctggctatcctgaagggagtgcagtgtgaagttcagctgctgcagagcggacccgaactggaaaaacctggcgcctccgtgatgatcagctgcaaggcctctggcagctccttcaccggctacaacatgaactgggtccgacagaacatcggcaagagcctggaatggatcggcgccatcgatccttactacggcggcaccagctacaaccagaagttcaagggcagagccacactgaccgtggacaagagcagcagcacagcctacatgcatctgaagtccctgaccagcgaggacagcgccgtgtactactgtgtgtccggcatggaatactggggccagggcacaagcgtgacagtctcttctggcggcggtggatctggcggaggcggaagtggtggcggcggatctgatgtggtcatgacacagacccctctgagcctgcctgtgtctctgggagatcaggccagcatcagctgtagaagcagccagagcctggtgcacagaaacggcaacacctacctgcactggtatctgcagaagcccggccagtctcctaagctgctgatccacaaggtgtccaacagattcagcggcgtgcccgacagattctctggctctggaagcggcaccgacttcaccctgaagattagcagagtggaagccgaggacctgggcgtgtacttctgtagccagagcacacacgtgccacctctgacatttggcgctggcaccaagctggaactgaaaaggaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctagctgcagggtctggctgttctagaggctgggtttgggaggtgaaacctagtggcaggagaacttgtttattgcagcttataatggttacaaataaagcaatagcatcacaaatttcacaaataaagcatttttttcactgcattctagttgtggtttgtccaaactcatcaatgtatcttatcatgtctgaggtaagtttttgaccttgagaaaatgtttttgtttcactgtcctgaggactatttatagacagctctaacatgataaccctcactatgtggagaacattgacagagtaacattttagcagggaaagaagaatcctacagggtcatgttcccttctcctgtggagtggcatgaagaaggtgtatggccccaggtatggccatattactgaccctctacagagagggcaaaggaactgccagtatggtattgcaggataaaggcaggtggttacccacattacctgcaaggctttgatctttcttctgccatttccacattggacatctctgctgaggagagaaaatgaaccactcttttcctttgtataatgttgttttattcttcagacagaagagaggagttatacagctctgcagacatcccattcctgtatggggactgtgtttgcctcttagaggttcccaggccactagaggagataaagggaaacagattgtt

SEQ ID NO: 18 - DNA, 3920 bp, synthetic construct; identical to SEQ ID NO: 17
  source 1..3920: mol_type = other DNA
  misc_feature 1..3920: note = Full Donor Construct 8

SEQ ID NO: 19 - DNA, 1584 bp, synthetic construct; identical to SEQ ID NO: 5
  source 1..1584: mol_type = other DNA
  misc_feature 1..1584: note = anti-GD2 CAR

SEQ ID NO: 20 - protein, 528 aa, synthetic construct
  REGION 1..528: note = anti-GD2 CAR
MELGLSWIFLLAILKGVQCEVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSGGGGSGGGGSGGGGSDVVMTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - DNA, 57 bp, synthetic construct
  source 1..57: mol_type = other DNA
  misc_feature 1..57: note = Signal peptide
atggaactgggactgtcatggatcttcttgctggctatcctgaagggagtgcagtgt

SEQ ID NO: 22 - protein, 19 aa, synthetic construct
  REGION 1..19: note = Signal peptide
MELGLSWIFLLAILKGVQC

SEQ ID NO: 23 - DNA, 726 bp, synthetic construct
  source 1..726: mol_type = other DNA
  misc_feature 1..726: note = anti- GD2scFv
gaagttcagctgctgcagagcggacccgaactggaaaaacctggcgcctccgtgatgatcagctgcaaggcctctggcagctccttcaccggctacaacatgaactgggtccgacagaacatcggcaagagcctggaatggatcggcgccatcgatccttactacggcggcaccagctacaaccagaagttcaagggcagagccacactgaccgtggacaagagcagcagcacagcctacatgcatctgaagtccctgaccagcgaggacagcgccgtgtactactgtgtgtccggcatggaatactggggccagggcacaagcgtgacagtctcttctggcggcggtggatctggcggaggcggaagtggtggcggcggatctgatgtggtcatgacacagacccctctgagcctgcctgtgtctctgggagatcaggccagcatcagctgtagaagcagccagagcctggtgcacagaaacggcaacacctacctgcactggtatctgcagaagcccggccagtctcctaagctgctgatccacaaggtgtccaacagattcagcggcgtgcccgacagattctctggctctggaagcggcaccgacttcaccctgaagattagcagagtggaagccgaggacctgggcgtgtacttctgtagccagagcacacacgtgccacctctgacatttggcgctggcaccaagctggaactgaaaagg

SEQ ID NO: 24 - protein, 242 aa, synthetic construct
  REGION 1..242: note = anti- GD2scFv
EVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSGGGGSGGGGSGGGGSDVVMTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKR

SEQ ID NO: 25 - DNA, 135 bp, synthetic construct
  source 1..135: mol_type = other DNA
  misc_feature 1..135: note = CD8alpha hinge
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 26 - protein, 45 aa, synthetic construct
  REGION 1..45: note = CD8alpha hinge
TTTPAPRPPTPAPTIASQPLSLRPEACRPAAGGAVHTRGLDFACD

SEQ ID NO: 27 - DNA, 204 bp, synthetic construct
  source 1..204: mol_type = other DNA
  misc_feature 1..204: note = CD28 TM/IC domains
ttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 28 - protein, 68 aa, synthetic construct
  REGION 1..68: note = CD28 TM/IC domains
FWVLVVVGGVLACYSLLVTVAFIIFWVRSKRSRLLHSDYMNMTPRRPGPTRKHYQPYAPPRDFAAYRS

SEQ ID NO: 29 - DNA, 126 bp, synthetic construct
  source 1..126: mol_type = other DNA
  misc_feature 1..126: note = 4-IBB activation domain
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 30 - protein, 42 aa, synthetic construct
  REGION 1..42: note = 4-IBB activation domain
KRGRKKLLYIFKQPFMRPVQTTQEEDGCSCRFPEEEEGGCEL

SEQ ID NO: 31 - DNA, 336 bp, synthetic construct
  source 1..336: mol_type = other DNA
  misc_feature 1..336: note = CD3zeta activation domain
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 32 - protein, 112 aa, synthetic construct
  REGION 1..112: note = CD3zeta activation domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - DNA, 1371 bp, synthetic construct; identical to SEQ ID NO: 6
  source 1..1371: mol_type = other DNA
  misc_feature 1..1371: note = HLA-E dimer

SEQ ID NO: 34 - protein, 456 aa, synthetic construct
  REGION 1..456: note = HLA-E dimer
IQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 35 - DNA, 357 bp, synthetic construct
  source 1..357: mol_type = other DNA
  misc_feature 1..357: note = Beta2mi croglobulin
atgtctcgctccgtggccttagctgtgctcgcgctactctctctttctgggctagaagctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatg

SEQ ID NO: 36 - protein, 119 aa, synthetic construct
  REGION 1..119: note = Beta2mi croglobulin
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 37 - DNA, 60 bp, synthetic construct
  source 1..60: mol_type = other DNA
  misc_feature 1..60: note = 4X G4S linker
ggtggaggcggttctggaggtggcggaagtggcggcgggggaagcggtgggggcggttcc

SEQ ID NO: 38 - protein, 20 aa, synthetic construct
  REGION 1..20: note = 4X G4S linker
GGGGSGGGGSGGGGSGGGGS

SEQ ID NO: 39 - DNA, 1014 bp, synthetic construct
  source 1..1014: mol_type = other DNA
  misc_feature 1..1014: note = Mature HLA-E
ggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaa

SEQ ID NO: 40 - protein, 337 aa, synthetic construct
  REGION 1..337: note = Mature HLA-E
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 41 - protein, 443 aa, synthetic construct
  REGION 1..443: note = Dinutuximab Chimeric lgG 1-Kappa Heavy Chain
EVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 220 aa, synthetic construct
  REGION 1..220: note = Dinutuximab Chimeric lgG 1-Kappa Light Chain
EIVMTQSPATLSVSPGERATLSCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - DNA, 339 bp, synthetic construct
  source 1..339: mol_type = other DNA
  misc_feature 1..339: note = VH
gaagttcagctgctgcagagcggacccgaactggaaaaacctggcgcctccgtgatgatcagctgcaaggcctctggcagctccttcaccggctacaacatgaactgggtccgacagaacatcggcaagagcctggaatggatcggcgccatcgatccttactacggcggcaccagctacaaccagaagttcaagggcagagccacactgaccgtggacaagagcagcagcacagcctacatgcatctgaagtccctgaccagcgaggacagcgccgtgtactactgtgtgtccggcatggaatactggggccagggcacaagcgtgacagtctcttct

SEQ ID NO: 44 - protein, 113 aa, synthetic construct [sequence omitted]

SEQ ID NO: 45 - protein, 339 aa, synthetic construct
  REGION 1..339: note = VL
GAAATCGTAATGACTCAGAGCCCGGCTACCCTTTCCGTCTCACCGGGAGAGAGAGCAACCCTTAGCTGTAGAAGCAGCCAGAGCCTGGTGCACAGAAACGGCAACACCTACCTGCACTGGTATCTGCAGAAGCCCGGCCAGTCTCCTAAGCTGCTGATCCACAAGGTGTCCAACAGATTCAGCGGCGTGCCCGACAGATTCTCTGGCTCTGGAAGCGGCACCGACTTCACCCTGAAGATTAGCAGAGTGGAAGCCGAGGACCTGGGCGTGTACTTCTGTAGCCAGAGCACACACGTGCCACCTCTGACATTTGGCGCTGGCACCAAGCTGGAACTGAAA

SEQ ID NO: 46 - protein, 113 aa, synthetic construct [sequence omitted]

SEQ ID NO: 47 - protein, 8 aa, synthetic construct
  REGION 1..8: note = VHCDRl
GSSFTGYN

SEQ ID NO: 48 - protein, 8 aa, synthetic construct
  REGION 1..8: note = VHCDR2
IDPYYGGT

SEQ ID NO: 49 - protein, 6 aa, synthetic construct
  REGION 1..6: note = VHCDR3
VSGMEY

SEQ ID NO: 50 - protein, 11 aa, synthetic construct
  REGION 1..11: note = VL CDR1
QSLVHRNGNTY

SEQ ID NO: 51 - skipped

SEQ ID NO: 52 - protein, 10 aa, synthetic construct
  REGION 1..10: note = VL CDR3
SQSTHVPPLT

SEQ ID NO: 53 - protein, 8 aa, synthetic construct
  REGION 1..8: note = VH CDR1 Contact
GSSFTGYN

SEQ ID NO: 54 - protein, 8 aa, synthetic construct
  REGION 1..8: note = VH CDR2 Contact
IDPYYGGT

SEQ ID NO: 55 - protein, 6 aa, synthetic construct
  REGION 1..6: note = VH CDR3 IMGT
VSGMEY

SEQ ID NO: 56 - protein, 11 aa, synthetic construct
  REGION 1..11: note = VL CDR1 IMGT; note = VL CDR1
QSLVHRNGNTY

SEQ ID NO: 57 - skipped

SEQ ID NO: 58 - protein, 10 aa, synthetic construct
  REGION 1..10: note = VL CDR3 IMGT
SQSTHVPPLT

SEQ ID NO: 59 - protein, 7 aa, synthetic construct
  REGION 1..7: note = VH CDR1 Chothia
GSSFTGY

SEQ ID NO: 60 - protein, 10 aa, synthetic construct
  REGION 1..10: note = VH CDR1 AbM
GSSFTGYNMN

SEQ ID NO: 61 - protein, 5 aa, synthetic construct
  REGION 1..5: note = VH CDR1 Kabat
GYNMN

SEQ ID NO: 62 - protein, 6 aa, synthetic construct
  REGION 1..6: note = VH CDR1 Contact
TGYNMN

SEQ ID NO: 63 - protein, 6 aa, synthetic construct
  REGION 1..6: note = VH CDR2 Chothia
DPYYGG

SEQ ID NO: 64 - protein, 10 aa, synthetic construct
  REGION 1..10: note = VH CDR2 AbM
AIDPYYGGTS

SEQ ID NO: 65 - protein, 17 aa, synthetic construct
  CONFLICT 1..17: note = VH CDR2 Kabat
AIDPYYGGTSYNQKFKG

SEQ ID NO: 66 - protein, 13 aa, synthetic construct
  REGION 1..13: note = VH CDR2 Contact
WIGAIDPYYGGTS

SEQ ID NO: 67 - protein, 4 aa, synthetic construct
  REGION 1..4: note = VH CDR3 Chothia
GMEY

SEQ ID NO: 68 - protein, 4 aa, synthetic construct
  REGION 1..4: note = VH CDR3 AbM
GMEY

SEQ ID NO: 69 - protein, 4 aa, synthetic construct
  REGION 1..4: note = VH CDR3 Kabat
GMEY

SEQ ID NO: 70 - protein, 5 aa, synthetic construct
  REGION 1..5: note = VH CDR3 Contact
VSGME

SEQ ID NO: 71 - protein, 16 aa, synthetic construct
  REGION 1..16: note = VL CDR1 Chothia
RSSQSLVHRNGNTYLH

SEQ ID NO: 72 - protein, 16 aa, synthetic construct
  REGION 1..16: note = VL CDR1 AbM
RSSQSLVHRNGNTYLH

SEQ ID NO: 73 - protein, 16 aa, synthetic construct
  REGION 1..16: note = VL CDR1 Kabat
RSSQSLVHRNGNTYLH

SEQ ID NO: 74 - protein, 12 aa, synthetic construct
  REGION 1..12: note = VL CDR1 Contact
VHRNGNTYLHWY

SEQ ID NO: 75 - protein, 7 aa, synthetic construct
  REGION 1..7: note = VL CDR2 Chothia
KVSNRFS

SEQ ID NO: 76 - protein, 7 aa, synthetic construct
  REGION 1..7: note = VL CDR2 AbM
KVSNRFS

SEQ ID NO: 77 - protein, 7 aa, synthetic construct
  REGION 1..7: note = VL CDR2 Kabat
KVSNRFS

SEQ ID NO: 78 - protein, 10 aa, synthetic construct
  REGION 1..10: note = VL CDR2 Contact
LLIHKVSNRF

SEQ ID NO: 79 - protein, 10 aa, synthetic construct
  REGION 1..10: note = VL CDR3 Chothia
SQSTHVPPLT

SEQ ID NO: 80 - protein, 10 aa, synthetic construct
  REGION 1..10: note = VL CDR3 AbM
SQSTHVPPLT

SEQ ID NO: 81 - protein, 10 aa, synthetic construct
  REGION 1..10: note = VL CDR3 Kabat
SQSTHVPPLT

SEQ ID NO: 82 - protein, 9 aa, synthetic construct
  REGION 1..9: note = VL CDR3 Contact
SQSTHVPPL

SEQ ID NO: 83 - protein, 4 aa, synthetic construct
  REGION 1..4: note = Gly4-Ser linker
GSSS

SEQ ID NO: 84 - protein, 16 aa, synthetic construct
  REGION 1..16: note = Gly4-Ser linker
GSSSGSSSGSSSGSSS